(19) United States
(12) Patent Application Publication
(10) Pub. No.: US 2024/0052430 A1
(43) Pub. Date: Feb. 15, 2024

(54) THERAPEUTIC AND DIAGNOSTIC METHODS AND COMPOSITIONS FOR CANCER

(71) Applicants: Genentech, Inc., South San Francisco, CA (US); Icahn School of Medicine at Mount Sinai, New York, NY (US)

(72) Inventors: Christian HAMMER, South San Francisco, CA (US); Amir HOROWITZ, New York, NY (US)

(21) Appl. No.: 18/496,065

(22) Filed: Oct. 27, 2023

Related U.S. Application Data
(63) Continuation of application No. PCT/US2022/026919, filed on Apr. 29, 2022.
(60) Provisional application No. 63/256,873, filed on Oct. 18, 2021.
(60) Provisional application No. 63/226,634, filed on Jul. 28, 2021.
(60) Provisional application No. 63/182,307, filed on Apr. 30, 2021.

(51) Int. Cl. C12Q 1/6886; C07K 16/28; A61P 35/00; A61K 31/337; A61K 31/282; C07K 16/22; A61K 39/395

(52) U.S. Cl. CPC C12Q 1/6886; C07K 16/2827; C07K 16/2818; A61P 35/00; A61K 31/337; A61K 31/282; C07K 16/22; A61K 39/3955; C12Q 2600/106; C12Q 2600/156; C12Q 2600/158; C07K 2317/56; C07K 2317/24; C07K 2317/21; A61K 2039/545

(57) ABSTRACT

The invention provides methods and compositions for treating cancer (e.g., NSCLC) in a patient, for example, by administering a treatment regimen that includes a PD-1 axis binding antagonist (e.g., atezolizumab) or an NK cell-directed therapy agent to the patient. Also provided are compositions (e.g., a PD-1 axis binding antagonist (e.g., atezolizumab) or an NK cell-directed therapy agent, pharmaceutical compositions thereof, kits thereof, and articles of manufacture thereof) for use in treating cancer (e.g., NSCLC) in a patient. Also provided are methods for identifying a cancer (e.g., NSCLC) patient who may benefit from treatment with a treatment regimen that includes a PD-1 axis binding antagonist (e.g., atezolizumab) or an NK cell-directed therapy agent. Also provided are methods for selecting a therapy for a cancer patient.

### CROSS-REFERENCE TO RELATED APPLICATIONS

This application is a continuation of International Patent Application No. PCT/US2022/026919, filed on Apr. 29, 2022, which claims priority to U.S. Patent Application No. 63/256,873, filed on Oct. 18, 2021, U.S. Patent Application No. 63/226,634, filed on Jul. 28, 2021, and U.S. Patent Application No. 63/182,307, filed on Apr. 30, 2021, the disclosures of which are incorporated herein by reference in their entirety.

### SEQUENCE LISTING

The instant application contains a Sequence Listing which has been submitted electronically in XML format and is hereby incorporated by reference in its entirety. Said XML copy, created on Oct. 19, 2023, is named 50474-256005_Sequence_Listing_1019_23 and is 10,407 bytes in size.

### FIELD OF THE INVENTION

This invention relates to methods and compositions for use in treating and diagnosing cancer (e.g., non-small cell lung cancer (NSCLC) in a patient, for example, by administering to the patient a treatment regimen that includes a PD-1 axis binding antagonist (e.g., atezolizumab) and/or an NK cell-directed therapy agent, alone or in combination with a taxane (e.g., nab-paclitaxel or paclitaxel), a platinum-based chemotherapeutic agent (e.g., carboplatin), and/or an anti-angiogenic agent (e.g., bevacizumab).

### BACKGROUND OF THE INVENTION

Cancer remains one of the deadliest threats to human health. In the U.S., cancer affects nearly 1.3 million new patients each year and is the second leading cause of death after heart disease, accounting for approximately 1 in 4 deaths. Solid tumors are responsible for most of those deaths. For example, lung cancer is the leading cause of cancer deaths worldwide. It was estimated that there were 224,210 new cases of lung cancer (116,000 in men and 108,210 in women) and 159,260 deaths in the United States in 2014. Similar data from Europe estimate that there were 214,000 new cases of lung cancer and 268,000 deaths in 2012. NSCLC is one of the two major types of lung cancer, accounting for approximately 85% of all lung cancer cases. The two predominant histologic types of NSCLC are adenocarcinoma, which accounts for more than half of cases, and squamous cell carcinoma, which accounts for approximately 25% of cases.

Accordingly, there is a need in the art for improved therapies for cancer (e.g., NSCLC).

### SUMMARY OF THE INVENTION

The invention provides, inter alia, methods, compositions for use, uses, and articles of manufacture for treating and diagnosing cancer.

In one aspect, the invention features a method of treating non-small cell lung cancer (NSCLC) in a patient in need thereof whose genome has been determined to comprise at least one copy of HLA-C1, the method comprising administering to the patient an effective amount of a treatment regimen comprising a PD-1 axis binding antagonist.

In another aspect, the invention features a PD-1 axis binding antagonist for use in treating NSCLC in a patient in need thereof whose genome has been determined to comprise at least one copy of HLA-C1.

In some aspects, the patient's genome further comprises at least one copy of KIR2DL3.

In another aspect, the invention features a method of treating NSCLC in a patient in need thereof whose genome has been determined to comprise at least one copy of HLA-C1 and at least one copy of KIR2DL3, the method comprising administering to the patient an effective amount of a treatment regimen comprising a PD-1 axis binding antagonist.

In another aspect, the invention features a PD-1 axis binding antagonist for use in treating NSCLC in a patient in need thereof whose genome has been determined to comprise at least one copy of HLA-C1 and at least one copy of KIR2DL3.

In another aspect, the invention features a method of treating NSCLC in a patient in need thereof whose genome has been determined to comprise at least one copy of HLA-Bw4, the method comprising administering to the patient an effective amount of a treatment regimen comprising a PD-1 axis binding antagonist.

In another aspect, the invention features a PD-1 axis binding antagonist for use in treating NSCLC in a patient in need thereof whose genome has been determined to comprise at least one copy of HLA-Bw4.

In some aspects, the patient's genome further comprises at least one copy of KIR3DL1.

In another aspect, the invention features a method of treating NSCLC in a patient in need thereof whose genome has been determined to comprise at least one copy of HLA-Bw4 and at least one copy of KIR3DL1, the method comprising administering to the patient an effective amount of a treatment regimen comprising a PD-1 axis binding antagonist.

In another aspect, the invention features a PD-1 axis binding antagonist for use in treating NSCLC in a patient in need thereof whose genome has been determined to comprise at least one copy of HLA-Bw4 and at least one copy of KIR3DL1.

In another aspect, the invention features a method of treating NSCLC in a patient in need thereof, the method comprising: (a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-C1 in the patient's genome.

In another aspect, the invention features a PD-1 axis binding antagonist for use in a method of treating NSCLC in a patient in need thereof, the method comprising: (a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-C1 in the patient's genome.

In some aspects, step (a) further comprises determining whether the patient's genome comprises at least one copy of KIR2DL3.

In another aspect, the invention features a method of treating NSCLC in a patient in need thereof, the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-C1 and at least one copy of KIR2DL3 in the patient's genome.

In another aspect, the invention features a PD-1 axis binding antagonist for use in treating NSCLC in a patient in need thereof, the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-C1 and at least one copy of KIR2DL3 in the patient's genome.

In another aspect, the invention features a method of treating NSCLC in a patient in need thereof, the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-Bw4 in the patient's genome.

In another aspect, the invention features a PD-1 axis binding antagonist for use in treating NSCLC in a patient in need thereof, the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-Bw4 in the patient's genome.

In some aspects, step (a) further comprises determining whether the patient's genome comprises at least one copy of KIR3DL1.

In another aspect, the invention features a method of treating NSCLC in a patient in need thereof, the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-Bw4 and at least one copy of KIR3DL1 in the patient's genome.

In another aspect, the invention features a PD-1 axis binding antagonist for use in treating NSCLC in a patient in need thereof, the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-Bw4 and at least one copy of KIR3DL1 in the patient's genome.

In another aspect, the invention features a method of identifying a patient having NSCLC who may benefit from a treatment regimen comprising a PD-1 axis binding antagonist, the method comprising determining whether the patient's genome comprises at least one copy of HLA-C1, wherein the presence of at least one copy of HLA-C1 in the patient's genome identifies the patient as one who may benefit from treatment with a treatment regimen comprising a PD-1 axis binding antagonist.

In another aspect, the invention features a method of identifying a patient having NSCLC who may benefit from a treatment regimen comprising a PD-1 axis binding antagonist, the method comprising: (a) performing germline whole genome sequencing (WGS) or whole exome sequencing (WES) by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-C1, wherein the presence of at least one copy of HLA-C1 in the patient's genome identifies the patient as one who may benefit from treatment with a treatment regimen comprising a PD-1 axis binding antagonist.

In some aspects, the method further comprises determining whether the patient's genome comprises at least one copy of KIR2DL3.

In another aspect, the invention features a method of identifying a patient having NSCLC who may benefit from a treatment regimen comprising a PD-1 axis binding antagonist, the method comprising determining whether the patient's genome comprises at least one copy of HLA-C1 and at least one copy of KIR2DL3, wherein the presence of at least one copy of HLA-C1 and at least one copy of KIR2DL3 in the patient's genome identifies the patient as one who may benefit from treatment with a treatment regimen comprising a PD-1 axis binding antagonist.

In another aspect, the invention features a method of identifying a patient having NSCLC who may benefit from a treatment regimen comprising a PD-1 axis binding antagonist, the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-C1 and at least one copy of KIR2DL3, wherein the presence of at least one copy of HLA-C1 and at least one copy of KIR2DL3 in the patient's genome identifies the patient as one who may benefit from treatment with a treatment regimen comprising a PD-1 axis binding antagonist.

In another aspect, the invention features a method of identifying a patient having NSCLC who may benefit from a treatment regimen comprising a PD-1 axis binding antagonist, the method comprising determining whether the patient's genome comprises at least one copy of HLA-Bw4, wherein the presence of at least one copy of HLA-Bw4 in the patient's genome identifies the patient as one who may benefit from treatment with a treatment regimen comprising a PD-1 axis binding antagonist.

In another aspect, the invention features a method of identifying a patient having NSCLC who may benefit from a treatment regimen comprising a PD-1 axis binding antagonist, the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-Bw4, wherein the presence of at least one copy of HLA-Bw4 in the patient's genome identifies the patient as one who may benefit from treatment with a treatment regimen comprising a PD-1 axis binding antagonist.

In some aspects, the method further comprises determining whether the patient's genome comprises at least one copy of KIR3DL1.

In another aspect, the invention features a method of identifying a patient having NSCLC who may benefit from a treatment regimen comprising a PD-1 axis binding antagonist, the method comprising determining whether the patient's genome comprises at least one copy of HLA-Bw4 and at least one copy of KIR3DL1, wherein the presence of at least one copy of HLA-Bw4 and at least one copy of KIR3DL1 in the patient's genome identifies the patient as one who may benefit from treatment with a treatment regimen comprising a PD-1 axis binding antagonist.

In another aspect, the invention features a method of identifying a patient having NSCLC who may benefit from a treatment regimen comprising a PD-1 axis binding antagonist, the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-Bw4 and at least one copy of KIR3DL1, wherein the presence of at least one copy of HLA-Bw4 and at least one copy of KIR3DL1 in the patient's genome identifies the patient as one who may benefit from treatment with a treatment regimen comprising a PD-1 axis binding antagonist.

In another aspect, the invention features a method for selecting a therapy for a patient having NSCLC, the method comprising: (a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist; and (b) selecting a treatment regimen comprising a PD-1 axis binding antagonist based on the presence of at least one copy of HLA-C1 in the patient's genome.

In some aspects, the method further comprises determining whether the patient's genome comprises at least one copy of KIR2DL3.

In another aspect, the invention features a method for selecting a therapy for a patient having NSCLC, the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist; and (b) selecting a treatment regimen comprising a PD-1 axis binding antagonist based on the presence of at least one copy of HLA-C1 and at least one copy of KIR2DL3 in the patient's genome.

In another aspect, the invention features a method for selecting a therapy for a patient having NSCLC, the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist; and (b) selecting a treatment regimen comprising a PD-1 axis binding antagonist based on the presence of at least one copy of HLA-Bw4 in the patient's genome.

In some aspects, the method further comprises determining whether the patient's genome comprises at least one copy of KIR3DL1.

In another aspect, the invention features a method for selecting a therapy for a patient having NSCLC, the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist; and (b) selecting a treatment regimen comprising a PD-1 axis binding antagonist based on the presence of at least one copy of HLA-Bw4 and at least one copy of KIR3DL1 in the patient's genome.

In some aspects, the method further comprises administering an effective amount of a treatment regimen comprising a PD-1 axis binding antagonist to the patient.

In some aspects, the method further comprises presence of HLA-C1, HLA-Bw4, KIR2DL3, and/or KIR3DL1 in the patient's genome is determined using next-generation sequencing, Sanger sequencing, a polymerase chain reaction (PCR)-based assay, or a single nucleotide polymorphism (SNP) array.

In some aspects, the method further comprises next-generation sequencing comprises germline whole-genome sequencing or germline whole-exome sequencing.

In some aspects, the method further comprises PCR-based assay comprises quantitative PCR (qPCR), typing using sequence-specific primers (SSP), or typing using sequence specific oligonucleotide probes (SSO).

In another aspect, the invention features a method of treating NSCLC in a patient in need thereof who has been determined to have an increased level of natural killer (NK) cell infiltration in a tumor sample obtained from the patient relative to a reference level of NK cell infiltration, the method comprising administering to the patient an effective amount of a treatment regimen comprising a PD-1 axis binding antagonist.

In another aspect, the invention features a PD-1 axis binding antagonist for use in treating NSCLC in a patient in need thereof who has been determined to have an increased level of NK cell infiltration in a tumor sample obtained from the patient relative to a reference level of NK cell infiltration.

In another aspect, the invention features a method of treating NSCLC in a patient in need thereof, the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist; and (b) administering an effective amount of a treatment regimen comprising a PD-1 axis binding antagonist to the patient based on the increased level of NK cell infiltration in the tumor sample obtained from the patient relative to the reference level of NK cell infiltration.

In another aspect, the invention features a PD-1 axis binding antagonist for use in a method of treating NSCLC in a patient in need thereof, the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist; and (b) administering an effective amount of a treatment regimen comprising a PD-1 axis binding antagonist to the patient based on the increased level of NK cell infiltration in the tumor sample obtained from the patient relative to the reference level of NK cell infiltration.

In another aspect, the invention features a method of identifying a patient having NSCLC who may benefit from a treatment regimen comprising a PD-1 axis binding antagonist, the method comprising determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist.

In another aspect, the invention features a method of identifying a patient having NSCLC who may benefit from a treatment regimen comprising a PD-1 axis binding antagonist, the method comprising: (a) contacting a tumor sample obtained from the patient with one or more antibodies or nucleotide probes that bind to one or more NK cell markers to determine the level of NK cell infiltration in the tumor sample; and (b) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist.

In another aspect, the invention features a method for selecting a therapy for a patient having NSCLC, the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist; and (b) selecting a treatment regimen comprising a PD-1 axis binding antagonist based on the increased level of NK cell infiltration in the tumor sample obtained from the patient relative to the reference level of NK cell infiltration.

In some aspects, the method further comprises administering an effective amount of a treatment regimen comprising a PD-1 axis binding antagonist to the patient.

In some aspects, the level of NK cell infiltration is determined by determining an expression level of an NK cell gene signature or by counting a number of NK cells in the tumor sample.

In some aspects, the NK cell gene signature comprises one or more of the following genes: CD160, CD244, CTSW, FASLG, GZMA, GZMB, GZMH, IL18RAP, IL2RB, KIR2DL4, KLRB1, KLRC3, KLRD1, KRLF1, KLRK1, NCR1, NKG7, PRF1, XCL1, and XCL2.

In some aspects, the NK cell gene signature comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of the genes.

In some aspects, the reference level of NK cell infiltration is a median level.

In some aspects, the median level is a median level in a population of NSCLC patients.

In some aspects, the benefit is in terms of improved overall survival (OS) or improved progression-free survival (PFS).

In some aspects, the benefit is in terms of improved OS.

In some aspects, the benefit is in terms of improved PFS.

In some aspects, improvement is relative to treatment with a treatment regimen that does not comprise the PD-1 axis binding antagonist.

In some aspects, the NSCLC is non-squamous NSCLC or squamous NSCLC.

In some aspects, the NSCLC is non-squamous NSCLC.

In some aspects, the non-squamous NSCLC is metastatic non-squamous NSCLC.

In some aspects, the NSCLC is squamous NSCLC.

In some aspects, the squamous NSCLC is metastatic squamous NSCLC.

In some aspects, the patient is chemotherapy-naïve.

In some aspects, the treatment regimen is a first-line treatment regimen.

In some aspects, the PD-1 axis binding antagonist is selected from a PD-L1 binding antagonist, a PD-1 binding antagonist, and a PD-L2 binding antagonist.

In some aspects, the PD-1 axis binding antagonist is a PD-L1 binding antagonist.

In some aspects, the PD-L1 binding antagonist is an anti-PD-L1 antibody.

In some aspects, the anti-PD-L1 antibody comprises (a) a hypervariable region (HVR)-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In some aspects, the anti-PD-L1 antibody comprises (a) a VH comprising the amino acid sequence:

(SEQ ID NO: 9)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,

and (b) a VL comprising the amino acid sequence:

(SEQ ID NO: 10)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In some aspects, the anti-PD-L1 antibody is atezolizumab, durvalumab, avelumab, or MDX-1105.

In some aspects, the anti-PD-L1 antibody is atezolizumab.

In some aspects, the anti-PD-L1 antibody is administered intravenously or subcutaneously.

In some aspects, the atezolizumab is administered intravenously every two weeks at a dose of 840 mg.

In some aspects, the atezolizumab is administered intravenously every three weeks at a dose of 1200 mg.

In some aspects, the atezolizumab is administered intravenously every four weeks at a dose of 1680 mg.

In some aspects, the PD-1 axis binding antagonist is a PD-1 binding antagonist.

In some aspects, the PD-1 binding antagonist is an anti-PD-1 antibody.

In some aspects, the anti-PD-1 antibody is nivolumab, pembrolizumab, MEDI-0680, spartalizumab, cemiplimab, camrelizumab, sintilimab, tislelizumab, toripalimab, or dostarlimab.

In some aspects, the treatment regimen further comprises a taxane.

In some aspects, the taxane is nab-paclitaxel or paclitaxel.

In some aspects, the taxane is nab-paclitaxel.

In some aspects, the taxane is paclitaxel.

In some aspects, the treatment regimen further comprises a platinum-based chemotherapeutic agent.

In some aspects, the platinum-based chemotherapeutic agent is carboplatin.

In some aspects, the treatment regimen further comprises an anti-angiogenic agent.

In some aspects, the anti-angiogenic agent is an anti-VEGF antibody.

In some aspects, the anti-VEGF antibody is bevacizumab.

In some aspects, the NSCLC is metastatic non-squamous NSCLC, and the treatment regimen comprises atezolizumab, nab-paclitaxel, and carboplatin.

In some aspects, atezolizumab is administered as an intravenous (IV) infusion at a dose of 1200 mg on Day 1 of each 21-day cycle; nab-paclitaxel is administered as an IV infusion at a dose of 100 mg/m² on Days 1, 8, and 15 of each 21-day cycle; and carboplatin is administered at an area under the concentration curve (AUC) of 6 mg/mL/min on Day 1 of each 21-day cycle.

In some aspects, the NSCLC is metastatic non-squamous NSCLC, and the treatment regimen comprises atezolizumab, paclitaxel, and carboplatin.

In some aspects, atezolizumab is administered as an IV infusion at a dose of 1200 mg on Day 1 of each 21-day cycle; paclitaxel is administered as an IV infusion at a dose of 200 mg/m² on Day 1 each 21-day cycle; and carboplatin is administered at an AUC of 6 mg/mL/min on Day 1 of each 21-day cycle.

In some aspects, the NSCLC is metastatic non-squamous NSCLC, and the treatment regimen comprises atezolizumab, bevacizumab, paclitaxel, and carboplatin.

In some aspects, atezolizumab is administered as an IV infusion at a dose of 1200 mg on Day 1 of each 21-day cycle; bevacizumab is administered as an IV infusion at a dose of 15 mg/kg on Day 1 of each 21-day cycle; paclitaxel is administered as an IV infusion at a dose of 200 mg/m² on Day 1 each 21-day cycle; and carboplatin is administered at an AUC of 6 mg/mL/min on Day 1 of each 21-day cycle.

In some aspects, the NSCLC is metastatic squamous NSCLC, and the treatment regimen comprises atezolizumab, nab-paclitaxel, and carboplatin.

In some aspects, atezolizumab is administered as an IV infusion at a dose of 1200 mg on Day 1 of each 21-day cycle; nab-paclitaxel is administered as an IV infusion at a dose of 100 mg/m² on Days 1, 8, and 15 of each 21-day cycle; and carboplatin is administered at an area under the concentration curve (AUC) of 6 mg/mL/min on Day 1 of each 21-day cycle.

In some aspects, the NSCLC is metastatic squamous NSCLC, and the treatment regimen comprises atezolizumab, paclitaxel, and carboplatin.

In some aspects, atezolizumab is administered as an IV infusion at a dose of 1200 mg on Day 1 of each 21-day cycle; paclitaxel is administered as an IV infusion at a dose of 175 mg/m² or 200 mg/m2 on Days 1, 8, and 15 of each 21-day cycle; and carboplatin is administered at an AUC of 6 mg/mL/min on Day 1 of each 21-day cycle.

In some aspects, the method further comprises administering an additional therapeutic agent to the patient.

In some aspects, the additional therapeutic agent is selected from the group consisting of an immunotherapy agent, a cytotoxic agent, a growth inhibitory agent, a radiation therapy agent, an anti-angiogenic agent, and combinations thereof.

In another aspect, the invention features an article of manufacture comprising a PD-1 axis binding antagonist and instructions to administer the PD-1 axis binding antagonist for treatment of NSCLC in a patient in need thereof whose genome has been determined to comprise at least one copy of HLA-C1.

In another aspect, the invention features an article of manufacture comprising a PD-1 axis binding antagonist and instructions to administer the PD-1 axis binding antagonist for treatment of NSCLC in a patient in need thereof whose genome has been determined to comprise at least one copy of HLA-C1 and at least one copy of KIR2DL3.

In another aspect, the invention features an article of manufacture comprising a PD-1 axis binding antagonist and instructions to administer the PD-1 axis binding antagonist for treatment of NSCLC in a patient in need thereof whose genome has been determined to comprise at least one copy of HLA-Bw4.

In another aspect, the invention features an article of manufacture comprising a PD-1 axis binding antagonist and instructions to administer the PD-1 axis binding antagonist for treatment of treating NSCLC in a patient in need thereof whose genome has been determined to comprise at least one copy of HLA-Bw4 and at least one copy of KIR3DL1.

In another aspect, the invention features an article of manufacture comprising a PD-1 axis binding antagonist and instructions to administer the PD-1 axis binding antagonist for treatment of NSCLC in a patient in need thereof who has been determined to have an increased level of natural killer (NK) cell infiltration in a tumor sample obtained from the patient relative to a reference level of NK cell infiltration.

In another aspect, the invention features an article of manufacture comprising an NK cell-directed therapy agent and instructions to administer the NK cell-directed therapy agent for treatment of NSCLC in a patient in need thereof whose genome has been determined to lack KIR2DL3 or KIR3DL1.

### BRIEF DESCRIPTION OF THE DRAWINGS

FIG. 1A is a plot showing the overall survival (OS) hazard ratio (HR) for non-small cell lung cancer (NSCLC) patients who were carriers of at least one copy of each of the human leukocyte antigen (HLA) allele HLA-C1 and the killer-cell immunoglobulin-like receptor (KIR) gene KIR2DL3 and were treated with a therapy comprising atezolizumab compared to a control in the IMpower130, IMpower131, or IMpower150 clinical trial. Estimate of Treatment Effect (TE), Standard Error of TE (seTE), P-value, and weight (fixed and random) are shown. Fixed effect and random effect models are shown.

FIG. 1B is a plot showing the progression-free survival (PFS) HR for NSCLC patients who were carriers of at least one copy of each of HLA-C1 and KIR2DL3 and were treated with a therapy comprising atezolizumab compared to a control in the IMpower130, IMpower131, or IMpower150 clinical trial.

FIG. 2A is a plot showing the OS HR for NSCLC patients who were carriers of at least one copy of each of the HLA allele HLA-Bw4 and the KIR gene KIR3DL1 and were treated with a therapy comprising atezolizumab compared to a control in the IMpower130, IMpower131, or IMpower150 clinical trial.

FIG. 2B is a plot showing the PFS HR for NSCLC patients who were carriers of at least one copy of each of HLA-Bw4 and KIR3DL1 and were treated with a therapy comprising atezolizumab compared to a control in the IMpower130, IMpower131, or IMpower150 clinical trial.

FIG. 3A is a plot showing the OS HR for NSCLC patients who were carriers of at least one copy of HLA-C1 and were treated with a therapy comprising atezolizumab compared to a control in the IMpower130, IMpower131, or IMpower150 clinical trial.

FIG. 3B is a plot showing the PFS HR for NSCLC patients who were carriers of at least one copy of HLA-C1 and were treated with a therapy comprising atezolizumab compared to a control in the IMpower130, IMpower131, or IMpower150 clinical trial.

FIG. 4A is a plot showing the OS HR for NSCLC patients who were carriers of at least one copy of HLA-Bw4 and were treated with a therapy comprising atezolizumab compared to a control in the IMpower130, IMpower131, or IMpower150 clinical trial.

FIG. 4B is a plot showing the PFS HR for NSCLC patients who were carriers of at least one copy of HLA-Bw4 and were treated with a therapy comprising atezolizumab compared to a control in the IMpower130, IMpower131, or IMpower150 clinical trial.

FIG. 5A is a plot showing the OS HR for NSCLC and melanoma patients who were carriers of at least one copy of HLA-Bw4 and were treated with a therapy comprising an immune checkpoint blockade (ICB) compared to a control in the Chowell et al. data (see Example 1d) or MSK-IMPACT (see, e.g., Zehir et al. Nat. Med. 23:703-713, 2017).

FIG. 5B is a plot showing the OS HR for NSCLC and melanoma patients who were carriers of at least one copy of HLA-C1 and were treated with a therapy comprising an immune checkpoint blockade (ICB) compared to a control in the Chowell et al. data (see Example 1d) or MSK-IMPACT.

FIG. 6A is a plot showing the OS HR for NSCLC patients who had an above-median natural killer (NK) cell score and were treated with a therapy comprising atezolizumab compared to a control in the IMpower131 or IMpower150 clinical trial.

FIG. 6B is a plot showing the OS HR for NSCLC patients who had an above-median NK cell score and were treated with a therapy comprising atezolizumab compared to a control in the IMpower131 or IMpower150 clinical trial.

FIG. 7A is a plot showing the OS HR for patients who had an above-median NK cell score and were treated with a therapy comprising atezolizumab compared to a control in the listed clinical trials.

FIG. 7B is a plot showing the OS HR for patients who had an above-median CD8A level and were treated with a therapy comprising atezolizumab compared to a control in the listed clinical trials.

FIG. 8A is a plot showing the OS HR for renal cell carcinoma (RCC) patients who were carriers of at least one copy of each of HLA-C1 and KIR2DL3 and were treated with a therapy comprising atezolizumab compared to a control in the IMmotion151 clinical trial.

FIG. 8B is a plot showing the PFS HR for RCC patients who were carriers of at least one copy of each of HLA-C1 and KIR2DL3 and were treated with a therapy comprising atezolizumab compared to a control in the IMmotion151 clinical trial.

FIG. 9A is a plot showing the OS HR for RCC patients who were carriers of at least one copy of each of HLA-Bw4 and KIR3DL1 and were treated with a therapy comprising atezolizumab compared to a control in the IMmotion151 clinical trial.

FIG. 9B is a plot showing the PFS HR for RCC patients who were carriers of at least one copy of each of HLA-Bw4 and KIR3DL1 and were treated with a therapy comprising atezolizumab compared to a control in the IMmotion151 clinical trial.

FIG. 10A is a plot showing the OS HR for RCC patients who were carriers of at least one copy of HLA-C1 and were treated with a therapy comprising atezolizumab compared to a control in the IMmotion151 clinical trial.

FIG. 10B is a plot showing the PFS HR for RCC patients who were carriers of at least one copy of HLA-C1 and were treated with a therapy comprising atezolizumab compared to a control in the IMmotion151 clinical trial.

FIG. 11A is a plot showing the OS HR for RCC patients who were carriers of at least one copy of HLA-Bw4 and were treated with a therapy comprising atezolizumab compared to a control in the IMmotion151 clinical trial.

FIG. 11B is a plot showing the PFS HR for RCC patients who were carriers of at least one copy of HLA-Bw4 and were treated with a therapy comprising atezolizumab compared to a control in the IMmotion151 clinical trial.

FIG. 12A is a plot showing the OS HR for RCC patients who had an above-median NK cell score and were treated with a therapy comprising atezolizumab compared to a control in the IMmotion150 or IMmotion151 clinical trial.

FIG. 12B is a plot showing the PFS HR for RCC patients who had an above-median NK cell score and were treated with a therapy comprising atezolizumab compared to a control in the IMmotion150 or IMmotion151 clinical trial.

FIG. 13A is a plot showing the OS HR for RCC patients who had an above-median CD8A level and were treated with a therapy comprising atezolizumab compared to a control in the IMmotion150 or IMmotion151 clinical trial.

FIG. 13B is a plot showing the PFS HR for RCC patients who had an above-median CD8A level and were treated with a therapy comprising atezolizumab compared to a control in the IMmotion150 or IMmotion151 clinical trial.

FIG. 14 is a plot showing the risk of immune checkpoint inhibitor (ICI)-associated pneumonitis in patients who carried the HLA class II allele HLA-DRB1*15:01 and were treated with an ICI compared to a control in the indicated cohorts. GNE, Genentech; PICI, Parker Institute for Cancer Immunotherapy; PMC, Peter MacCallum Cancer Centre; OR, odds ratio; CI, confidence interval; W, weight.

FIG. 15 is a plot showing OS HR for the indicated cohorts showing that HLA class I loss of heterozygosity (LOH) is not associated with outcome. The plot shows any class I LOH versus no LOH for the atezolizumab arms of the indicated studies.

FIG. 16 is a series of plots showing that TMB does not modify the impact of LOH on outcome.

FIG. 17 is a plot showing that class I LOH is associated with lower CD8A expression. The plot shows any class I LOH versus no LOH for the atezolizumab arms of the indicated studies.

FIG. 18 is a plot showing OS HR for the indicated cohorts showing that HLA class II LOH is associated with poor outcome. The plot shows any class II LOH versus no LOH for the atezolizumab arms of the indicated studies.

### DETAILED DESCRIPTION OF THE INVENTION

The present invention provides therapeutic and diagnostic methods and compositions for cancer, for example, lung cancer (e.g., NSCLC (e.g., non-squamous NSCLC or squamous NSCLC)) or renal cancer (e.g., RCC). The present invention is based, at least in part, on the discovery described herein that the presence of particular human leukocyte antigen genes (e.g., HLA-C1 or HLA-Bw4) and/or killer-cell immunoglobulin-like receptor genes (e.g., KIR2DL3 or KIR3DL1) in a patient's genome is associated with improved treatment benefit from a treatment regimen that includes a PD-1 axis binding antagonist (e.g., atezolizumab). The invention is also based, at least in part, on the discovery described herein that elevated NK cell infiltration in a tumor sample obtained from a patient is associated with improved treatment benefit from a treatment regimen that includes a PD-1 axis binding antagonist (e.g., atezolizumab). The invention is also based, at least in part, on the discovery described herein that patients whose genome lacks one or more of KIR2DL3 or KIR3DL1 may benefit from a treatment regimen that includes an NK cell-directed therapy agent.

### I. Definitions

The following abbreviations are used herein:

| | | | |
|---|---|---|---|
| CAS | Chemical Abstracts Service | OS | overall survival |
| CDR | complementarity determining region | PCR | polymerase chain reaction |
| CR | complete response | PD-1 | programmed death 1 |
| DNA | deoxyribonucleic acid | PD-L1 | programmed death ligand 1 |
| DOR | duration of response | PD-L2 | programmed death ligand 2 |
| Fab | fragment antigen-binding | PFS | progression-free survival |
| Fc | fragment crystallizable | PR | partial response |
| FFPE | formalin-fixed and paraffin-embedded | qPCR | quantitative PCR |
| FR | framework | RCC | renal cell carcinoma |
| HLA | human leukocyte antigen | RNA | ribonucleic acid |
| HVR | hypervariable region | SLD | sum of the longest diameters |
| IHC | immunohistochemistry | SNP | single nucleotide polymorphism |
| KIR | killer-cell immunoglobulin-like receptor | SSP | sequence-specific primers |
| NK | natural killer | SSO | sequence-specific oligonucleotide probes |
| ORR | overall response rate/objective response rate | | |

The term “human leukocyte antigen C” and “HLA-C” refers to an HLA class I heavy chain gene. Class I molecules play a central role in the immune system by presenting peptides derived from cytosolic proteins, and are expressed in nearly all cells. The HLA-C receptor is a heterodimer that includes a mature HLA-C gene product heavy chain and a β2-microglobulin light chain. The heavy chain is approximately 45 kDa and its gene contains 8 exons. Typically, exon 1 encodes the leader peptide, exons 2 and 3 encode the alpha-1 and alpha-2 domains, which both bind the peptide, exon 4 encodes the alpha-3 domain, exon 5 encodes the transmembrane region, and exons 6 and 7 encode the cytoplasmic tail. Polymorphisms within exon 2 and exon 3 are generally responsible for the peptide binding specificity of each class I molecule. Approximately 6,600 HLA-C alleles have been described. HLA-C alleles fall within the HLA-C1 and HLA-C2 groups. Additional information regarding HLA-C may be found, e.g., under UniProt Accession No. P10321.

The term “HLA-C1” refers to an HLA-C gene allele group typically characterized by an asparagine (Asn) residue at position 80 of the alpha-1 domain. Exemplary HLA-C1 alleles include, but are not limited to, Cw*0102, Cw*0103, Cw*0104, Cw*0105, Cw*0302, Cw*0303, Cw*0304, Cw*0305, Cw*0306, Cw*0308, Cw*0309, Cw*0310, Cw*0311, Cw*0312, Cw*0313, Cw*0314, Cw*0701, Cw*0702, Cw*0703, Cw*0704, Cw*0705, Cw*0706, Cw*0708, Cw*0710, Cw*0711, Cw*0712, Cw*0713, Cw*0714, Cw*0715, Cw*0801, Cw*0802, Cw*0803, Cw*0804, Cw*0805, Cw*0806, Cw*0807, Cw*0808, Cw*0809, Cw*1202, Cw*1203, Cw*1206, Cw*1208, Cw*1301, Cw*1402, Cw*1403, Cw*1405, Cw*1507, Cw*1601, and Cw*1604. Other HLA-C1 alleles are known in the art. See, e.g., the IPD-IMGT/HLA database (ebi.ac.uk/ipd/imgt/hla).

The term “HLA-C2” refer to an HLA-C gene allele group typically characterized by a lysine (Lys) residue at position 80 of the alpha-1 domain. Exemplary HLA-C2 alleles include, but are not limited to, Cw*0202, Cw*0203, Cw*0204, Cw*0205, Cw*0307, Cw*0401, Cw*0403, Cw*0404, Cw*0405, Cw*0406, Cw*0407, Cw*0408, Cw*0501, Cw*0502, Cw*0503, Cw*0504, Cw*0602, Cw*0603, Cw*0604, Cw*0605, Cw*0606, Cw*0607, Cw*0707, Cw*0709, Cw*1204, Cw*1205, Cw*1207, Cw*1404, Cw*1502, Cw*1503, Cw*1504, Cw*1505, Cw*1506, Cw*1508, Cw*1509, Cw*1510, Cw*1511, Cw*1602, Cw*1701, Cw*1702, Cw*1703, Cw*1801, and Cw*1802. Other HLA-C2 alleles are known in the art. See, e.g., the IPD-IMGT/HLA database.

The term “HLA-B” refers to an HLA class I heavy chain gene. The HLA-B receptor is a heterodimer that includes a mature HLA-B gene product heavy chain and a 32-microglobulin light chain. The heavy chain is approximately 45 kDa and its gene contains 8 exons. Typically, exon 1 encodes the leader peptide, exons 2 and 3 encode the alpha-1 and alpha-2 domains, which both bind the peptide, exon 4 encodes the alpha-3 domain, exon 5 encodes the transmembrane region, and exons 6 and 7 encode the cytoplasmic tail. Polymorphisms within exon 2 and exon 3 are generally responsible for the peptide binding specificity of each class I molecule. Additional information regarding HLA-B may be found, e.g., under UniProt Accession No. P01889. Either the Bw4 or the Bw6 epitope is expressed by virtually all HLA-B molecules; Bw4 also is found on some HLA-A proteins (e.g., HLA-A*24:02, -A*32:01, and -A*23:01).

The term “HLA-Bw4” refers to a class I HLA allele group characterized by a Bw4 epitope within the alpha-1 helix. The Bw4 epitope is typically defined by five residues within the alpha-1 helix (i.e., positions 77, 80, 81, 82, and 83), which serologically distinguish it from the Bw6 epitope. HLA-Bw4 molecules such as HLA-B*57:01 or HLA-B*15:13 are characterized by the presence of Asn77, Ile80, Ala81, Leu82, and Arg83. Although residues 82 and 83 of the Bw4 sequence are conserved, the remaining residues may vary to create up to eight different Bw4 motifs. As used herein, the term includes HLA-B or HLA-A molecules that include a Bw4 epitope.

The term “killer cell immunoglobulin-like receptor 2DL3” and “KIR2DL3” refers to any native KIR2DL3 from any vertebrate source, including mammals such as primates (e.g., humans) and rodents (e.g., mice and rats), unless otherwise indicated. The term encompasses “full-length,” unprocessed KIR2DL3 as well as any form of KIR2DL3 that results from processing in the cell. The term also encompasses naturally occurring variants of KIR2DL3, e.g., splice variants or allelic variants. KIR2DL3 is an inhibitory KIR gene that recognizes HLA-C molecules (e.g., HLA-C1 molecules) and certain HLA-B molecules (see, e.g., Pende et al. Front. Immunol. doi: 10.3389/fimmu.2019.01179, 2019). KIR2DL3 is also known in the art as CD158 antigen-like family member B2, KIR-023 GB, Killer inhibitory receptor cl 2-3, NKAT2a, NKAT2b, Natural killer-associated transcript 2, p58 natural killer cell receptor clone CL-6, p58.2 MHC class-I-specific NK receptor, and CD158b2. Additional information about human KIR2DL3 is found under NCBI Gene ID: 3804. The nucleic acid sequence of an exemplary human KIR2DL3 is shown under NCBI Reference Sequence: NM_015868.3. The amino acid sequence encoded by an exemplary human KIR2DL3 gene is shown under UniProt Accession No. P43628-1.

The term “killer cell immunoglobulin-like receptor 3DL1” and “KIR3DL1” refers to any native KIR3DL1 from any vertebrate source, including mammals such as primates (e.g., humans) and rodents (e.g., mice and rats), unless otherwise indicated. The term encompasses “full-length,” unprocessed KIR3DL1 as well as any form of KIR3DL1 t hat results from processing in the cell. The term also encompasses naturally occurring variants of KIR3DL1, e.g., splice variants or allelic variants. KIR3DL1 is an inhibitory KIR gene that recognizes HLA-B molecules (e.g., HLA-Bw4 molecules) and also some HLA-A Bw4 bearing allotypes. KIR3DL1 is also known in the art as CD158 antigen-like family member E, HLA-BW4-specific inhibitory NK cell receptor, natural killer-associated transcript 3 (NKAT-3), p70 natural killer cell receptor clones CL-2/CL-11, and CD158e. Additional information about human KIR3DL1 is found under NCBI Gene ID: 3811. The nucleic acid sequence of an exemplary human KIR3DL1 is shown under NCBI Reference Sequence: NM_013289.3. The amino acid sequence encoded by an exemplary human KIR3DL1 gene is shown under UniProt Accession No. P43629-1.

The term “natural killer cell” and “NK cell” refers to a type of lymphocyte of the innate immune system that can detect and eliminate, e.g., cancer cells. NK cells include, e.g., CD56^{bright} (also referred to as CD56^{high}) cells, which constitute the majority of NK cells, and are found in bone marrow, secondary lymphoid tissue, liver and skin, and CD56^{dim} (also referred to as CD56^{low}) cells, which are primarily found in the peripheral blood system, and are characterized by cytotoxic ability. CD56^{dim} NK cells are typically CD16 positive and may be referred to as CD56^{dim} CD16^{bright} NK cells; CD56^{bright} cells can transition into CD56^{dim} cells by acquiring CD16.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

The terms “programmed death ligand 1” and “PD-L1” refer herein to native sequence human PD-L1 polypeptide. Native sequence PD-L1 polypeptides are provided under Uniprot Accesion No. Q9NZQ7. For example, the native sequence PD-L1 may have the amino acid sequence as set forth in Uniprot Accession No. Q9NZQ7-1 (isoform 1). In another example, the native sequence PD-L1 may have the amino acid sequence as set forth in Uniprot Accession No. Q9NZQ7-2 (isoform 2). In yet another example, the native sequence PD-L1 may have the amino acid sequence as set forth in Uniprot Accession No. Q9NZQ7-3 (isoform 3). PD-L1 is also referred to in the art as “programmed cell death 1 ligand 1,” “PDCD1 LG1,” “CD274,” “B7-H,” and “PDL1.”

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

For the purposes herein, “atezolizumab” is an Fc-engineered, humanized, non-glycosylated IgG1 kappa immunoglobulin that binds PD-L1 and comprises the heavy chain sequence of SEQ ID NO: 1 and the light chain sequence of SEQ ID NO: 2. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances), Proposed INN: List 112, Vol. 28, No. 4, published Jan. 16, 2015 (see page 485).

The terms “cancer” and “cancerous” refer to or describe the physiological condition in mammals that is typically characterized by unregulated cell growth. Aspects of cancer include solid tumor cancers and non-solid tumor cancers. Examples of cancer include but are not limited to, carcinoma, lymphoma, blastoma, sarcoma, and leukemia or lymphoid malignancies. More particular examples of such cancers include, but are not limited to, bladder cancer (e.g., urothelial carcinoma (UC), including metastatic UC (mUC); muscle-invasive bladder cancer (MIBC), and non-muscle-invasive bladder cancer (NMIBC)); kidney or renal cancer (e.g., renal cell carcinoma (RCC)); lung cancer, including small-cell lung cancer, non-small cell lung cancer, adenocarcinoma of the lung, and squamous carcinoma of the lung; cancer of the urinary tract; breast cancer (e.g., HER2+ breast cancer and triple-negative breast cancer (TNBC), which are estrogen receptors (ER−), progesterone receptors (PR−), and HER2 (HER2−) negative); prostate cancer, such as castration-resistant prostate cancer (CRPC); cancer of the peritoneum; hepatocellular cancer; gastric or stomach cancer, including gastrointestinal cancer and gastrointestinal stromal cancer; pancreatic cancer (e.g., pancreatic ductal adenocarcinoma (PDAC)); glioblastoma; cervical cancer; ovarian cancer; liver cancer (e.g., hepatocellular carcinoma (HCC)); hepatoma; colon cancer; rectal cancer; colorectal cancer; endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo malignant melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases. In one instance, the cancer is lung cancer (e.g., NSCLC, e.g., non-squamous NSCLC or squamous NSCLC). In another instance, the cancer is renal cancer (e.g., RCC)). The cancer may be locally advanced or metastatic. In some instances, the cancer is locally advanced. In other instances, the cancer is metastatic. In some instances, the cancer is stage IV cancer. In some instances, the cancer may be unresectable (e.g., unresectable locally advanced or metastatic cancer).

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “cell proliferative disorder” and “proliferative disorder” refer to disorders that are associated with some degree of abnormal cell proliferation. In one embodiment, the cell proliferative disorder is cancer. In another embodiment, the cell proliferative disorder is a tumor.

The terms “B cell proliferative disorder” or “B cell malignancy” refer to disorders that are associated with some degree of abnormal B cell proliferation and include, for example, lymphomas, leukemias, myelomas, and myelodysplastic syndromes. In one embodiment, the B cell proliferative disorder is a lymphoma, such as non-Hodgkin's lymphoma (NHL), including, for example, DLBCL (e.g., relapsed or refractory DLBCL), FL (e.g., relapsed or refractory FL or transformed FL), or MCL. In another embodiment, the B cell proliferative disorder is a leukemia, such as chronic lymphocytic leukemia (CLL). In yet another embodiment, the B cell proliferative disorder is a central nervous system lymphoma (CNSL).

As used herein, “treating” comprises effective cancer treatment with an effective amount of a therapeutic agent (e.g., a PD-1 axis binding antagonist (e.g., atezolizumab) or an NK cell-directed therapy agent) or combination of therapeutic agents (e.g., a PD-1 axis antagonist and one or more additional therapeutic agents, e.g., a taxane (e.g., nab-paclitaxel or paclitaxel), a platinum-based chemotherapeutic agent (e.g., carboplatin), an anti-angiogenic agent (e.g., an anti-VEGF antibody such as bevacizumab), and/or a NK cell-directed therapy agent). Treating herein includes, inter alia, adjuvant therapy, neoadjuvant therapy, non-metastatic cancer therapy (e.g., locally advanced cancer therapy), and metastatic cancer therapy. The treatment may be first-line treatment (e.g., the patient may be previously untreated or not have received prior systemic therapy), or second line or later treatment.

Herein, an “effective amount” refers to the amount of a therapeutic agent (e.g., a PD-1 axis binding antagonist (e.g., atezolizumab) or an NK cell-directed therapy agent) or a combination of therapeutic agents (e.g., a PD-1 axis antagonist and one or more additional therapeutic agents, e.g., a taxane (e.g., nab-paclitaxel or paclitaxel), a platinum-based chemotherapeutic agent (e.g., carboplatin), an anti-angiogenic agent (e.g., bevacizumab), and/or a NK cell-directed therapy agent)), that achieves a therapeutic result. In some examples, the effective amount of a therapeutic agent or a combination of therapeutic agents is the amount of the agent or of the combination of agents that achieves a clinical endpoint of improved survival (e.g., disease-free survival (DFS), progression-free survival (PFS) and/or overall survival (OS)), improved overall response rate (ORR), a complete response (CR), a pathological complete response (pCR), a partial response (PR), and/or improved duration of response (DOR). Improvement (e.g., in terms of response rate (e.g., ORR, CR, and/or PR), survival (e.g., PFS and/or OS), or DOR) may be relative to a suitable reference treatment, for example, treatment that does not include the PD-1 axis binding antagonist and/or treatment that does not include the taxane (e.g., nab-paclitaxel or paclitaxel), platinum-based chemotherapeutic agent (e.g., carboplatin), anti-angiogenic agent (e.g., bevacizumab), and/or NK cell-directed therapy agent. In some aspects, improvement may be relative to treatment with a treatment regimen that does not include the PD-1 axis binding antagonist.

As used herein, “complete response” and “CR” refers to disappearance of all target lesions.

As used herein, “partial response” and “PR” refers to at least a 30% decrease in the sum of the longest diameters (SLD) of target lesions, taking as reference the baseline SLD prior to treatment.

As used herein, “overall response rate,” “objective response rate,” and “ORR” refer interchangeably to the sum of CR rate and PR rate.

As used herein, “progression-free survival” and “PFS” refer to the length of time during and after treatment during which the cancer does not get worse. PFS may include the amount of time patients have experienced a CR or a PR, as well as the amount of time patients have experienced stable disease.

In some examples, PFS may be determined using Response Evaluation Criteria in Solid Tumors (RECIST) version 1.1. For example, in some instances, PFS is defined as the time between the date of randomization and the date of first documented disease progression or death, whichever occurs first.

As used herein, “overall survival” and “OS” refer to the length of time from either the date of diagnosis or the start of treatment for a disease (e.g., cancer) that the patient is still alive. For example, in some instances, OS is defined as the time between the date of randomization and date of death from any cause.

As used herein, the term “duration of response” and “DOR” refer to a length of time from documentation of a tumor response until disease progression or death from any cause, whichever occurs first.

As used herein, the term “chemotherapeutic agent” refers to a compound useful in the treatment of cancer (e.g., NSCLC). Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1 C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, prednisone, dacarbazine, mechlorethamine, cisplatin, methotrexate, 5-fluorouracil, and ara-C); and (x) pharmaceutically acceptable salts, acids, prodrugs, and derivatives of any of the above.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy)quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

A “taxane” as used herein is an agent (e.g., a diterpene) which may bind to tubulin, promoting microtubule assembly and stabilization and/or prevent microtubule depolymerization. Exemplary taxanes include, but are not limited to, paclitaxel (i.e., TAXOL®, CAS #33069-62-4), docetaxel (i.e., TAXOTERE®, CAS #114977-28-5), larotaxel, cabazitaxel, milataxel, tesetaxel, and/or orataxel. Taxanes included herein also include taxoid 10-deacetylbaccatin III and/or derivatives thereof. In some embodiments, the taxane is an albumin-coated nanoparticle (e.g., nano-albumin bound (nab)-paclitaxel, i.e., ABRAXANE® and/or nab-docetaxel, ABI-008). In some embodiments, the taxane is nab-paclitaxel (ABRAXANE®). In some embodiments, the taxane is formulated in CREMAPHOR® (e.g., TAXOL®) and/or in TWEEN® such as polysorbate 80 (e.g., TAXOTERE®). In some embodiments, the taxane is liposome-encapsulated taxane. In some embodiments, the taxane is a prodrug form and/or conjugated form of taxane (e.g., DHA covalently conjugated to paclitaxel, paclitaxel poliglumex, and/or linoleyl carbonate-paclitaxel). In some embodiments, the paclitaxel is formulated with substantially no surfactant (e.g., in the absence of CREMAPHOR® and/or TWEEN®, such as TOCOSOL® paclitaxel).

Chemotherapeutic agents also include “platinum-based” chemotherapeutic agents, which comprise an organic compound which contains platinum as an integral part of the molecule. Typically, platinum-based chemotherapeutic agents are coordination complexes of platinum. Platinum-based chemotherapeutic agents are sometimes called “platins” in the art. Examples of platinum-based chemotherapeutic agents include, but are not limited to, cisplatin, carboplatin, oxaliplatin, nedaplatin, triplatin tetranitrate, phenanthriplatin, picoplatin, lipoplatin, and satraplatin. Platinum-based chemotherapeutic agents (e.g., cisplatin or carboplatin) may be administered in combination with one or more additional chemotherapeutic agents, e.g., a nucleoside analog (e.g., gemcitabine).

A “platinum-based chemotherapy,” as used herein, refers to a chemotherapy regimen that includes a platinum-based chemotherapeutic agent. For example, a platinum-based chemotherapy may include a platinum-based chemotherapeutic agent (e.g., cisplatin or carboplatin) in combination with one or more additional chemotherapeutic agents, e.g., a nucleoside analog (e.g., gemcitabine).

An “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent as defined above, e.g., antibodies to VEGF-A or the VEGF-A receptor (e.g., KDR receptor or Flt-1 receptor), anti-PDGFR inhibitors such as GLEEVEC™ (imatinib mesylate). Anti-angiogenesis agents also include native angiogenesis inhibitors, e.g., angiostatin, endostatin, etc. See, for example, Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12):1359-1364 (1999); Tonini et al., Oncogene, 22:6549-6556 (2003) and Sato Int. J. Clin. Oncol., 8:200-206 (2003).

An “anti-VEGF antibody” is an antibody that binds to VEGF with sufficient affinity and specificity. In certain embodiments, the antibody will have a sufficiently high binding affinity for VEGF, for example, the antibody may bind hVEGF with a Kd value of between 100 nM-1 pM. Antibody affinities may be determined, e.g., by a surface plasmon resonance-based assay (such as the BIAcore® assay as described in PCT Application Publication No. WO2005/012359); enzyme-linked immunoabsorbent assay (ELISA); and competition assays (e.g. radioimmunoassays (RIAs)).

In certain embodiments, the anti-VEGF antibody can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as PIGF, PDGF, or bFGF. In one embodiment, anti-VEGF antibody is a monoclonal antibody that binds to the same epitope as the monoclonal anti-VEGF antibody A4.6.1 produced by hybridoma ATCC HB 10709. In another embodiment, the anti-VEGF antibody is a recombinant humanized anti-VEGF monoclonal antibody generated according to Presta et al. (Cancer Res. 57:4593-4599, 1997), including but not limited to the antibody known as bevacizumab (BV; AVASTIN®).

The anti-VEGF antibody “bevacizumab,” also known as “rhuMAb VEGF,” “BV,” or “AVASTIN®,” is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 Daltons and is glycosylated. Bevacizumab and other humanized anti-VEGF antibodies are further described in U.S. Pat. No. 6,884,879, issued Feb. 26, 2005, the entire disclosure of which is expressly incorporated herein by reference. Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., (Journal of Immunological Methods 288:149-164, 2004). Other preferred antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183, and Q89.

The term “NK cell-directed therapy agent” refers to an agent that includes NK cells or that modulates the number, activity, or function of NK cells. In some instances, the NK cell-directed therapy agent comprises adoptive cell transfer (e.g., with allogeneic NK cells, autologous NK cells, off-the-shelf NK cells, or chimeric antigen receptor (CAR)-NK cells), cytokine therapy, an NK cell engager (e.g., a bispecific killer cell engager (BiKE), a tri-specific killer cell engager (TriKE), or a tetra-specific killer cell engager (TetraKE)), an NK cell checkpoint receptor antagonist, or an oncolytic virus. Exemplary NK cell-directed therapy agents are described, e.g., in Hodgins et al. J. Clin. Invest. 129(9):3499-3510, 2019.

The term “NK cell engager” refers to a molecule that brings together an NK cell and a tumor cell, e.g., by binding to one or more targets (e.g., proteins, e.g., receptors) on the surface of an NK cell (e.g., CD16, NKG2D, a SLAM family protein, NKp30, NKp44, or NKp46) and one or more targets (e.g., proteins, e.g., receptors) on the surface of the tumor cell (e.g., a tumor antigen, including CD30, CD33, EGFR, BCMA, or any tumor antigen described in Table C). An NK cell engager may be multispecific, e.g., bispecific, trispecific, or tetraspecific. An NK cell engager may be multivalent for a particular target, e.g., bivalent, trivalent, tetravalent, pentavalent, or hexavalent. For example, an NK cell engager may be at least bivalent for CD16A, i.e., comprise at least two CD16A antigen-binding moieties. In some examples, an NK cell engager includes at least a first targeting domain binding to an epitope on a NK cell and at least a second targeting domain binding to a different target, e.g., a tumor antigen (e.g., any tumor antigen described in Table C). Exemplary NK cell engagers are described, e.g., in WO 2019/198051; Reusch et al., mAbs, 6(3):727-738; 2014; U.S. Pat. No. 7,129,330B1; U.S. Pat. No. 9,035,026B2; WO0111059A1; Treder et al., Journal of Clinical Oncology, 34(15 suppl), 2016; and Ellwanger et al., J Immunother Cancer, 3(Suppl 2): 219, 2015. In some embodiments, the NK cell engager is a nanoparticle-based NK cell engager, e.g., a nanoparticle-based trispecific NK cell engager (nano-TriNKE); see, e.g., Au et al. Science Advances 6(27):eaba8564, 2020. Exemplary NK cell engagers include, e.g., IPH6101 (Innate Pharma/Sanofi).

The term “patient” refers to a human patient. For example, the patient may be an adult.

The term “antibody” herein specifically covers monoclonal antibodies (including full-length monoclonal antibodies), polyclonal antibodies, multispecific antibodies (e.g., bispecific antibodies), and antibody fragments so long as they exhibit the desired biological activity. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms refer to an antibody comprising an Fc region.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447). Therefore, the C-terminal lysine (Lys447), or the C-terminal glycine (Gly446) and lysine (Lys447), of the Fc region may or may not be present. Amino acid sequences of heavy chains including an Fc region are denoted herein without the C-terminal lysine (Lys447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one embodiment, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

A “naked antibody” refers to an antibody that is not conjugated to a heterologous moiety (e.g., a cytotoxic moiety) or radiolabel. The naked antibody may be present in a pharmaceutical composition.

“Antibody fragments” comprise a portion of an intact antibody, preferably comprising the antigen-binding region thereof. In some instances, the antibody fragment described herein is an antigen-binding fragment. Examples of antibody fragments include Fab, Fab′, F(ab′)₂, and Fv fragments; diabodies; linear antibodies; single-chain antibody molecules (e.g., scFvs); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)). Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “in combination with” refers to administration of one treatment modality in addition to another treatment modality, for example, a treatment regimen that includes administration of a PD-1 axis binding antagonist (e.g., atezolizumab) and a taxane (e.g., nab-paclitaxel or paclitaxel), a platinum-based chemotherapeutic agent (e.g., carboplatin), an anti-angiogenic agent (e.g., an anti-VEGF antibody such as bevacizumab), and/or a NK cell-directed therapy agent. As such, “in combination with” refers to administration of one treatment modality before, during, or after administration of the other treatment modality to the patient.

A drug that is administered “concurrently” with one or more other drugs is administered during the same treatment cycle, on the same day of treatment, as the one or more other drugs, and, optionally, at the same time as the one or more other drugs. For instance, for cancer therapies given every 3 weeks, the concurrently administered drugs are each administered on day 1 of a 3 week cycle. The term “detection” includes any means of detecting, including direct and indirect detection.

The term “biomarker” as used herein refers to an indicator, e.g., predictive, diagnostic, and/or prognostic, which can be detected in a sample, for example, an HLA gene (e.g., HLA-C1 or HLA-Bw4), a KIR gene (e.g., KIR2DL3 or KIR3DL1), NK cell infiltration, or an NK cell signature (e.g., an NK cell signature that includes one or more of the following genes: CD160, CD244, CTSW, FASLG, GZMA, GZMB, GZMH, IL18RAP, IL2RB, KIR2DL4, KLRB1, KLRC3, KLRD1, KRLF1, KLRK1, NCR1, NKG7, PRF1, XCL1, and XCL2). The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers.

The term “CD160” as used herein, refers to any native CD160 (Cluster of Differentiation 160; also known as NK1; BY55; and NK28) from any vertebrate source, including mammals such as primates (e.g., humans) and rodents (e.g., mice and rats), unless otherwise indicated. The term encompasses “full-length,” unprocessed CD160 as well as any form of CD160 that results from processing in the cell. The term also encompasses naturally occurring variants of CD160, e.g., splice variants or allelic variants. The nucleic acid sequence of an exemplary human CD160 is shown under NCBI Reference Sequence: NM_007053.4. The amino acid sequence of an exemplary protein encoded by human CD160 is shown under UniProt Accession No. Q6FH89.

The term “CD244” as used herein, refers to any native CD244 (Cluster of Differentiation 244; also known as Natural Killer Cell Receptor 2B4; NAIL; NKR2B4; Nmrk; and SLAMF4) from any vertebrate source, including mammals such as primates (e.g., humans) and rodents (e.g., mice and rats), unless otherwise indicated. The term encompasses “full-length,” unprocessed CD244 as well as any form of CD244 that results from processing in the cell. The term also encompasses naturally occurring variants of CD244, e.g., splice variants or allelic variants. The nucleic acid sequence of an exemplary human CD244 is shown under NCBI Reference Sequence: NM_016382.4. The amino acid sequence of an exemplary protein encoded by human CD244 is shown under UniProt Accession No. Q07763.

The term “CTSW” as used herein, refers to any native CTSW (Cathepsin W; also known as LYPN) from any vertebrate source, including mammals such as primates (e.g., humans) and rodents (e.g., mice and rats), unless otherwise indicated. The term encompasses “full-length,” unprocessed CTSW as well as any form of CTSW that results from processing in the cell. The term also encompasses naturally occurring variants of CTSW, e.g., splice variants or allelic variants. The nucleic acid sequence of an exemplary human CTSW is shown under NCBI Reference Sequence: NM_001335.4. The amino acid sequence of an exemplary protein encoded by human CTSW is shown under UniProt Accession No. P56202.

The term “FASLG” as used herein, refers to any native FASLG (Fas ligand; also known as FAS; CD95L; and CD178) from any vertebrate source, including mammals such as primates (e.g., humans) and rodents (e.g., mice and rats), unless otherwise indicated. The term encompasses “full-length,” unprocessed FASLG as well as any form of FASLG that results from processing in the cell. The term also encompasses naturally occurring variants of FASLG, e.g., splice variants or allelic variants. The nucleic acid sequence of an exemplary human FASLG is shown under NCBI Reference Sequence: NM_000639.3. The amino acid sequence of an exemplary protein encoded by human FASLG is shown under UniProt Accession No. P48023.

The term “GZMA” as used herein, refers to any native GZMA (Granzyme A; also known as CTLA3; and HFSP) from any vertebrate source, including mammals such as primates (e.g., humans) and rodents (e.g., mice and rats), unless otherwise indicated. The term encompasses “full-length,” unprocessed GZMA as well as any form of GZMA that results from processing in the cell. The term also encompasses naturally occurring variants of GZMA, e.g., splice variants or allelic variants. The nucleic acid sequence of an exemplary human GZMA is shown under NCBI Reference Sequence: NM_006144.4. The amino acid sequence of an exemplary protein encoded by human GZMA is shown under UniProt Accession No. P12544.

The term “GZMB” as used herein, refers to any native GZMB (Granzyme B; also known as CGL1 and CSPB) from any vertebrate source, including mammals such as primates (e.g., humans) and rodents (e.g., mice and rats), unless otherwise indicated. The term encompasses “full-length,” unprocessed GZMB as well as any form of GZMB that results from processing in the cell. The term also encompasses naturally occurring variants of GZMB, e.g., splice variants or allelic variants. The nucleic acid sequence of an exemplary human GZMB is shown under NCBI Reference Sequence: NM_004131.6. The amino acid sequence of an exemplary protein encoded by human GZMB is shown under UniProt Accession No. P10144.

The term “GZMH” as used herein, refers to any native GZMH (Granzyme H; also known as CGL2; CTSGL2; and CSPC) from any vertebrate source, including mammals such as primates (e.g., humans) and rodents (e.g., mice and rats), unless otherwise indicated. The term encompasses “full-length,” unprocessed GZMH as well as any form of GZMH that results from processing in the cell. The term also encompasses naturally occurring variants of GZMH, e.g., splice variants or allelic variants. The nucleic acid sequence of an exemplary human GZMH is shown under NCBI Reference Sequence: NM_033423.5. The amino acid sequence of an exemplary protein encoded by human GZMH is shown under UniProt Accession No. P20718.

The term “IL18RAP” as used herein, refers to any native IL18RAP (Interleukin 18 receptor accessory protein; also known as CDw218b) from any vertebrate source, including mammals such as primates (e.g., humans) and rodents (e.g., mice and rats), unless otherwise indicated. The term encompasses “full-length,” unprocessed IL18RAP as well as any form of IL18RAP that results from processing in the cell. The term also encompasses naturally occurring variants of IL18RAP, e.g., splice variants or allelic variants. The nucleic acid sequence of an exemplary human IL18RAP is shown under NCBI Reference Sequence: NM_003853.4. The amino acid sequence of an exemplary protein encoded by human IL18RAP is shown under UniProt Accession No. 095256.

The term “IL2RB” as used herein, refers to any native IL2RB (Interleukin-2 receptor subunit beta; also known as IL15RB; CD122; and P70-75) from any vertebrate source, including mammals such as primates (e.g., humans) and rodents (e.g., mice and rats), unless otherwise indicated. The term encompasses “full-length,” unprocessed IL2RB as well as any form of IL2RB that results from processing in the cell. The term also encompasses naturally occurring variants of IL2RB, e.g., splice variants or allelic variants. The nucleic acid sequence of an exemplary human IL2RB is shown under NCBI Reference Sequence: NM_000878.5. The amino acid sequence of an exemplary protein encoded by human IL2RB is shown under UniProt Accession No. P14784.

The term “KIR2DL4” as used herein, refers to any native KIR2DL4 (Killer cell immunoglobulin-like receptor 2DL4; also known as CD158D and KIR103AS) from any vertebrate source, including mammals such as primates (e.g., humans) and rodents (e.g., mice and rats), unless otherwise indicated. The term encompasses “full-length,” unprocessed KIR2DL4 as well as any form of KIR2DL4 that results from processing in the cell. The term also encompasses naturally occurring variants of KIR2DL4, e.g., splice variants or allelic variants. The nucleic acid sequence of an exemplary human KIR2DL4 is shown under NCBI Reference Sequence: NM_002255.6. The amino acid sequence of an exemplary protein encoded by human KIR2DL4 is shown under UniProt Accession No. Q99706.

The term “KLRB1” as used herein, refers to any native KLRB1 (Killer cell lectin-like receptor subfamily B, member 1; also known as NKR-P1A and CD161) from any vertebrate source, including mammals such as primates (e.g., humans) and rodents (e.g., mice and rats), unless otherwise indicated. The term encompasses “full-length,” unprocessed KLRB1 as well as any form of KLRB1 that results from processing in the cell. The term also encompasses naturally occurring variants of KLRB1, e.g., splice variants or allelic variants. The nucleic acid sequence of an exemplary human KLRB1 is shown under NCBI Reference Sequence: NM_002258.3. The amino acid sequence of an exemplary protein encoded by human KLRB1 is shown under UniProt Accession No. Q12918.

The term “KLRC3” as used herein, refers to any native KLRC3 (Killer cell lectin-like receptor C3; also known as NKG2E) from any vertebrate source, including mammals such as primates (e.g., humans) and rodents (e.g., mice and rats), unless otherwise indicated. The term encompasses “full-length,” unprocessed KLRC3 as well as any form of KLRC3 that results from processing in the cell. The term also encompasses naturally occurring variants of KLRC3, e.g., splice variants or allelic variants. The nucleic acid sequence of an exemplary human KLRC3 is shown under NCBI Reference Sequence: NM_002261.3. The amino acid sequence of an exemplary protein encoded by human KLRC3 is shown under UniProt Accession No. Q07444.

The term “KLRD1” as used herein, refers to any native KLRD1 (Killer cell lectin-like receptor D1; also known as CD94) from any vertebrate source, including mammals such as primates (e.g., humans) and rodents (e.g., mice and rats), unless otherwise indicated. The term encompasses “full-length,” unprocessed KLRD1 as well as any form of KLRD1 that results from processing in the cell. The term also encompasses naturally occurring variants of KLRD1, e.g., splice variants or allelic variants. The nucleic acid sequence of an exemplary human KLRD1 is shown under NCBI Reference Sequence: NM_002262.5. The amino acid sequence of an exemplary protein encoded by human KLRD1 is shown under UniProt Accession No. Q13241.

The term “KLRF1” as used herein, refers to any native KLRF1 (Killer cell lectin-like receptor subfamily F member 1; also known as CLEC5C) from any vertebrate source, including mammals such as primates (e.g., humans) and rodents (e.g., mice and rats), unless otherwise indicated. The term encompasses “full-length,” unprocessed KLRF1 as well as any form of KLRF1 that results from processing in the cell. The term also encompasses naturally occurring variants of KLRF1, e.g., splice variants or allelic variants. The nucleic acid sequence of an exemplary human KLRF1 is shown under NCBI Reference Sequence: NM_016523.3. The amino acid sequence of an exemplary protein encoded by human KLRF1 is shown under UniProt Accession No. Q9NZS2.

The term “KLRK1” as used herein, refers to any native KLRK1 (Killer cell lectin-like receptor subfamily K member 1; also known as NKG2D) from any vertebrate source, including mammals such as primates (e.g., humans) and rodents (e.g., mice and rats), unless otherwise indicated. The term encompasses “full-length,” unprocessed KLRK1 as well as any form of KLRK1 that results from processing in the cell. The term also encompasses naturally occurring variants of KLRK1, e.g., splice variants or allelic variants. The nucleic acid sequence of an exemplary human KLRK1 is shown under NCBI Reference Sequence: NM_007360.4. The amino acid sequence of an exemplary protein encoded by human KLRK1 is shown under UniProt Accession No. P26718.

The term “NCR1” as used herein, refers to any native NCR1 (Natural cytotoxicity triggering receptor 1; also known as CD335; NKP46; and LY94) from any vertebrate source, including mammals such as primates (e.g., humans) and rodents (e.g., mice and rats), unless otherwise indicated. The term encompasses “full-length,” unprocessed NCR1 as well as any form of NCR1 that results from processing in the cell. The term also encompasses naturally occurring variants of NCR1, e.g., splice variants or allelic variants. The nucleic acid sequence of an exemplary human NCR1 is shown under NCBI Reference Sequence: NM_004829.7. The amino acid sequence of an exemplary protein encoded by human NCR1 is shown under UniProt Accession No. 076036.

The term “NKG7” as used herein, refers to any native NKG7 (Natural killer cell granule protein 7; also known as GIG1; GMP-17; and p15-TIA-1) from any vertebrate source, including mammals such as primates (e.g., humans) and rodents (e.g., mice and rats), unless otherwise indicated. The term encompasses “full-length,” unprocessed NKG7 as well as any form of NKG7 that results from processing in the cell. The term also encompasses naturally occurring variants of NKG7, e.g., splice variants or allelic variants. The nucleic acid sequence of an exemplary human NKG7 is shown under NCBI Reference Sequence: NM_005601.4. The amino acid sequence of an exemplary protein encoded by human NKG7 is shown under UniProt Accession No. Q16617.

The term “PRF1” as used herein, refers to any native PRF1 (Perforin-1; also known as PFP) from any vertebrate source, including mammals such as primates (e.g., humans) and rodents (e.g., mice and rats), unless otherwise indicated. The term encompasses “full-length,” unprocessed PRF1 as well as any form of PRF1 that results from processing in the cell. The term also encompasses naturally occurring variants of PRF1, e.g., splice variants or allelic variants. The nucleic acid sequence of an exemplary human PRF1 is shown under NCBI Reference Sequence: NM_005041.6. The amino acid sequence of an exemplary protein encoded by human PRF1 is shown under UniProt Accession No. P14222.

The term “XCL1” as used herein, refers to any native XCL1 (Chemokine (C motif) ligand; also known as LTN and SCYC1) from any vertebrate source, including mammals such as primates (e.g., humans) and rodents (e.g., mice and rats), unless otherwise indicated. The term encompasses “full-length,” unprocessed XCL1 as well as any form of XCL1 that results from processing in the cell. The term also encompasses naturally occurring variants of XCL1, e.g., splice variants or allelic variants. The nucleic acid sequence of an exemplary human XCL1 is shown under NCBI Reference Sequence: NM_002995.3. The amino acid sequence of an exemplary protein encoded by human XCL1 is shown under UniProt Accession No. P47992.

The term “XCL2” as used herein, refers to any native XCL2 (Chemokine (C motif) ligand 2; also known as SCM1B and SCYC2) from any vertebrate source, including mammals such as primates (e.g., humans) and rodents (e.g., mice and rats), unless otherwise indicated. The term encompasses “full-length,” unprocessed XCL2 as well as any form of XCL2 that results from processing in the cell. The term also encompasses naturally occurring variants of XCL2, e.g., splice variants or allelic variants. The nucleic acid sequence of an exemplary human XCL2 is shown under NCBI Reference Sequence: NM_003175.4. The amino acid sequence of an exemplary protein encoded by human XCL2 is shown under UniProt Accession No. Q9UBD3.

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression,” “increased expression level,” “increased levels,” “elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., a housekeeping biomarker).

“Decreased expression,” “decreased expression level,” “decreased levels,” “reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., a housekeeping biomarker). In some embodiments, reduced expression is little or no expression.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

The term “diagnosis” is used herein to refer to the identification or classification of a molecular or pathological state, disease or condition (e.g., cancer (e.g., NSCLC)). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor (e.g., a liver tumor) or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+ T lymphocytes and/or CD4+ T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, the reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

For the purposes herein a “section” of a tissue sample is meant a single part or piece of a tissue sample, for example, a thin slice of tissue or cells cut from a tissue sample (e.g., a tumor sample). It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

The phrase “based on” when used herein means that the information about one or more biomarkers is used to inform a treatment decision, information provided on a package insert, or marketing/promotional guidance, and the like.

As used herein, the term “adverse event” or “AE” refers to any unfavorable and unintended sign (including an abnormal laboratory finding), symptom, or disease temporally associated with the use of a medical treatment or procedure that may or may not be considered related to the medical treatment or procedure. Adverse events may be classified by “grade,” as defined by the National Cancer Institute Common Terminology Criteria for Adverse Events v5.0 (NIH CTCAE). In some aspects, the AE is a low grade AE, e.g., a Grade 1 or Grade 2 AE. Grade 1 includes AEs that are asymptomatic or have mild symptoms. Grade 2 includes AEs that are moderate and limit age-appropriate instrumental activities of daily living (e.g., preparing meals, shopping for groceries or clothes) and that indicate local or noninvasive intervention. In other instances, the AE is a high grade AE, e.g., a Grade 3, Grade 4, or Grade 5 AE. Grade 3 includes AEs that are severe or medically significant, but not immediately life-threatening, and that indicate hospitalization or prolongation of hospitalization. Grade 4 includes AEs that have life-threatening consequences and indicate urgent intervention. Grade 5 includes AEs that result in or relate to death.

As used herein, the term “immune-mediated adverse event” or “imAE” refers to an adverse event or “adverse event of special interest” (“AESI”), as classified by the NIH CTCAE, that has a putative immune-related etiology. In some aspects, the imAE is an AESI occurring as a result of immune checkpoint inhibitor therapy. In some aspects, the imAE affects the respiratory tract, the endocrine system (“endocrine imAE”), the skin (“dermatological imAE” or “skin imAE”), or the gastrointestinal tract (“GI imAE”). In some aspects, the imAE is pneumonitis.

As used herein, the term “immune checkpoint inhibitor” refers to a therapeutic agent that targets at least one immune checkpoint protein to alter the regulation of an immune response, e.g., down-modulating, inhibiting, up-modulating, or activating an immune response. The term “immune checkpoint blockade” may be used to refer to a therapy comprising an immune checkpoint inhibitor. Immune checkpoint proteins are known in the art and include, without limitation, programmed cell death ligand 1 (PD-L1), TIGIT, cytotoxic T-lymphocyte antigen 4 (CTLA-4), programmed cell death 1 (PD-1), programmed cell death ligand 2 (PD-L2), V-domain Ig suppressor of T cell activation (VISTA), B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, LAG-3, BTLA, IDO, OX40, and A2aR. In some aspects, an immune checkpoint protein may be expressed on the surface of an activated T cell. Therapeutic agents that can act as immune checkpoint inhibitors useful in the methods of the present invention, include, but are not limited to, therapeutic agents that target one or more of PD-L1, TIGIT, PD-1, CTLA-4, PD-L2, VISTA, B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, LAG-3, BTLA, IDO, OX40, and A2aR. In some aspects, an immune checkpoint inhibitor enhances or suppresses the function of one or more targeted immune checkpoint proteins. In some aspects, the immune checkpoint inhibitor is a PD-1 axis binding antagonist, such as atezolizumab, as described herein.

### II. Therapeutic and Diagnostic Methods and Compositions for Lung Cancer

Provided herein are therapeutic and diagnostic methods for cancer (e.g., NSCLC) in which a patient may be identified for, selected for, and/or administered a treatment regimen comprising a PD-1 axis binding antagonist (e.g., atezolizumab) based on the patient's genotype comprising HLA or KIR genes or HLA/KIR pairs that are associated with improved NK cell education, e.g., at least one copy of HLA-C1, at least one copy of HLA-Bw4, at least one copy of KIR2DL3, and/or at least one copy of KIR3DL1. For example, in some examples, provided herein are therapeutic and diagnostic methods for cancer (e.g., NSCLC) in which a patient may be identified for, selected for, and/or administered a treatment regimen comprising a PD-1 axis binding antagonist (e.g., atezolizumab) based on the patient's genotype comprising at least one copy of HLA-C1 and/or at least one copy of KIR2DL3. In another example, provided herein are therapeutic and diagnostics methods for cancer (e.g., NSCLC) in which a patient may be identified for, selected for, and/or administered a treatment regimen comprising a PD-1 axis binding antagonist (e.g., atezolizumab) based on the patient's genotype comprising at least one copy of HLA-Bw4 and/or at least one copy of KIR3DL1. Without wishing to be bound by theory, such patients may have increased NK cell activity or function, e.g., due to improved NK cell education. Accordingly, such patients may also benefit from NK cell-directed therapy agents, either alone or in combination with a PD-1 axis binding antagonist (e.g., atezolizumab).

Also provided herein are therapeutic and diagnostic methods for cancer in which a patient may be identified for, selected for, and/or administered a treatment regimen comprising a PD-1 axis binding antagonist (e.g., atezolizumab) based on an increased level of NK cell infiltration in a tumor sample relative to a reference level of NK cell infiltration. Without wishing to be bound by theory, such patients may have increased NK cell activity or function, e.g., due to improved NK cell education. Accordingly, such patients may also benefit from NK cell-directed therapy agents, either alone or in combination with a PD-1 axis binding antagonist.

Also provided are in vitro methods for NK cell education, e.g., in which NK cells expressing KIR2DL3 or KIR3DL1 may be contacted with cells expressing HLA-C1 or HLA-Bw4, respectively. The resulting NK cells can be used, e.g., for adoptive cell therapy, e.g., for patients with HLA loss phenotypes.

In one example, provided herein is a method of treating a cancer (e.g., NSCLC) in a patient in need thereof whose genome has been determined to comprise at least one copy of HLA-C1, the method comprising administering to the patient an effective amount of a treatment regimen comprising a PD-1 axis binding antagonist (e.g., atezolizumab). In some instances, the patient's genome further comprises at least one copy of KIR2DL3.

In another example, provided herein is a PD-1 axis binding antagonist (e.g., atezolizumab) for use in treating a cancer (e.g., NSCLC) in a patient in need thereof whose genome has been determined to comprise at least one copy of HLA-C1. In some instances, the patient's genome further comprises at least one copy of KIR2DL3.

In another example, provided herein is a method of treating a cancer (e.g., NSCLC) in a patient in need thereof whose genome has been determined to comprise at least one copy of HLA-C1 and at least one copy of KIR2DL3, the method comprising administering to the patient an effective amount of a treatment regimen comprising a PD-1 axis binding antagonist (e.g., atezolizumab).

In another example, provided herein is a PD-1 axis binding antagonist (e.g., atezolizumab) for use in treating a cancer (e.g., NSCLC) in a patient in need thereof whose genome has been determined to comprise at least one copy of HLA-C1 and at least one copy of KIR2DL3.

In another example, provided herein is a method of treating a cancer (e.g., NSCLC) in a patient in need thereof whose genome has been determined to comprise at least one copy of HLA-Bw4, the method comprising administering to the patient an effective amount of a treatment regimen comprising a PD-1 axis binding antagonist (e.g., atezolizumab). In some instances, the patient's genome further comprises at least one copy of KIR3DL1.

In another example, provided herein is a PD-1 axis binding antagonist (e.g., atezolizumab) for use in treating a cancer (e.g., NSCLC) in a patient in need thereof whose genome has been determined to comprise at least one copy of HLA-Bw4. In some instances, the patient's genome further comprises at least one copy of KIR3DL1.

In another example, provided herein is a method of treating a cancer (e.g., NSCLC) in a patient in need thereof whose genome has been determined to comprise at least one copy of HLA-Bw4 and at least one copy of KIR3DL1, the method comprising administering to the patient an effective amount of a treatment regimen comprising a PD-1 axis binding antagonist (e.g., atezolizumab).

In another example, provided herein is a PD-1 axis binding antagonist (e.g., atezolizumab) for use in treating a cancer (e.g., NSCLC) in a patient in need thereof whose genome has been determined to comprise at least one copy of HLA-Bw4 and at least one copy of KIR3DL1.

In another example, provided herein is a method of treating a cancer (e.g., NSCLC) in a patient in need thereof, the method comprising: (a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-C1 in the patient's genome. In some instances, step (a) further comprises determining whether the patient's genome comprises at least one copy of KIR2DL3.

In another example, provided herein is a PD-1 axis binding antagonist (e.g., atezolizumab) for use in a method of treating a cancer (e.g., NSCLC) in a patient in need thereof, the method comprising: (a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-C1 in the patient's genome. In some instances, step (a) further comprises determining whether the patient's genome comprises at least one copy of KIR2DL3.

In another example, provided herein is a method of treating a cancer (e.g., NSCLC) in a patient in need thereof, the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-C1 and at least one copy of KIR2DL3 in the patient's genome.

In another example, provided herein is a PD-1 axis binding antagonist (e.g., atezolizumab) for use in treating a cancer (e.g., NSCLC) in a patient in need thereof, the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-C1 and at least one copy of KIR2DL3 in the patient's genome.

In another example, provided herein is a method of treating a cancer (e.g., NSCLC) in a patient in need thereof, the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-Bw4 in the patient's genome. In some instances, step (a) further comprises determining whether the patient's genome comprises at least one copy of KIR3DL1.

In another example, provided herein is a PD-1 axis binding antagonist (e.g., atezolizumab) for use in treating a cancer (e.g., NSCLC) in a patient in need thereof, the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-Bw4 in the patient's genome. In some instances, step (a) further comprises determining whether the patient's genome comprises at least one copy of KIR3DL1.

In another example, provided herein is a method of treating a cancer (e.g., NSCLC) in a patient in need thereof, the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-Bw4 and at least one copy of KIR3DL1 in the patient's genome.

In another example, provided herein is a PD-1 axis binding antagonist (e.g., atezolizumab) for use in treating a cancer (e.g., NSCLC) in a patient in need thereof, the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-Bw4 and at least one copy of KIR3DL1 in the patient's genome.

In another example, provided herein is a method of identifying a patient having a cancer (e.g., NSCLC) who may benefit from a treatment regimen comprising a PD-1 axis binding antagonist (e.g., atezolizumab), the method comprising determining whether the patient's genome comprises at least one copy of HLA-C1, wherein the presence of at least one copy of HLA-C1 in the patient's genome identifies the patient as one who may benefit from treatment with a treatment regimen comprising a PD-1 axis binding antagonist. In some instances, the method further comprises determining whether the patient's genome comprises at least one copy of KIR2DL3.

In another example, provided herein is a method of identifying a patient having a cancer (e.g., NSCLC) who may benefit from a treatment regimen comprising a PD-1 axis binding antagonist (e.g., atezolizumab), the method comprising: (a) performing germline whole genome sequencing (WGS) or whole exome sequencing (WES) by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-C1, wherein the presence of at least one copy of HLA-C1 in the patient's genome identifies the patient as one who may benefit from treatment with a treatment regimen comprising a PD-1 axis binding antagonist. In some instances, the method further comprises determining whether the patient's genome comprises at least one copy of KIR2DL3.

In another example, provided herein is a method of identifying a patient having a cancer (e.g., NSCLC) who may benefit from a treatment regimen comprising a PD-1 axis binding antagonist (e.g., atezolizumab), the method comprising determining whether the patient's genome comprises at least one copy of HLA-C1 and at least one copy of KIR2DL3, wherein the presence of at least one copy of HLA-C1 and at least one copy of KIR2DL3 in the patient's genome identifies the patient as one who may benefit from treatment with a treatment regimen comprising a PD-1 axis binding antagonist.

In another example, provided herein is a method of identifying a patient having a cancer (e.g., NSCLC) who may benefit from a treatment regimen comprising a PD-1 axis binding antagonist (e.g., atezolizumab), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-C1 and at least one copy of KIR2DL3, wherein the presence of at least one copy of HLA-C1 and at least one copy of KIR2DL3 in the patient's genome identifies the patient as one who may benefit from treatment with a treatment regimen comprising a PD-1 axis binding antagonist.

In another example, provided herein is a method of identifying a patient having a cancer (e.g., NSCLC) who may benefit from a treatment regimen comprising a PD-1 axis binding antagonist (e.g., atezolizumab), the method comprising determining whether the patient's genome comprises at least one copy of HLA-Bw4, wherein the presence of at least one copy of HLA-Bw4 in the patient's genome identifies the patient as one who may benefit from treatment with a treatment regimen comprising a PD-1 axis binding antagonist. In some instances, the method further comprises determining whether the patient's genome comprises at least one copy of KIR3DL1.

In another example, provided herein is a method of identifying a patient having a cancer (e.g., NSCLC) who may benefit from a treatment regimen comprising a PD-1 axis binding antagonist (e.g., atezolizumab), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-Bw4, wherein the presence of at least one copy of HLA-Bw4 in the patient's genome identifies the patient as one who may benefit from treatment with a treatment regimen comprising a PD-1 axis binding antagonist. In some instances, the method further comprises determining whether the patient's genome comprises at least one copy of KIR3DL1.

In another example, provided herein is a method of identifying a patient having a cancer (e.g., NSCLC) who may benefit from a treatment regimen comprising a PD-1 axis binding antagonist (e.g., atezolizumab), the method comprising determining whether the patient's genome comprises at least one copy of HLA-Bw4 and at least one copy of KIR3DL1, wherein the presence of at least one copy of HLA-Bw4 and at least one copy of KIR3DL1 in the patient's genome identifies the patient as one who may benefit from treatment with a treatment regimen comprising a PD-1 axis binding antagonist.

In another example, provided herein is a method of identifying a patient having a cancer (e.g., NSCLC) who may benefit from a treatment regimen comprising a PD-1 axis binding antagonist (e.g., atezolizumab), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-Bw4 and at least one copy of KIR3DL1, wherein the presence of at least one copy of HLA-Bw4 and at least one copy of KIR3DL1 in the patient's genome identifies the patient as one who may benefit from treatment with a treatment regimen comprising a PD-1 axis binding antagonist.

In another example, provided herein is a method for selecting a therapy for a patient having a cancer (e.g., NSCLC), the method comprising: (a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist (e.g., atezolizumab); and (b) selecting a treatment regimen comprising a PD-1 axis binding antagonist based on the presence of at least one copy of HLA-C1 in the patient's genome. In some instances, the method further comprises determining whether the patient's genome comprises at least one copy of KIR2DL3.

In another example, provided herein is a method for selecting a therapy for a patient having a cancer (e.g., NSCLC), the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist (e.g., atezolizumab); and (b) selecting a treatment regimen comprising a PD-1 axis binding antagonist based on the presence of at least one copy of HLA-C1 and at least one copy of KIR2DL3 in the patient's genome.

In another example, provided herein is a method for selecting a therapy for a patient having a cancer (e.g., NSCLC), the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist (e.g., atezolizumab); and (b) selecting a treatment regimen comprising a PD-1 axis binding antagonist based on the presence of at least one copy of HLA-Bw4 in the patient's genome. In some instances, the method further comprises determining whether the patient's genome comprises at least one copy of KIR3DL1.

In another example, provided herein is a method for selecting a therapy for a patient having a cancer (e.g., NSCLC), the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist (e.g., atezolizumab); and (b) selecting a treatment regimen comprising a PD-1 axis binding antagonist based on the presence of at least one copy of HLA-Bw4 and at least one copy of KIR3DL1 in the patient's genome.

Any of the preceding examples may further include administering an effective amount of a treatment regimen comprising a PD-1 axis binding antagonist (e.g., atezolizumab) to the patient.

In any of the examples described herein, the presence of HLA-C1, HLA-Bw4, KIR2DL3, and/or KIR3DL1 in the patient's genome may be determined using any suitable approach. For example, in some instances, the presence of HLA-C1, HLA-Bw4, KIR2DL3, and/or KIR3DL1 in the patient's genome is determined using next-generation sequencing, Sanger sequencing, a polymerase chain reaction (PCR)-based assay, or a single nucleotide polymorphism (SNP) array. In some instances, the next-generation sequencing comprises germline whole-genome sequencing or germline whole-exome sequencing. In some instances, the PCR-based assay comprises quantitative PCR (qPCR), typing using sequence-specific primers (SSP), or typing using sequence specific oligonucleotide probes (SSO).

In another example, provided herein is a method of treating a cancer (e.g., NSCLC) in a patient in need thereof who has been determined to have an increased level of natural killer (NK) cell infiltration in a tumor sample obtained from the patient relative to a reference level of NK cell infiltration, the method comprising administering to the patient an effective amount of a treatment regimen comprising a PD-1 axis binding antagonist (e.g., atezolizumab).

In another example, provided herein is a PD-1 axis binding antagonist for use in treating a cancer (e.g., NSCLC) in a patient in need thereof who has been determined to have an increased level of NK cell infiltration in a tumor sample obtained from the patient relative to a reference level of NK cell infiltration.

In another example, provided herein is a method of treating a cancer (e.g., NSCLC) in a patient in need thereof, the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the increased level of NK cell infiltration in the tumor sample obtained from the patient relative to the reference level of NK cell infiltration.

In another example, provided herein is a PD-1 axis binding antagonist (e.g., atezolizumab) for use in a method of treating a cancer (e.g., NSCLC) in a patient in need thereof, the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist; and (b) administering an effective amount of a treatment regimen comprising a PD-1 axis binding antagonist to the patient based on the increased level of NK cell infiltration in the tumor sample obtained from the patient relative to the reference level of NK cell infiltration.

In another example, provided herein is a method of identifying a patient having a cancer (e.g., NSCLC) who may benefit from a treatment regimen comprising a PD-1 axis binding antagonist (e.g., atezolizumab), the method comprising determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist.

In another example, provided herein is a method of identifying a patient having a cancer (e.g., NSCLC) who may benefit from a treatment regimen comprising a PD-1 axis binding antagonist (e.g., atezolizumab), the method comprising: (a) contacting a tumor sample obtained from the patient with one or more antibodies or nucleotide probes that bind to one or more NK cell markers to determine the level of NK cell infiltration in the tumor sample; and (b) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist. Any suitable antibodies or nucleotide probes may be used, e.g., antibodies or nucleotide probes that bind to any NK cell marker described herein or known in the art, e.g., one or more (e.g., 1, 2, 3, 4, 5, 6, 7, 8, 9, 10, 11, 12, 13, 14, 15, 16, 17, 18, 19, or all 20) of the following genes: CD160, CD244, CTSW, FASLG, GZMA, GZMB, GZMH, IL18RAP, IL2RB, KIR2DL4, KLRB1, KLRC3, KLRD1, KRLF1, KLRK1, NCR1, NKG7, PRF1, XCL1, and XCL2.

In another example, provided herein is a method for selecting a therapy for a patient having a cancer (e.g., NSCLC), the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist (e.g., atezolizumab); and (b) selecting a treatment regimen comprising a PD-1 axis binding antagonist based on the increased level of NK cell infiltration in the tumor sample obtained from the patient relative to the reference level of NK cell infiltration.

Any of the examples described herein may include administering an effective amount of a treatment regimen comprising a PD-1 axis binding antagonist (e.g., atezolizumab) to the patient.

In any of the examples described herein, the level of NK cell infiltration may be determined using any suitable approach. For example, in some instances, the level of NK cell infiltration is determined by determining an expression level of an NK cell gene signature, by counting a number of NK cells in the tumor sample, or by detecting the presence or level of one or more NK cell markers, e.g., by immunofluorescence, immunohistochemistry, western blot, flow cytometry, or any other suitable approach. Any suitable NK cell marker or combination of NK cell markers may be used. In some aspects, the NK cell marker is a costimulatory receptor, e.g., TRAIL, CD16a, CD16b, NKG2D, NKG2C, 4-11BB, OX40, CD27, 2B4, DNAM-1, NKp30, NKp46, NKp44, NKp80, KIR2DS1, and KIR2DS2. In some aspects, the NK cell receptor is a coinhibitory receptor. In some aspects, the coinhibitory receptor is a NKG2A or a KIR, e.g., KIR3DL1, KIR2DL1, KIR2DL2, or KIR2DL3.

In any of the examples described herein, the NK cell gene signature may include one or more (e.g., 1, 2, 3, 4, 5, 6, 7, 8, 9, 10, 11, 12, 13, 14, 15, 16, 17, 18, 19, or all 20) of the following genes: CD160, CD244, CTSW, FASLG, GZMA, GZMB, GZMH, IL18RAP, IL2RB, KIR2DL4, KLRB1, KLRC3, KLRD1, KRLF1, KLRK1, NCR1, NKG7, PRF1, XCL1, and XCL2. In some instances, the NK cell gene signature comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of the genes. In some instances, the reference level of NK cell infiltration is a median level. In some instances, the median level is a median level in a population of cancer (e.g., NSCLC) patients.

In some examples, the patient's genome comprises at least one copy (e.g., 1 or 2 copies) of HLA-C1 or at least one copy (e.g., 1 or 2 copies) of HLA-Bw4. In some instances, the patient's genome comprises at least one copy (e.g., 1 or 2 copies) of HLA-C1. In some instances, the patient's genome comprises at least one copy (e.g., 1 or 2 copies) of HLA-Bw4.

In some examples, the patient's genome comprises at least one copy (e.g., 1 or 2 copies) of KIR2DL3 or at least one copy (e.g., 1 or 2 copies) of KIR3DL1. In some instances, the patient's genome comprises at least one copy (e.g., 1 or 2 copies) of KIR2DL3. In some instances, the patient's genome comprises at least one copy (e.g., 1 or 2 copies) of KIR3DL1.

Any of the examples disclosed herein, including any of the preceding examples, may further include administering an effective amount of a treatment regimen comprising an NK cell-directed therapy agent (e.g., an NK cell engager) to the patient. Any suitable NK cell-directed therapy agent may be used, e.g., any NK cell-directed therapy agent described in Section V below. In some examples, any NK cell-directed therapy described in Hodgins et al. J. Clin. Invest. 129(9):3499-3510, 2019, may be used. In some instances, the NK cell-directed therapy agent comprises allogeneic NK cells, autologous NK cells, off-the-shelf NK cells, chimeric antigen receptor (CAR)-NK cells, cytokine therapy, an NK cell engager (e.g., a bispecific killer cell engager (BiKE), a tri-specific killer cell engager (TriKE), or a tetra-specific killer cell engager (TetraKE)), an NK cell checkpoint receptor antagonist, or an oncolytic virus.

In another example, provided herein is a method of treating a cancer (e.g., NSCLC) in a patient in need thereof whose genome has been determined to comprise at least one copy of HLA-C1, the method comprising administering to the patient an effective amount of a treatment regimen comprising an NK cell-directed therapy agent (e.g., an NK cell engager). In some instances, the patient's genome further comprises at least one copy of KIR2DL3.

In another example, provided herein is an NK cell-directed therapy agent (e.g., an NK cell engager) for use in treating a cancer (e.g., NSCLC) in a patient in need thereof whose genome has been determined to comprise at least one copy of HLA-C1. In some instances, the patient's genome further comprises at least one copy of KIR2DL3.

In another example, provided herein is a method of treating a cancer (e.g., NSCLC) in a patient in need thereof whose genome has been determined to comprise at least one copy of HLA-C1 and at least one copy of KIR2DL3, the method comprising administering to the patient an effective amount of a treatment regimen comprising an NK cell-directed therapy agent (e.g., an NK cell engager).

In another example, provided herein is an NK cell-directed therapy agent (e.g., an NK cell engager) for use in treating a cancer (e.g., NSCLC) in a patient in need thereof whose genome has been determined to comprise at least one copy of HLA-C1 and at least one copy of KIR2DL3.

In another example, provided herein is a method of treating a cancer (e.g., NSCLC) in a patient in need thereof whose genome has been determined to comprise at least one copy of HLA-Bw4, the method comprising administering to the patient an effective amount of a treatment regimen comprising an NK cell-directed therapy agent (e.g., an NK cell engager). In some instances, the patient's genome further comprises at least one copy of KIR3DL1.

In another example, provided herein is an NK cell-directed therapy agent (e.g., an NK cell engager) for use in treating a cancer (e.g., NSCLC) in a patient in need thereof whose genome has been determined to comprise at least one copy of HLA-Bw4. In some instances, the patient's genome further comprises at least one copy of KIR3DL1.

In another example, provided herein is a method of treating a cancer (e.g., NSCLC) in a patient in need thereof whose genome has been determined to comprise at least one copy of HLA-Bw4 and at least one copy of KIR3DL1, the method comprising administering to the patient an effective amount of a treatment regimen comprising an NK cell-directed therapy agent (e.g., an NK cell engager).

In another example, provided herein is an NK cell-directed therapy agent (e.g., an NK cell engager) for use in treating a cancer (e.g., NSCLC) in a patient in need thereof whose genome has been determined to comprise at least one copy of HLA-Bw4 and at least one copy of KIR3DL1.

In another example, provided herein is a method of treating a cancer (e.g., NSCLC) in a patient in need thereof, the method comprising: (a) determining whether the patient's genome comprises at least one copy of HLA-C1, wherein the presence of at least one copy of HLA-C1 in the patient's genome indicates that the patient is likely to benefit from a treatment regimen comprising an NK cell-directed therapy agent (e.g., an NK cell engager); and (b) administering an effective amount of a treatment regimen comprising an NK cell-directed therapy agent to the patient based on the presence of at least one copy of HLA-C1 in the patient's genome. In some instances, step (a) further comprises determining whether the patient's genome comprises at least one copy of KIR2DL3.

In another example, provided herein is an NK cell-directed therapy agent (e.g., an NK cell engager) for use in a method of treating a cancer (e.g., NSCLC) in a patient in need thereof, the method comprising: (a) determining whether the patient's genome comprises at least one copy of HLA-C1, wherein the presence of at least one copy of HLA-C1 in the patient's genome indicates that the patient is likely to benefit from a treatment regimen comprising an NK cell-directed therapy agent; and (b) administering an effective amount of a treatment regimen comprising an NK cell-directed therapy agent to the patient based on the presence of at least one copy of HLA-C1 in the patient's genome. In some instances, step (a) further comprises determining whether the patient's genome comprises at least one copy of KIR2DL3.

In another example, provided herein is a method of treating a cancer (e.g., NSCLC) in a patient in need thereof, the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising an NK cell-directed therapy agent (e.g., an NK cell engager); and (b) administering an effective amount of a treatment regimen comprising an NK cell-directed therapy agent to the patient based on the presence of at least one copy of HLA-C1 and at least one copy of KIR2DL3 in the patient's genome.

In another example, provided herein is an NK cell-directed therapy agent (e.g., an NK cell engager) for use in treating a cancer (e.g., NSCLC) in a patient in need thereof, the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising an NK cell-directed therapy agent; and (b) administering an effective amount of a treatment regimen comprising an NK cell-directed therapy agent to the patient based on the presence of at least one copy of HLA-C1 and at least one copy of KIR2DL3 in the patient's genome.

In another example, provided herein is a method of treating a cancer (e.g., NSCLC) in a patient in need thereof, the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising an NK cell-directed therapy agent (e.g., an NK cell engager); and (b) administering an effective amount of a treatment regimen comprising an NK cell-directed therapy agent to the patient based on the presence of at least one copy of HLA-Bw4 in the patient's genome. In some instances, step (a) further comprises determining whether the patient's genome comprises at least one copy of KIR3DL1.

In another example, provided herein is an NK cell-directed therapy agent (e.g., an NK cell engager) for use in treating a cancer (e.g., NSCLC) in a patient in need thereof, the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising an NK cell-directed therapy agent; and (b) administering an effective amount of a treatment regimen comprising an NK cell-directed therapy agent to the patient based on the presence of at least one copy of HLA-Bw4 in the patient's genome. In some instances, step (a) further comprises determining whether the patient's genome comprises at least one copy of KIR3DL1.

In another example, provided herein is a method of treating a cancer (e.g., NSCLC) in a patient in need thereof, the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising an NK cell-directed therapy agent (e.g., an NK cell engager); and (b) administering an effective amount of a treatment regimen comprising an NK cell-directed therapy agent to the patient based on the presence of at least one copy of HLA-Bw4 and at least one copy of KIR3DL1 in the patient's genome.

In another example, provided herein is an NK cell-directed therapy agent (e.g., an NK cell engager) for use in treating a cancer (e.g., NSCLC) in a patient in need thereof, the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising an NK cell-directed therapy agent; and (b) administering an effective amount of a treatment regimen comprising an NK cell-directed therapy agent to the patient based on the presence of at least one copy of HLA-Bw4 and at least one copy of KIR3DL1 in the patient's genome.

In another example, provided herein is a method of identifying a patient having a cancer (e.g., NSCLC) who may benefit from a treatment regimen comprising an NK cell-directed therapy agent (e.g., an NK cell engager), the method comprising determining whether the patient's genome comprises at least one copy of HLA-C1, wherein the presence of at least one copy of HLA-C1 in the patient's genome identifies the patient as one who may benefit from treatment with a treatment regimen comprising an NK cell-directed therapy agent. In some instances, the method further comprises determining whether the patient's genome comprises at least one copy of KIR2DL3.

In another example, provided herein is a method of identifying a patient having a cancer (e.g., NSCLC) who may benefit from a treatment regimen comprising an NK cell-directed therapy agent (e.g., an NK cell engager), the method comprising: (a) performing germline whole genome sequencing (WGS) or whole exome sequencing (WES) by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising an NK cell-directed therapy agent by determining whether the patient's genome comprises at least one copy of HLA-C1, wherein the presence of at least one copy of HLA-C1 in the patient's genome identifies the patient as one who may benefit from treatment with a treatment regimen comprising an NK cell-directed therapy agent. In some instances, the method further comprises determining whether the patient's genome comprises at least one copy of KIR2DL3.

In another example, provided herein is a method of identifying a patient having a cancer (e.g., NSCLC) who may benefit from a treatment regimen comprising an NK cell-directed therapy agent (e.g., an NK cell engager), the method comprising determining whether the patient's genome comprises at least one copy of HLA-C1 and at least one copy of KIR2DL3, wherein the presence of at least one copy of HLA-C1 and at least one copy of KIR2DL3 in the patient's genome identifies the patient as one who may benefit from treatment with a treatment regimen comprising an NK cell-directed therapy agent.

In another example, provided herein is a method of identifying a patient having a cancer (e.g., NSCLC) who may benefit from a treatment regimen comprising an NK cell-directed therapy agent (e.g., an NK cell engager), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising an NK cell-directed therapy agent by determining whether the patient's genome comprises at least one copy of HLA-C1 and at least one copy of KIR2DL3, wherein the presence of at least one copy of HLA-C1 and at least one copy of KIR2DL3 in the patient's genome identifies the patient as one who may benefit from treatment with a treatment regimen comprising an NK cell-directed therapy agent.

In another example, provided herein is a method of identifying a patient having a cancer (e.g., NSCLC) who may benefit from a treatment regimen comprising an NK cell-directed therapy agent (e.g., an NK cell engager), the method comprising determining whether the patient's genome comprises at least one copy of HLA-Bw4, wherein the presence of at least one copy of HLA-Bw4 in the patient's genome identifies the patient as one who may benefit from treatment with a treatment regimen comprising an NK cell-directed therapy agent. In some instances, the method further comprises determining whether the patient's genome comprises at least one copy of KIR3DL1.

In another example, provided herein is a method of identifying a patient having a cancer (e.g., NSCLC) who may benefit from a treatment regimen comprising an NK cell-directed therapy agent (e.g., an NK cell engager), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising an NK cell-directed therapy agent by determining whether the patient's genome comprises at least one copy of HLA-Bw4, wherein the presence of at least one copy of HLA-Bw4 in the patient's genome identifies the patient as one who may benefit from treatment with a treatment regimen comprising an NK cell-directed therapy agent. In some instances, the method further comprises determining whether the patient's genome comprises at least one copy of KIR3DL1.

In another example, provided herein is a method of identifying a patient having a cancer (e.g., NSCLC) who may benefit from a treatment regimen comprising an NK cell-directed therapy agent (e.g., an NK cell engager), the method comprising determining whether the patient's genome comprises at least one copy of HLA-Bw4 and at least one copy of KIR3DL1, wherein the presence of at least one copy of HLA-Bw4 and at least one copy of KIR3DL1 in the patient's genome identifies the patient as one who may benefit from treatment with a treatment regimen comprising an NK cell-directed therapy agent.

In another example, provided herein is a method of identifying a patient having a cancer (e.g., NSCLC) who may benefit from a treatment regimen comprising an NK cell-directed therapy agent (e.g., an NK cell engager), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising an NK cell-directed therapy agent by determining whether the patient's genome comprises at least one copy of HLA-Bw4 and at least one copy of KIR3DL1, wherein the presence of at least one copy of HLA-Bw4 and at least one copy of KIR3DL1 in the patient's genome identifies the patient as one who may benefit from treatment with a treatment regimen comprising an NK cell-directed therapy agent.

In another example, provided herein is a method for selecting a therapy for a patient having a cancer (e.g., NSCLC), the method comprising: (a) determining whether the patient's genome comprises at least one copy of HLA-C1, wherein the presence of at least one copy of HLA-C1 in the patient's genome indicates that the patient is likely to benefit from a treatment regimen comprising an NK cell-directed therapy agent (e.g., an NK cell engager); and (b) selecting a treatment regimen comprising an NK cell-directed therapy agent based on the presence of at least one copy of HLA-C1 in the patient's genome. In some instances, the method further comprises determining whether the patient's genome comprises at least one copy of KIR2DL3.

In another example, provided herein is a method for selecting a therapy for a patient having a cancer (e.g., NSCLC), the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising an NK cell-directed therapy agent (e.g., an NK cell engager); and (b) selecting a treatment regimen comprising an NK cell-directed therapy agent based on the presence of at least one copy of HLA-C1 and at least one copy of KIR2DL3 in the patient's genome.

In another example, provided herein is a method for selecting a therapy for a patient having a cancer (e.g., NSCLC), the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising an NK cell-directed therapy agent (e.g., an NK cell engager); and (b) selecting a treatment regimen comprising an NK cell-directed therapy agent based on the presence of at least one copy of HLA-Bw4 in the patient's genome. In some instances, the method further comprises determining whether the patient's genome comprises at least one copy of KIR3DL1.

In another example, provided herein is a method for selecting a therapy for a patient having a cancer (e.g., NSCLC), the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising an NK cell-directed therapy agent (e.g., an NK cell engager); and (b) selecting a treatment regimen comprising an NK cell-directed therapy agent based on the presence of at least one copy of HLA-Bw4 and at least one copy of KIR3DL1 in the patient's genome.

In any of the examples described herein, the presence of HLA-C1, HLA-Bw4, KIR2DL3, and/or KIR3DL1 in the patient's genome may be determined using any suitable approach. For example, in some instances, the presence of HLA-C1, HLA-Bw4, KIR2DL3, and/or KIR3DL1 in the patient's genome is determined using next-generation sequencing, Sanger sequencing, a polymerase chain reaction (PCR)-based assay, or a single nucleotide polymorphism (SNP) array. In some instances, the next-generation sequencing comprises germline whole-genome sequencing or germline whole-exome sequencing. In some instances, the PCR-based assay comprises quantitative PCR (qPCR), typing using sequence-specific primers (SSP), or typing using sequence specific oligonucleotide probes (SSO).

Any of the examples described herein may include administering an effective amount of a treatment regimen comprising an NK cell-directed therapy agent (e.g., an NK cell engager) to the patient.

In another example, provided herein is a method of treating a cancer (e.g., NSCLC) in a patient in need thereof who has been determined to have an increased level of natural killer (NK) cell infiltration in a tumor sample obtained from the patient relative to a reference level of NK cell infiltration, the method comprising administering to the patient an effective amount of a treatment regimen comprising an NK cell-directed therapy agent (e.g., an NK cell engager).

In another example, provided herein is an NK cell-directed therapy agent (e.g., an NK cell engager) for use in treating a cancer (e.g., NSCLC) in a patient in need thereof who has been determined to have an increased level of NK cell infiltration in a tumor sample obtained from the patient relative to a reference level of NK cell infiltration.

In another example, provided herein is a method of treating a cancer (e.g., NSCLC) in a patient in need thereof, the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising an NK cell-directed therapy agent (e.g., an NK cell engager); and (b) administering an effective amount of a treatment regimen comprising an NK cell-directed therapy agent to the patient based on the increased level of NK cell infiltration in the tumor sample obtained from the patient relative to the reference level of NK cell infiltration.

In another example, provided herein is an NK cell-directed therapy agent (e.g., an NK cell engager) for use in a method of treating a cancer (e.g., NSCLC) in a patient in need thereof, the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising an NK cell-directed therapy agent; and (b) administering an effective amount of a treatment regimen comprising an NK cell-directed therapy agent to the patient based on the increased level of NK cell infiltration in the tumor sample obtained from the patient relative to the reference level of NK cell infiltration.

In another example, provided herein is a method of identifying a patient having a cancer (e.g., NSCLC) who may benefit from a treatment regimen comprising an NK cell-directed therapy agent (e.g., an NK cell engager), the method comprising determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising an NK cell-directed therapy agent.

In another example, provided herein is a method of identifying a patient having a cancer (e.g., NSCLC) who may benefit from a treatment regimen comprising an NK cell-directed therapy agent (e.g., an NK cell engager), the method comprising: (a) contacting a tumor sample obtained from the patient with one or more antibodies or nucleotide probes that bind to one or more NK cell markers to determine the level of NK cell infiltration in the tumor sample; and (b) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising an NK cell-directed therapy agent.

Any suitable antibodies or nucleotide probes may be used, e.g., antibodies or nucleotide probes that bind to any NK cell marker described herein or known in the art, e.g., one or more (e.g., 1, 2, 3, 4, 5, 6, 7, 8, 9, 10, 11, 12, 13, 14, 15, 16, 17, 18, 19, or all 20) of the following genes: CD160, CD244, CTSW, FASLG, GZMA, GZMB, GZMH, IL18RAP, IL2RB, KIR2DL4, KLRB1, KLRC3, KLRD1, KRLF1, KLRK1, NCR1, NKG7, PRF1, XCL1, and XCL2.

In another example, provided herein is a method for selecting a therapy for a patient having a cancer (e.g., NSCLC), the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising an NK cell-directed therapy agent (e.g., an NK cell engager); and (b) selecting a treatment regimen comprising an NK cell-directed therapy agent based on the increased level of NK cell infiltration in the tumor sample obtained from the patient relative to the reference level of NK cell infiltration.

Any of the examples described herein may include administering an effective amount of a treatment regimen comprising an NK cell-directed therapy agent (e.g., an NK cell engager) to the patient.

In any of the examples described herein, the level of NK cell infiltration may be determined using any suitable approach. For example, in some instances, the level of NK cell infiltration is determined by determining an expression level of an NK cell gene signature, by counting a number of NK cells in the tumor sample, or by detecting the presence or level of one or more NK cell markers, e.g., by immunofluorescence, immunohistochemistry, western blot, flow cytometry, or any other suitable approach. Any suitable NK cell marker or combination of NK cell markers may be used. In some aspects, the NK cell marker is a costimulatory receptor, e.g., TRAIL, CD16a, CD16b, NKG2D, NKG2C, 4-11BB, OX40, CD27, 2B4, DNAM-1, NKp30, NKp46, NKp44, NKp80, KIR2DS1, and KIR2DS2. In some aspects, the NK cell receptor is a coinhibitory receptor. In some aspects, the coinhibitory receptor is a NKG2A or a KIR, e.g., KIR3DL1, KIR2DL1, KIR2DL2, or KIR2DL3.

In any of the examples described herein, the NK cell gene signature may include one or more (e.g., 1, 2, 3, 4, 5, 6, 7, 8, 9, 10, 11, 12, 13, 14, 15, 16, 17, 18, 19, or all 20) of the following genes: CD160, CD244, CTSW, FASLG, GZMA, GZMB, GZMH, IL18RAP, IL2RB, KIR2DL4, KLRB1, KLRC3, KLRD1, KRLF1, KLRK1, NCR1, NKG7, PRF1, XCL1, and XCL2. In some instances, the NK cell gene signature comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of the genes. In some instances, the reference level of NK cell infiltration is a median level. In some instances, the median level is a median level in a population of cancer (e.g., NSCLC) patients.

In some examples, the patient's genome comprises at least one copy (e.g., 1 or 2 copies) of HLA-C1 or at least one copy (e.g., 1 or 2 copies) of HLA-Bw4. In some instances, the patient's genome comprises at least one copy (e.g., 1 or 2 copies) of HLA-C1. In some instances, the patient's genome comprises at least one copy (e.g., 1 or 2 copies) of HLA-Bw4.

In some examples, the patient's genome comprises at least one copy (e.g., 1 or 2 copies) of KIR2DL3 or at least one copy (e.g., 1 or 2 copies) of KIR3DL1. In some instances, the patient's genome comprises at least one copy (e.g., 1 or 2 copies) of KIR2DL3. In some instances, the patient's genome comprises at least one copy (e.g., 1 or 2 copies) of KIR3DL1.

Any suitable NK cell-directed therapy agent may be used, e.g., any NK cell-directed therapy agent described in Section V below. Any suitable NK cell directed therapy may be used, including any NK cell-directed therapy described herein (see, e.g., Section V below). In some examples, any NK cell-directed therapy described in Hodgins et al. J. Clin. Invest. 129(9):3499-3510, 2019, may be used. In some instances, the NK cell-directed therapy agent comprises allogeneic NK cells, autologous NK cells, off-the-shelf NK cells, chimeric antigen receptor (CAR)-NK cells, cytokine therapy, an NK cell engager (e.g., a bispecific killer cell engager (BiKE), a tri-specific killer cell engager (TriKE), or a tetra-specific killer cell engager (TetraKE)), an NK cell checkpoint receptor antagonist, or an oncolytic virus.

Any of the preceding examples in which an NK cell-directed therapy agent is administered to the patient may further include administering a PD-1 axis binding antagonist (e.g., atezolizumab) to the patient.

Also provided herein are in vitro methods for NK cell education. For example, such methods may include contacting NK cells that express KIR2DL3 or KIR3DL1 with cells with express HLA-C1 or HLA-Bw4, e.g., under conditions and for a time sufficient for NK cell education. Such educated NK cells may be used for adoptive cell therapy, e.g., for patients with HLA loss phetypes.

For example, provided herein is an in vitro method of NK cell education comprising contacting an NK cell expressing KIR2DL3 with a cell that expresses HLA-C1, e.g., under conditions and for a time sufficient for NK cell education.

In another example, provided herein is an in vitro method of NK cell education comprising contacting an NK cella expressing KIR3DL1 with a cell that expresses HLA-Bw4, e.g., under conditions and for a time sufficient for NK cell education.

Such NK cells may express KIR2DL3 or KIR3DL1 endogenously, or may be engineered to express KIR2DL3 or KIR3DL1 (e.g., using gene editing or transduction (e.g., lentiviral transduction). Any suitable engineering approach may be used.

In some examples, NK cells educated in vitro as described herein may be used for adoptive cell therapy. For example, in some examples, NK cells educated in vitro as described herein may be used to treat patients (e.g., NSCLC patients) with HLA loss phenotypes. In some examples, NK cells educated in vitro as described herein may be used to treat a patient having a cancer (e.g., NSCLC) whose genome lacks HLA-C1, HLA-Bw4, KIR2DL3, and/or KIR3DL1. In some examples, the patient's genome lacks HLA-C1. In some examples, the patient's genome lacks HLA-Bw4. In some examples, the patient's genome lacks KIR2DL3. In some examples, the patient's genome lacsk KIR3DL1.

In another example, provided herein is a method of treating a cancer (e.g., NSCLC) in a patient in need thereof whose genome has been determined to lack KIR2DL3 or KIR3DL1, the method comprising administering to the patient an effective amount of a treatment regimen comprising an NK cell-directed therapy agent.

In another example, provided herein is a NK cell-directed therapy agent for use in treating a cancer (e.g., NSCLC) in a patient in need thereof whose genome has been determined to lack KIR2DL3 or KIR3DL1.

In another example, provided herein is a method of treating a cancer (e.g., NSCLC) in a patient in need thereof, the method comprising: (a) determining whether the patient's genome lacks KIR2DL3 or KIR3DL1, wherein the absence of KIR2DL3 or KIR3DL1 in the patient's genome indicates that the patient is likely to benefit from a treatment regimen comprising an NK cell-directed therapy agent; and (b) administering an effective amount of a treatment regimen comprising an NK cell-directed therapy agent to the patient based on the absence of KIR2DL3 or KIR3DL1 in the patient's genome.

In another example, provided herein is a NK cell-directed therapy agent for use in a method of treating a cancer (e.g., NSCLC) in a patient in need thereof, the method comprising: (a) determining whether the patient's genome lacks KIR2DL3 or KIR3DL1, wherein the absence of KIR2DL3 or KIR3DL1 in the patient's genome indicates that the patient is likely to benefit from a treatment regimen comprising an NK cell-directed therapy agent; and (b) administering an effective amount of a treatment regimen comprising an NK cell-directed therapy agent to the patient based on the absence of KIR2DL3 or KIR3DL1 in the patient's genome.

In another example, provided herein is a method of identifying a patient having a cancer (e.g., NSCLC) who may benefit from a treatment regimen comprising an NK cell-directed therapy agent, the method comprising determining whether the patient's genome lacks KIR2DL3 or KIR3DL1, wherein the absence of KIR2DL3 or KIR3DL1 in the patient's genome identifies the patient as one who may benefit from treatment with a treatment regimen comprising an NK cell-directed therapy agent.

In another example, provided herein is a method of identifying a patient having a cancer (e.g., NSCLC) who may benefit from a treatment regimen comprising an NK cell-directed therapy agent, the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising an NK cell-directed therapy agent by determining whether KIR2DL3 or KIR3DL1 are absent from the patient's genome, wherein the absence of KIR2DL3 or KIR3DL1 in the patient's genome identifies the patient as one who may benefit from treatment with a treatment regimen comprising an NK cell-directed therapy agent.

In another example, provided herein is a method for selecting a therapy for a patient having a cancer (e.g., NSCLC), the method comprising: (a) determining whether the patient's genome lacks KIR2DL3 or KIR3DL1, wherein the absence of KIR2DL3 or KIR3DL1 in the patient's genome identifies the patient as one who may benefit from treatment with a treatment regimen comprising an NK cell-directed therapy agent; and (b) selecting a treatment regimen comprising NK cell-directed therapy agent based on the absence of KIR2DL3 or KIR3DL1 in the patient's genome.

Any suitable NK cell directed therapy may be used, including any NK cell-directed therapy described herein (see, e.g., Section V below). In some examples, any NK cell-directed therapy described in Hodgins et al. J. Clin. Invest. 129(9):3499-3510, 2019, may be used. In some instances, the NK cell-directed therapy agent comprises allogeneic NK cells, autologous NK cells, off-the-shelf NK cells, chimeric antigen receptor (CAR)-NK cells, cytokine therapy, an NK cell engager (e.g., a bispecific killer cell engager (BiKE), a tri-specific killer cell engager (TriKE), or a tetra-specific killer cell engager (TetraKE)), an NK cell checkpoint receptor antagonist, or an oncolytic virus.

In some examples, the NK cell-directed therapy agent comprises allogeneic NK cells, autologous NK cells, off-the-shelf NK cells, or a combination thereof. In some instances, the NK cell-directed therapy agent comprises allogeneic NK cells. In other instances, the NK cell-directed therapy agent comprises autologous NK cells. In yet other instances, the NK cell-directed therapy agent comprises off-the-shelf NK cells.

In some examples, the allogeneic NK cells, the autologous NK cells, or the off-the-shelf NK cells are engineered to express KIR2DL3 or KIR3DL1. For example, in some instances, the allogeneic NK cells, the autologous NK cells, or the off-the-shelf NK cells are engineered to express KIR2DL3. For example, in other instances, the allogeneic NK cells, the autologous NK cells, or the off-the-shelf NK cells are engineered to express KIR3DL1.

In some examples, the patient's genome comprises at least one copy (e.g., 1 or 2 copies) of HLA-C1 or at least one copy (e.g., 1 or 2 copies) of HLA-Bw4. In some instances, the patient's genome comprises at least one copy (e.g., 1 or 2 copies) of HLA-C1. In some instances, the patient's genome comprises at least one copy (e.g., 1 or 2 copies) of HLA-Bw4.

In some examples, the patient's genome comprises at least one copy (e.g., 1 or 2 copies) of KIR2DL3 or at least one copy (e.g., 1 or 2 copies) of KIR3DL1. In some instances, the patient's genome comprises at least one copy (e.g., 1 or 2 copies) of KIR2DL3. In some instances, the patient's genome comprises at least one copy (e.g., 1 or 2 copies) of KIR3DL1.

In some examples, treatment with the allogeneic NK cells, the autologous NK cells, or the off-the-shelf NK cells engineered to express KIR2DL3 or KIR3DL1 results in the patient being one who may benefit from treatment with a treatment regimen comprising a PD-1 axis binding antagonist (e.g., atezolizumab).

Any of the preceding examples may further include administering a treatment regimen comprising a PD-1 axis binding antagonist (e.g., atezolizumab) to the patient, e.g., before, concurrently, or after treatment with a treatment regimen comprising an NK cell-directed therapy agent.

In some examples, the benefit is in terms of improved overall survival (OS) or improved progression-free survival (PFS). In some instances, the benefit is in terms of improved OS. In some instances, the benefit is in terms of improved PFS. In some instances, improvement is relative to treatment with a treatment regimen that does not comprise the PD-1 axis binding antagonist (e.g., atezolizumab).

The cancer may be any suitable cancer. For example, in some examples, the cancer is a lung cancer (e.g., NSCLC), a renal cancer (e.g., renal cell carcinoma), or melanoma.

In some examples, the cancer is NSCLC. In some instances, the NSCLC is non-squamous NSCLC or squamous NSCLC. In some instances, the NSCLC is non-squamous NSCLC. In some instances, the non-squamous NSCLC is locally advanced or metastatic non-squamous NSCLC. In some instances, the non-squamous NSCLC is metastatic non-squamous NSCLC. In other instances, the NSCLC is squamous NSCLC. In some instances, the squamous NSCLC is locally advanced or metastatic squamous NSCLC. In some instances, the squamous NSCLC is metastatic squamous NSCLC.

For example, in one example, provided herein is a method of treating NSCLC in a patient in need thereof whose genome has been determined to comprise at least one copy of HLA-C1, the method comprising administering to the patient an effective amount of a treatment regimen comprising a PD-1 axis binding antagonist (e.g., atezolizumab). In some instances, the patient's genome further comprises at least one copy of KIR2DL3.

In another example, provided herein is a PD-1 axis binding antagonist (e.g., atezolizumab) for use in treating NSCLC in a patient in need thereof whose genome has been determined to comprise at least one copy of HLA-C1. In some instances, the patient's genome further comprises at least one copy of KIR2DL3.

In another example, provided herein is a method of treating NSCLC in a patient in need thereof whose genome has been determined to comprise at least one copy of HLA-C1 and at least one copy of KIR2DL3, the method comprising administering to the patient an effective amount of a treatment regimen comprising a PD-1 axis binding antagonist (e.g., atezolizumab).

In another example, provided herein is a PD-1 axis binding antagonist (e.g., atezolizumab) for use in treating NSCLC in a patient in need thereof whose genome has been determined to comprise at least one copy of HLA-C1 and at least one copy of KIR2DL3.

In another example, provided herein is a method of treating NSCLC in a patient in need thereof whose genome has been determined to comprise at least one copy of HLA-Bw4, the method comprising administering to the patient an effective amount of a treatment regimen comprising a PD-1 axis binding antagonist (e.g., atezolizumab). In some instances, the patient's genome further comprises at least one copy of KIR3DL1.

In another example, provided herein is a PD-1 axis binding antagonist (e.g., atezolizumab) for use in treating NSCLC in a patient in need thereof whose genome has been determined to comprise at least one copy of HLA-Bw4. In some instances, the patient's genome further comprises at least one copy of KIR3DL1.

In another example, provided herein is a method of treating NSCLC in a patient in need thereof whose genome has been determined to comprise at least one copy of HLA-Bw4 and at least one copy of KIR3DL1, the method comprising administering to the patient an effective amount of a treatment regimen comprising a PD-1 axis binding antagonist (e.g., atezolizumab).

In another example, provided herein is a PD-1 axis binding antagonist (e.g., atezolizumab) for use in treating NSCLC in a patient in need thereof whose genome has been determined to comprise at least one copy of HLA-Bw4 and at least one copy of KIR3DL1.

In another example, provided herein is a method of treating NSCLC in a patient in need thereof, the method comprising: (a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-C1 in the patient's genome. In some instances, step (a) further comprises determining whether the patient's genome comprises at least one copy of KIR2DL3.

In another example, provided herein is a PD-1 axis binding antagonist (e.g., atezolizumab) for use in a method of treating NSCLC in a patient in need thereof, the method comprising: (a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-C1 in the patient's genome. In some instances, step (a) further comprises determining whether the patient's genome comprises at least one copy of KIR2DL3.

In another example, provided herein is a method of treating NSCLC in a patient in need thereof, the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-C1 and at least one copy of KIR2DL3 in the patient's genome.

In another example, provided herein is a PD-1 axis binding antagonist (e.g., atezolizumab) for use in treating NSCLC in a patient in need thereof, the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-C1 and at least one copy of KIR2DL3 in the patient's genome.

In another example, provided herein is a method of treating NSCLC in a patient in need thereof, the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-Bw4 in the patient's genome. In some instances, step (a) further comprises determining whether the patient's genome comprises at least one copy of KIR3DL1.

In another example, provided herein is a PD-1 axis binding antagonist (e.g., atezolizumab) for use in treating NSCLC in a patient in need thereof, the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-Bw4 in the patient's genome. In some instances, step (a) further comprises determining whether the patient's genome comprises at least one copy of KIR3DL1.

In another example, provided herein is a method of treating NSCLC in a patient in need thereof, the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-Bw4 and at least one copy of KIR3DL1 in the patient's genome.

In another example, provided herein is a PD-1 axis binding antagonist for use in treating NSCLC in a patient in need thereof, the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-Bw4 and at least one copy of KIR3DL1 in the patient's genome.

In another example, provided herein is a method of identifying a patient having NSCLC who may benefit from a treatment regimen comprising a PD-1 axis binding antagonist (e.g., atezolizumab), the method comprising determining whether the patient's genome comprises at least one copy of HLA-C1, wherein the presence of at least one copy of HLA-C1 in the patient's genome identifies the patient as one who may benefit from treatment with a treatment regimen comprising a PD-1 axis binding antagonist. In some instances, the method further comprises determining whether the patient's genome comprises at least one copy of KIR2DL3.

In another example, provided herein is a method of identifying a patient having NSCLC who may benefit from a treatment regimen comprising a PD-1 axis binding antagonist (e.g., atezolizumab), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-C1, wherein the presence of at least one copy of HLA-C1 in the patient's genome identifies the patient as one who may benefit from treatment with a treatment regimen comprising a PD-1 axis binding antagonist. In some instances, the method further comprises determining whether the patient's genome comprises at least one copy of KIR2DL3.

In another example, provided herein is a method of identifying a patient having NSCLC who may benefit from a treatment regimen comprising a PD-1 axis binding antagonist (e.g., atezolizumab), the method comprising determining whether the patient's genome comprises at least one copy of HLA-C1 and at least one copy of KIR2DL3, wherein the presence of at least one copy of HLA-C1 and at least one copy of KIR2DL3 in the patient's genome identifies the patient as one who may benefit from treatment with a treatment regimen comprising a PD-1 axis binding antagonist.

In another example, provided herein is a method of identifying a patient having NSCLC who may benefit from a treatment regimen comprising a PD-1 axis binding antagonist (e.g., atezolizumab), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-C1 and at least one copy of KIR2DL3, wherein the presence of at least one copy of HLA-C1 and at least one copy of KIR2DL3 in the patient's genome identifies the patient as one who may benefit from treatment with a treatment regimen comprising a PD-1 axis binding antagonist.

In another example, provided herein is a method of identifying a patient having NSCLC who may benefit from a treatment regimen comprising a PD-1 axis binding antagonist (e.g., atezolizumab), the method comprising determining whether the patient's genome comprises at least one copy of HLA-Bw4, wherein the presence of at least one copy of HLA-Bw4 in the patient's genome identifies the patient as one who may benefit from treatment with a treatment regimen comprising a PD-1 axis binding antagonist. In some instances, the method further comprises determining whether the patient's genome comprises at least one copy of KIR3DL1.

In another example, provided herein is a method of identifying a patient having NSCLC who may benefit from a treatment regimen comprising a PD-1 axis binding antagonist (e.g., atezolizumab), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-Bw4, wherein the presence of at least one copy of HLA-Bw4 in the patient's genome identifies the patient as one who may benefit from treatment with a treatment regimen comprising a PD-1 axis binding antagonist. In some instances, the method further comprises determining whether the patient's genome comprises at least one copy of KIR3DL1.

In another example, provided herein is a method of identifying a patient having NSCLC who may benefit from a treatment regimen comprising a PD-1 axis binding antagonist (e.g., atezolizumab), the method comprising determining whether the patient's genome comprises at least one copy of HLA-Bw4 and at least one copy of KIR3DL1, wherein the presence of at least one copy of HLA-Bw4 and at least one copy of KIR3DL1 in the patient's genome identifies the patient as one who may benefit from treatment with a treatment regimen comprising a PD-1 axis binding antagonist.

In another example, provided herein is a method of identifying a patient having NSCLC who may benefit from a treatment regimen comprising a PD-1 axis binding antagonist (e.g., atezolizumab), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-Bw4 and at least one copy of KIR3DL1, wherein the presence of at least one copy of HLA-Bw4 and at least one copy of KIR3DL1 in the patient's genome identifies the patient as one who may benefit from treatment with a treatment regimen comprising a PD-1 axis binding antagonist.

In another example, provided herein is a method for selecting a therapy for a patient having NSCLC, the method comprising: (a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist (e.g., atezolizumab); and (b) selecting a treatment regimen comprising a PD-1 axis binding antagonist based on the presence of at least one copy of HLA-C1 in the patient's genome. In some instances, the method further comprises determining whether the patient's genome comprises at least one copy of KIR2DL3.

In another example, provided herein is a method for selecting a therapy for a patient having NSCLC, the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist (e.g., atezolizumab); and (b) selecting a treatment regimen comprising a PD-1 axis binding antagonist based on the presence of at least one copy of HLA-C1 and at least one copy of KIR2DL3 in the patient's genome.

In another example, provided herein is a method for selecting a therapy for a patient having NSCLC, the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist (e.g., atezolizumab); and (b) selecting a treatment regimen comprising a PD-1 axis binding antagonist based on the presence of at least one copy of HLA-Bw4 in the patient's genome. In some instances, the method further comprises determining whether the patient's genome comprises at least one copy of KIR3DL1.

In another example, provided herein is a method for selecting a therapy for a patient having NSCLC, the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist (e.g., atezolizumab); and (b) selecting a treatment regimen comprising a PD-1 axis binding antagonist based on the presence of at least one copy of HLA-Bw4 and at least one copy of KIR3DL1 in the patient's genome.

Any of the preceding examples may further include administering an effective amount of a treatment regimen comprising a PD-1 axis binding antagonist to the patient.

In any of the examples described herein, the presence of HLA-C1, HLA-Bw4, KIR2DL3, and/or KIR3DL1 in the patient's genome may be determined using any suitable approach. For example, in some instances, the presence of HLA-C1, HLA-Bw4, KIR2DL3, and/or KIR3DL1 in the patient's genome is determined using next-generation sequencing, Sanger sequencing, a polymerase chain reaction (PCR)-based assay, or a single nucleotide polymorphism (SNP) array. In some instances, the next-generation sequencing comprises germline whole-genome sequencing or germline whole-exome sequencing. In some instances, the PCR-based assay comprises quantitative PCR (qPCR), typing using sequence-specific primers (SSP), or typing using sequence specific oligonucleotide probes (SSO).

In another example, provided herein is a method of treating NSCLC in a patient in need thereof who has been determined to have an increased level of NK cell infiltration in a tumor sample obtained from the patient relative to a reference level of NK cell infiltration, the method comprising administering to the patient an effective amount of a treatment regimen comprising a PD-1 axis binding antagonist (e.g., atezolizumab).

In another example, provided herein is a PD-1 axis binding antagonist (e.g., atezolizumab) for use in treating NSCLC in a patient in need thereof who has been determined to have an increased level of NK cell infiltration in a tumor sample obtained from the patient relative to a reference level of NK cell infiltration.

In another example, provided herein is a method of treating NSCLC in a patient in need thereof, the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the increased level of NK cell infiltration in the tumor sample obtained from the patient relative to the reference level of NK cell infiltration.

In another example, provided herein is a PD-1 axis binding antagonist (e.g., atezolizumab) for use in a method of treating NSCLC in a patient in need thereof, the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist; and (b) administering an effective amount of a treatment regimen comprising a PD-1 axis binding antagonist to the patient based on the increased level of NK cell infiltration in the tumor sample obtained from the patient relative to the reference level of NK cell infiltration.

In another example, provided herein is a method of identifying a patient having NSCLC who may benefit from a treatment regimen comprising a PD-1 axis binding antagonist (e.g., atezolizumab), the method comprising determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist.

In another example, provided herein is a method of identifying a patient having NSCLC who may benefit from a treatment regimen comprising a PD-1 axis binding antagonist (e.g., atezolizumab), the method comprising: (a) contacting a tumor sample obtained from the patient with one or more antibodies or nucleotide probes that bind to one or more NK cell markers to determine the level of NK cell infiltration in the tumor sample; and (b) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist.

In another example, provided herein is a method for selecting a therapy for a patient having NSCLC, the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist (e.g., atezolizumab); and (b) selecting a treatment regimen comprising a PD-1 axis binding antagonist based on the increased level of NK cell infiltration in the tumor sample obtained from the patient relative to the reference level of NK cell infiltration.

Any of the examples described herein may include administering an effective amount of a treatment regimen comprising a PD-1 axis binding antagonist (e.g., atezolizumab) to the patient.

In any of the examples described herein, the level of NK cell infiltration may be determined using any suitable approach. For example, in some instances, the level of NK cell infiltration is determined by determining an expression level of an NK cell gene signature, by counting a number of NK cells in the tumor sample, or by detecting the presence or level of one or more NK cell markers, e.g., by immunofluorescence, immunohistochemistry, western blot, flow cytometry, or any other suitable approach.

In any of the examples described herein, the NK cell gene signature may include one or more (e.g., 1, 2, 3, 4, 5, 6, 7, 8, 9, 10, 11, 12, 13, 14, 15, 16, 17, 18, 19, or all 20) of the following genes: CD160, CD244, CTSW, FASLG, GZMA, GZMB, GZMH, IL18RAP, IL2RB, KIR2DL4, KLRB1, KLRC3, KLRD1, KRLF1, KLRK1, NCR1, NKG7, PRF1, XCL1, and XCL2. In some instances, the NK cell gene signature comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of the genes. In some instances, the reference level of NK cell infiltration is a median level. In some instances, the median level is a median level in a population of NSCLC patients.

In another example, provided herein is a method of treating NSCLC in a patient in need thereof whose genome has been determined to lack KIR2DL3 or KIR3DL1, the method comprising administering to the patient an effective amount of a treatment regimen comprising an NK cell-directed therapy agent.

In another example, provided herein is a NK cell-directed therapy agent for use in treating NSCLC in a patient in need thereof whose genome has been determined to lack KIR2DL3 or KIR3DL1.

In another example, provided herein is a method of treating NSCLC in a patient in need thereof, the method comprising: (a) determining whether the patient's genome lacks KIR2DL3 or KIR3DL1, wherein the absence of KIR2DL3 or KIR3DL1 in the patient's genome indicates that the patient is likely to benefit from a treatment regimen comprising an NK cell-directed therapy agent; and (b) administering an effective amount of a treatment regimen comprising an NK cell-directed therapy agent to the patient based on the absence of KIR2DL3 or KIR3DL1 in the patient's genome.

In another example, provided herein is a NK cell-directed therapy agent for use in a method of treating NSCLC in a patient in need thereof, the method comprising: (a) determining whether the patient's genome lacks KIR2DL3 or KIR3DL1, wherein the absence of KIR2DL3 or KIR3DL1 in the patient's genome indicates that the patient is likely to benefit from a treatment regimen comprising an NK cell-directed therapy agent; and (b) administering an effective amount of a treatment regimen comprising an NK cell-directed therapy agent to the patient based on the absence of KIR2DL3 or KIR3DL1 in the patient's genome.

In another example, provided herein is a method of identifying a patient having NSCLC who may benefit from a treatment regimen comprising an NK cell-directed therapy agent, the method comprising determining whether the patient's genome lacks KIR2DL3 or KIR3DL1, wherein the absence of KIR2DL3 or KIR3DL1 in the patient's genome identifies the patient as one who may benefit from treatment with a treatment regimen comprising an NK cell-directed therapy agent.

In another example, provided herein is a method of identifying a patient having NSCLC who may benefit from a treatment regimen comprising an NK cell-directed therapy agent, the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising an NK cell-directed therapy agent by determining whether KIR2DL3 or KIR3DL1 are absent from the patient's genome, wherein the absence of KIR2DL3 or KIR3DL1 in the patient's genome identifies the patient as one who may benefit from treatment with a treatment regimen comprising an NK cell-directed therapy agent.

In another example, provided herein is a method for selecting a therapy for a patient having a cancer (e.g., NSCLC), the method comprising: (a) determining whether the patient's genome lacks KIR2DL3 or KIR3DL1, wherein the absence of KIR2DL3 or KIR3DL1 in the patient's genome identifies the patient as one who may benefit from treatment with a treatment regimen comprising an NK cell-directed therapy agent; and (b) selecting a treatment regimen comprising NK cell-directed therapy agent based on the absence of KIR2DL3 or KIR3DL1 in the patient's genome.

Any suitable NK cell directed therapy may be used, including any NK cell-directed therapy described herein (see, e.g., Section V below). In some examples, any NK cell-directed therapy described in Hodgins et al. J. Clin. Invest. 129(9):3499-3510, 2019, may be used. In some instances, the NK cell-directed therapy agent comprises allogeneic NK cells, autologous NK cells, off-the-shelf NK cells, chimeric antigen receptor (CAR)-NK cells, cytokine therapy, an NK cell engager (e.g., a bispecific killer cell engager (BiKE), a tri-specific killer cell engager (TriKE), or a tetra-specific killer cell engager (TetraKE)), an NK cell checkpoint receptor antagonist, or an oncolytic virus.

In some examples, the NK cell-directed therapy agent comprises allogeneic NK cells, autologous NK cells, off-the-shelf NK cells, or a combination thereof. In some instances, the NK cell-directed therapy agent comprises allogeneic NK cells. In other instances, the NK cell-directed therapy agent comprises autologous NK cells. In yet other instances, the NK cell-directed therapy agent comprises off-the-shelf NK cells.

In some examples, the allogeneic NK cells, the autologous NK cells, or the off-the-shelf NK cells are engineered to express KIR2DL3 or KIR3DL1. For example, in some instances, the allogeneic NK cells, the autologous NK cells, or the off-the-shelf NK cells are engineered to express KIR2DL3. For example, in other instances, the allogeneic NK cells, the autologous NK cells, or the off-the-shelf NK cells are engineered to express KIR3DL1.

In some examples, the patient's genome comprises at least one copy (e.g., 1 or 2 copies) of HLA-C1 or at least one copy (e.g., 1 or 2 copies) of HLA-Bw4. In some instances, the patient's genome comprises at least one copy (e.g., 1 or 2 copies) of HLA-C1. In some instances, the patient's genome comprises at least one copy (e.g., 1 or 2 copies) of HLA-Bw4.

In some examples, treatment with the allogeneic NK cells, the autologous NK cells, or the off-the-shelf NK cells engineered to express KIR2DL3 or KIR3DL1 results in the patient being one who may benefit from treatment with a treatment regimen comprising a PD-1 axis binding antagonist (e.g., atezolizumab).

Any of the preceding examples may further include administering a treatment regimen comprising a PD-1 axis binding antagonist (e.g., atezolizumab) to the patient, e.g., before, concurrently, or after treatment with a treatment regimen comprising an NK cell-directed therapy agent.

In some examples, the benefit is in terms of improved overall survival (OS) or improved progression-free survival (PFS). In some instances, the benefit is in terms of improved OS. In some instances, the benefit is in terms of improved PFS. In some instances, improvement is relative to treatment with a treatment regimen that does not comprise the PD-1 axis binding antagonist (e.g., atezolizumab).

In some examples, the cancer is a renal cancer. In some instances, the renal cancer is RCC. In some instances, the RCC is locally advanced or metastatic RCC.

For example, provided herein is a method of treating a renal caner (e.g., RCC) in a patient in need thereof whose genome has been determined to comprise at least one copy of HLA-C1, the method comprising administering to the patient an effective amount of a treatment regimen comprising a PD-1 axis binding antagonist (e.g., atezolizumab). In some instances, the patient's genome further comprises at least one copy of KIR2DL3.

In another example, provided herein is a PD-1 axis binding antagonist (e.g., atezolizumab) for use in treating a renal caner (e.g., RCC) in a patient in need thereof whose genome has been determined to comprise at least one copy of HLA-C1. In some instances, the patient's genome further comprises at least one copy of KIR2DL3.

In another example, provided herein is a method of treating a renal caner (e.g., RCC) in a patient in need thereof whose genome has been determined to comprise at least one copy of HLA-C1 and at least one copy of KIR2DL3, the method comprising administering to the patient an effective amount of a treatment regimen comprising a PD-1 axis binding antagonist (e.g., atezolizumab).

In another example, provided herein is a PD-1 axis binding antagonist (e.g., atezolizumab) for use in treating a renal caner (e.g., RCC) in a patient in need thereof whose genome has been determined to comprise at least one copy of HLA-C1 and at least one copy of KIR2DL3.

In another example, provided herein is a method of treating a renal caner (e.g., RCC) in a patient in need thereof whose genome has been determined to comprise at least one copy of HLA-Bw4, the method comprising administering to the patient an effective amount of a treatment regimen comprising a PD-1 axis binding antagonist (e.g., atezolizumab). In some instances, the patient's genome further comprises at least one copy of KIR3DL1.

In another example, provided herein is a PD-1 axis binding antagonist (e.g., atezolizumab) for use in treating a renal caner (e.g., RCC) in a patient in need thereof whose genome has been determined to comprise at least one copy of HLA-Bw4. In some instances, the patient's genome further comprises at least one copy of KIR3DL1.

In another example, provided herein is a method of treating a renal caner (e.g., RCC) in a patient in need thereof whose genome has been determined to comprise at least one copy of HLA-Bw4 and at least one copy of KIR3DL1, the method comprising administering to the patient an effective amount of a treatment regimen comprising a PD-1 axis binding antagonist (e.g., atezolizumab).

In another example, provided herein is a PD-1 axis binding antagonist (e.g., atezolizumab) for use in treating a renal caner (e.g., RCC) in a patient in need thereof whose genome has been determined to comprise at least one copy of HLA-Bw4 and at least one copy of KIR3DL1.

In another example, provided herein is a method of treating a renal caner (e.g., RCC) in a patient in need thereof, the method comprising: (a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-C1 in the patient's genome. In some instances, step (a) further comprises determining whether the patient's genome comprises at least one copy of KIR2DL3.

In another example, provided herein is a PD-1 axis binding antagonist (e.g., atezolizumab) for use in a method of treating a renal caner (e.g., RCC) in a patient in need thereof, the method comprising: (a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-C1 in the patient's genome. In some instances, step (a) further comprises determining whether the patient's genome comprises at least one copy of KIR2DL3.

In another example, provided herein is a method of treating a renal caner (e.g., RCC) in a patient in need thereof, the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-C1 and at least one copy of KIR2DL3 in the patient's genome.

In another example, provided herein is a PD-1 axis binding antagonist (e.g., atezolizumab) for use in treating a renal caner (e.g., RCC) in a patient in need thereof, the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-C1 and at least one copy of KIR2DL3 in the patient's genome.

In another example, provided herein is a method of treating a renal caner (e.g., RCC) in a patient in need thereof, the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-Bw4 in the patient's genome. In some instances, step (a) further comprises determining whether the patient's genome comprises at least one copy of KIR3DL1.

In another example, provided herein is a PD-1 axis binding antagonist (e.g., atezolizumab) for use in treating a renal caner (e.g., RCC) in a patient in need thereof, the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-Bw4 in the patient's genome. In some instances, step (a) further comprises determining whether the patient's genome comprises at least one copy of KIR3DL1.

In another example, provided herein is a method of treating a renal caner (e.g., RCC) in a patient in need thereof, the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the presence of at least one copy of HLA-Bw4 and at least one copy of KIR3DL1 in the patient's genome.

In another example, provided herein is a PD-1 axis binding antagonist (e.g., atezolizumab) for use in treating a renal caner (e.g., RCC) in a patient in need thereof, the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist; and (b) administering an effective amount of a treatment regimen comprising a PD-1 axis binding antagonist to the patient based on the presence of at least one copy of HLA-Bw4 and at least one copy of KIR3DL1 in the patient's genome.

In another example, provided herein is a method of identifying a patient having a renal caner (e.g., RCC) who may benefit from a treatment regimen comprising a PD-1 axis binding antagonist (e.g., atezolizumab), the method comprising determining whether the patient's genome comprises at least one copy of HLA-C1, wherein the presence of at least one copy of HLA-C1 in the patient's genome identifies the patient as one who may benefit from treatment with a treatment regimen comprising a PD-1 axis binding antagonist. In some instances, the method further comprises determining whether the patient's genome comprises at least one copy of KIR2DL3.

In another example, provided herein is a method of identifying a patient having a a renal caner (e.g., RCC) who may benefit from a treatment regimen comprising a PD-1 axis binding antagonist (e.g., atezolizumab), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-C1, wherein the presence of at least one copy of HLA-C1 in the patient's genome identifies the patient as one who may benefit from treatment with a treatment regimen comprising a PD-1 axis binding antagonist. In some instances, the method further comprises determining whether the patient's genome comprises at least one copy of KIR2DL3.

In another example, provided herein is a method of identifying a patient having a renal caner (e.g., RCC) who may benefit from a treatment regimen comprising a PD-1 axis binding antagonist (e.g., atezolizumab), the method comprising determining whether the patient's genome comprises at least one copy of HLA-C1 and at least one copy of KIR2DL3, wherein the presence of at least one copy of HLA-C1 and at least one copy of KIR2DL3 in the patient's genome identifies the patient as one who may benefit from treatment with a treatment regimen comprising a PD-1 axis binding antagonist.

In another example, provided herein is a method of identifying a patient having a a renal caner (e.g., RCC) who may benefit from a treatment regimen comprising a PD-1 axis binding antagonist (e.g., atezolizumab), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-C1 and at least one copy of KIR2DL3, wherein the presence of at least one copy of HLA-C1 and at least one copy of KIR2DL3 in the patient's genome identifies the patient as one who may benefit from treatment with a treatment regimen comprising a PD-1 axis binding antagonist.

In another example, provided herein is a method of identifying a patient having a renal caner (e.g., RCC) who may benefit from a treatment regimen comprising a PD-1 axis binding antagonist (e.g., atezolizumab), the method comprising determining whether the patient's genome comprises at least one copy of HLA-Bw4, wherein the presence of at least one copy of HLA-Bw4 in the patient's genome identifies the patient as one who may benefit from treatment with a treatment regimen comprising a PD-1 axis binding antagonist. In some instances, the method further comprises determining whether the patient's genome comprises at least one copy of KIR3DL1.

In another example, provided herein is a method of identifying a patient having a a renal caner (e.g., RCC) who may benefit from a treatment regimen comprising a PD-1 axis binding antagonist (e.g., atezolizumab), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-Bw4, wherein the presence of at least one copy of HLA-Bw4 in the patient's genome identifies the patient as one who may benefit from treatment with a treatment regimen comprising a PD-1 axis binding antagonist. In some instances, the method further comprises determining whether the patient's genome comprises at least one copy of KIR3DL1.

In another example, provided herein is a method of identifying a patient having a renal caner (e.g., RCC) who may benefit from a treatment regimen comprising a PD-1 axis binding antagonist (e.g., atezolizumab), the method comprising determining whether the patient's genome comprises at least one copy of HLA-Bw4 and at least one copy of KIR3DL1, wherein the presence of at least one copy of HLA-Bw4 and at least one copy of KIR3DL1 in the patient's genome identifies the patient as one who may benefit from treatment with a treatment regimen comprising a PD-1 axis binding antagonist.

In another example, provided herein is a method of identifying a patient having a a renal caner (e.g., RCC) who may benefit from a treatment regimen comprising a PD-1 axis binding antagonist (e.g., atezolizumab), the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising a PD-1 axis binding antagonist by determining whether the patient's genome comprises at least one copy of HLA-Bw4 and at least one copy of KIR3DL1, wherein the presence of at least one copy of HLA-Bw4 and at least one copy of KIR3DL1 in the patient's genome identifies the patient as one who may benefit from treatment with a treatment regimen comprising a PD-1 axis binding antagonist.

In another example, provided herein is a method for selecting a therapy for a patient having a renal caner (e.g., RCC), the method comprising: (a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist (e.g., atezolizumab); and (b) selecting a treatment regimen comprising a PD-1 axis binding antagonist based on the presence of at least one copy of HLA-C1 in the patient's genome. In some instances, the method further comprises determining whether the patient's genome comprises at least one copy of KIR2DL3.

In another example, provided herein is a method for selecting a therapy for a patient having a renal caner (e.g., RCC), the method comprising: (a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist (e.g., atezolizumab); and (b) selecting a treatment regimen comprising a PD-1 axis binding antagonist based on the presence of at least one copy of HLA-C1 and at least one copy of KIR2DL3 in the patient's genome.

In another example, provided herein is a method for selecting a therapy for a patient having a renal caner (e.g., RCC), the method comprising: (a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist (e.g., atezolizumab); and (b) selecting a treatment regimen comprising a PD-1 axis binding antagonist based on the presence of at least one copy of HLA-Bw4 in the patient's genome. In some instances, the method further comprises determining whether the patient's genome comprises at least one copy of KIR3DL1.

In another example, provided herein is a method for selecting a therapy for a patient having a renal caner (e.g., RCC), the method comprising: (a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist (e.g., atezolizumab); and (b) selecting a treatment regimen comprising a PD-1 axis binding antagonist based on the presence of at least one copy of HLA-Bw4 and at least one copy of KIR3DL1 in the patient's genome.

Any of the preceding examples may further include administering an effective amount of a treatment regimen comprising a PD-1 axis binding antagonist (e.g., atezolizumab) to the patient.

In any of the examples described herein, the presence of HLA-C1, HLA-Bw4, KIR2DL3, and/or KIR3DL1 in the patient's genome may be determined using any suitable approach. For example, in some instances, the presence of HLA-C1, HLA-Bw4, KIR2DL3, and/or KIR3DL1 in the patient's genome is determined using next-generation sequencing, Sanger sequencing, a polymerase chain reaction (PCR)-based assay, or a single nucleotide polymorphism (SNP) array. In some instances, the next-generation sequencing comprises germline whole-genome sequencing or germline whole-exome sequencing. In some instances, the PCR-based assay comprises quantitative PCR (qPCR), typing using sequence-specific primers (SSP), or typing using sequence specific oligonucleotide probes (SSO).

In another example, provided herein is a method of treating a renal caner (e.g., RCC) in a patient in need thereof who has been determined to have an increased level of natural killer (NK) cell infiltration in a tumor sample obtained from the patient relative to a reference level of NK cell infiltration, the method comprising administering to the patient an effective amount of a treatment regimen comprising a PD-1 axis binding antagonist (e.g., atezolizumab).

In another example, provided herein is a PD-1 axis binding antagonist (e.g., atezolizumab) for use in treating a renal caner (e.g., RCC) in a patient in need thereof who has been determined to have an increased level of NK cell infiltration in a tumor sample obtained from the patient relative to a reference level of NK cell infiltration.

In another example, provided herein is a method of treating a renal caner (e.g., RCC) in a patient in need thereof, the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist (e.g., atezolizumab); and (b) administering an effective amount of a treatment regimen comprising a PD-1 axis binding antagonist to the patient based on the increased level of NK cell infiltration in the tumor sample obtained from the patient relative to the reference level of NK cell infiltration.

In another example, provided herein is a PD-1 axis binding antagonist (e.g., atezolizumab) for use in a method of treating a renal caner (e.g., RCC) in a patient in need thereof, the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist; and (b) administering an effective amount of a treatment regimen comprising a PD-1 axis binding antagonist to the patient based on the increased level of NK cell infiltration in the tumor sample obtained from the patient relative to the reference level of NK cell infiltration.

In another example, provided herein is a method of identifying a patient having a renal caner (e.g., RCC) who may benefit from a treatment regimen comprising a PD-1 axis binding antagonist (e.g., atezolizumab), the method comprising determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist.

In another example, provided herein is a method of identifying a patient having a a renal caner (e.g., RCC) who may benefit from a treatment regimen comprising a PD-1 axis binding antagonist (e.g., atezolizumab), the method comprising: (a) contacting a tumor sample obtained from the patient with one or more antibodies or nucleotide probes that bind to one or more NK cell markers to determine the level of NK cell infiltration in the tumor sample; and (b) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist.

In another example, provided herein is a method for selecting a therapy for a patient having a renal caner (e.g., RCC), the method comprising: (a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist (e.g., atezolizumab); and (b) selecting a treatment regimen comprising a PD-1 axis binding antagonist based on the increased level of NK cell infiltration in the tumor sample obtained from the patient relative to the reference level of NK cell infiltration.

Any of the examples described herein may include administering an effective amount of a treatment regimen comprising a PD-1 axis binding antagonist (e.g., atezolizumab) to the patient.

In any of the examples described herein, the level of NK cell infiltration may be determined using any suitable approach. For example, in some instances, the level of NK cell infiltration is determined by determining an expression level of an NK cell gene signature, by counting a number of NK cells in the tumor sample, or by detecting the presence or level of one or more NK cell markers, e.g., by immunofluorescence, immunohistochemistry, western blot, flow cytometry, or any other suitable approach.

In any of the examples described herein, the NK cell gene signature may include one or more (e.g., 1, 2, 3, 4, 5, 6, 7, 8, 9, 10, 11, 12, 13, 14, 15, 16, 17, 18, 19, or all 20) of the following genes: CD160, CD244, CTSW, FASLG, GZMA, GZMB, GZMH, IL18RAP, IL2RB, KIR2DL4, KLRB1, KLRC3, KLRD1, KRLF1, KLRK1, NCR1, NKG7, PRF1, XCL1, and XCL2. In some instances, the NK cell gene signature comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of the genes. In some instances, the reference level of NK cell infiltration is a median level. In some instances, the median level is a median level in a population of renal cancer (e.g., RCC) patients.

In another example, provided herein is a method of treating a renal caner (e.g., RCC) in a patient in need thereof whose genome has been determined to lack KIR2DL3 or KIR3DL1, the method comprising administering to the patient an effective amount of a treatment regimen comprising an NK cell-directed therapy agent.

In another example, provided herein is a NK cell-directed therapy agent for use in treating a renal caner (e.g., RCC) in a patient in need thereof whose genome has been determined to lack KIR2DL3 or KIR3DL1.

In another example, provided herein is a method of treating a renal caner (e.g., RCC) in a patient in need thereof, the method comprising: (a) determining whether the patient's genome lacks KIR2DL3 or KIR3DL1, wherein the absence of KIR2DL3 or KIR3DL1 in the patient's genome indicates that the patient is likely to benefit from a treatment regimen comprising an NK cell-directed therapy agent; and (b) administering an effective amount of a treatment regimen comprising an NK cell-directed therapy agent to the patient based on the absence of KIR2DL3 or KIR3DL1 in the patient's genome.

In another example, provided herein is a NK cell-directed therapy agent for use in a method of treating a renal caner (e.g., RCC) in a patient in need thereof, the method comprising: (a) determining whether the patient's genome lacks KIR2DL3 or KIR3DL1, wherein the absence of KIR2DL3 or KIR3DL1 in the patient's genome indicates that the patient is likely to benefit from a treatment regimen comprising an NK cell-directed therapy agent; and (b) administering an effective amount of a treatment regimen comprising an NK cell-directed therapy agent to the patient based on the absence of KIR2DL3 or KIR3DL1 in the patient's genome.

In another example, provided herein is a method of identifying a patient having a renal caner (e.g., RCC) who may benefit from a treatment regimen comprising an NK cell-directed therapy agent, the method comprising determining whether the patient's genome lacks KIR2DL3 or KIR3DL1, wherein the absence of KIR2DL3 or KIR3DL1 in the patient's genome identifies the patient as one who may benefit from treatment with a treatment regimen comprising an NK cell-directed therapy agent.

In another example, provided herein is a method of identifying a patient having a a renal caner (e.g., RCC) who may benefit from a treatment regimen comprising an NK cell-directed therapy agent, the method comprising: (a) performing germline WGS or WES by fragmenting a DNA sample obtained from the patient to produce fragmented DNA, adding adapters to the fragmented DNA to produce one or more libraries, and sequencing the one or more libraries; and (b) identifying the patient as one who may benefit from a treatment regimen comprising an NK cell-directed therapy agent by determining whether KIR2DL3 or KIR3DL1 are absent from the patient's genome, wherein the absence of KIR2DL3 or KIR3DL1 in the patient's genome identifies the patient as one who may benefit from treatment with a treatment regimen comprising an NK cell-directed therapy agent.

In another example, provided herein is a method for selecting a therapy for a patient having a renal caner (e.g., RCC), the method comprising: (a) determining whether the patient's genome lacks KIR2DL3 or KIR3DL1, wherein the absence of KIR2DL3 or KIR3DL1 in the patient's genome identifies the patient as one who may benefit from treatment with a treatment regimen comprising an NK cell-directed therapy agent; and (b) selecting a treatment regimen comprising NK cell-directed therapy agent based on the absence of KIR2DL3 or KIR3DL1 in the patient's genome.

Any suitable NK cell directed therapy may be used, including any NK cell-directed therapy described herein (see, e.g., Section V below). In some examples, any NK cell-directed therapy described in Hodgins et al. J. Clin. Invest. 129(9):3499-3510, 2019, may be used. In some instances, the NK cell-directed therapy agent comprises allogeneic NK cells, autologous NK cells, off-the-shelf NK cells, chimeric antigen receptor (CAR)-NK cells, cytokine therapy, an NK cell engager (e.g., a bispecific killer cell engager (BiKE), a tri-specific killer cell engager (TriKE), or a tetra-specific killer cell engager (TetraKE)), an NK cell checkpoint receptor antagonist, or an oncolytic virus.

In some examples, the NK cell-directed therapy agent comprises allogeneic NK cells, autologous NK cells, off-the-shelf NK cells, or a combination thereof. In some instances, the NK cell-directed therapy agent comprises allogeneic NK cells. In other instances, the NK cell-directed therapy agent comprises autologous NK cells. In yet other instances, the NK cell-directed therapy agent comprises off-the-shelf NK cells.

In some examples, the allogeneic NK cells, the autologous NK cells, or the off-the-shelf NK cells are engineered to express KIR2DL3 or KIR3DL1. For example, in some instances, the allogeneic NK cells, the autologous NK cells, or the off-the-shelf NK cells are engineered to express KIR2DL3. For example, in other instances, the allogeneic NK cells, the autologous NK cells, or the off-the-shelf NK cells are engineered to express KIR3DL1.

In some examples, the patient's genome comprises at least one copy (e.g., 1 or 2 copies) of HLA-C1 or at least one copy (e.g., 1 or 2 copies) of HLA-Bw4. In some instances, the patient's genome comprises at least one copy (e.g., 1 or 2 copies) of HLA-C1. In some instances, the patient's genome comprises at least one copy (e.g., 1 or 2 copies) of HLA-Bw4.

In some examples, treatment with the allogeneic NK cells, the autologous NK cells, or the off-the-shelf NK cells engineered to express KIR2DL3 or KIR3DL1 results in the patient being one who may benefit from treatment with a treatment regimen comprising a PD-1 axis binding antagonist (e.g., atezolizumab).

Any of the preceding examples may further include administering a treatment regimen comprising a PD-1 axis binding antagonist (e.g., atezolizumab) to the patient, e.g., before, concurrently, or after treatment with a treatment regimen comprising an NK cell-directed therapy agent.

In some examples, the benefit is in terms of improved overall survival (OS) or improved progression-free survival (PFS). In some instances, the benefit is in terms of improved OS. In some instances, the benefit is in terms of improved PFS. In some instances, improvement is relative to treatment with a treatment regimen that does not comprise the PD-1 axis binding antagonist.

In any of the examples described herein, the patient may be chemotherapy-naïve.

In any of the examples described herein, the treatment regimen may be a first-line treatment regimen.

Any suitable PD-1 axis binding antagonist may be used, including any PD-1 axis binding antagonist described herein (see, e.g., Section IV below). In some examples, the PD-1 axis binding antagonist is selected from a PD-L1 binding antagonist, a PD-1 binding antagonist, and a PD-L2 binding antagonist.

In some examples, the PD-1 axis binding antagonist is a PD-L1 binding antagonist. In some instances, the PD-L1 binding antagonist is an anti-PD-L1 antibody. In some instances, the anti-PD-L1 antibody comprises (a) a hypervariable region (HVR)-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively. In some instances, the anti-PD-L1 antibody comprises (a) a VH comprising the amino acid sequence of SEQ ID NO: 9, and (b) a VL comprising the amino acid sequence of SEQ ID NO: 10. In some instances, the anti-PD-L1 antibody is atezolizumab, durvalumab, avelumab, or MDX-1105. In some instances, the anti-PD-L1 antibody is atezolizumab. In some instances, the anti-PD-L1 antibody is administered intravenously or subcutaneously. In some instances, the atezolizumab is administered intravenously every two weeks at a dose of 840 mg. In some instances, the atezolizumab is administered intravenously every three weeks at a dose of 1200 mg. In some instances, the atezolizumab is administered intravenously every four weeks at a dose of 1680 mg.

In other examples, the PD-1 axis binding antagonist is a PD-1 binding antagonist. In some instances, the PD-1 binding antagonist is an anti-PD-1 antibody. In some instances, the anti-PD-1 antibody is nivolumab, pembrolizumab, MEDI-0680, spartalizumab, cemiplimab, camrelizumab, sintilimab, tislelizumab, toripalimab, or dostarlimab.

In some examples, the PD-1 axis binding antagonist is administered in combination with an effective amount of one or more additional therapeutic agents. For example, in some embodiments, the treatment regimen includes a taxane (e.g., nab-paclitaxel or paclitaxel), a platinum-based chemotherapeutic agent (e.g., carboplatin), an anti-angiogenic agent (e.g., an anti-VEGF antibody such as atezolizumab), a NK cell-directed therapy (e.g., an NK cell engager), or a combination thereof.

In some examples, the treatment regimen further comprises a taxane (e.g., nab-paclitaxel or paclitaxel). In some instances, the taxane is nab-paclitaxel. In some instances, the taxane is paclitaxel.

In some examples, the treatment regimen further comprises a platinum-based chemotherapeutic agent. In some instances, the platinum-based chemotherapeutic agent is carboplatin.

In some examples, the treatment regimen further comprises an anti-angiogenic agent. In some instances, the anti-angiogenic agent is an anti-VEGF antibody. In some instances, the anti-VEGF antibody is bevacizumab.

Any of the examples described herein may further include administering an additional therapeutic agent to the patient. In some instances, the additional therapeutic agent is selected from the group consisting of an immunotherapy agent, a cytotoxic agent, a growth inhibitory agent, a radiation therapy agent, an anti-angiogenic agent, and combinations thereof. In some instances, the immunotherapy agent is an NK cell-directed agent, including any NK cell-directed agent described herein.

In any of the preceding examples, each dosing cycle may have any suitable length, e.g., about 7 days, about 14 days, about 21 days, about 28 days, or longer. In some instances, each dosing cycle is about 21 days.

The patient is preferably a human.

As a general proposition, the therapeutically effective amount of a PD-1 axis binding antagonist (e.g., atezolizumab) administered to a human will be in the range of about 0.01 to about 50 mg/kg of patient body weight, whether by one or more administrations.

In some exemplary embodiments, the PD-1 axis binding antagonist is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example.

In one instance, a PD-1 axis binding antagonist is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg. In some instances, the PD-1 axis binding antagonist may be administered at a dose of about 1000 mg to about 1400 mg every three weeks (e.g., about 1100 mg to about 1300 mg every three weeks, e.g., about 1150 mg to about 1250 mg every three weeks).

In some instances, a patient is administered a total of 1 to 50 doses of a PD-1 axis binding antagonist, e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In particular instances, the doses may be administered intravenously.

In some instances, atezolizumab is administered to the patient intravenously at a dose of about 840 mg every 2 weeks, about 1200 mg every 3 weeks, or about 1680 mg of every 4 weeks. In some instances, atezolizumab is administered to the patient intravenously at a dose of 1200 mg every 3 weeks.

The PD-1 axis binding antagonist and/or any additional therapeutic agent(s) (e.g., a taxane (e.g., nab-paclitaxel or paclitaxel), a platinum-based chemotherapeutic agent (e.g., carboplatin), an anti-angiogenic agent (e.g., an anti-VEGF antibody such as bevacizumab), and/or an NK cell-directed therapy (e.g., an NK cell engager)) may be administered in any suitable manner known in the art. For example, the PD-1 axis binding antagonist and/or any additional therapeutic agent(s) may be administered sequentially (on different days) or concurrently (on the same day or during the same treatment cycle). In some instances, the PD-1 axis binding antagonist is administered prior to the additional therapeutic agent. In other instances, the PD-1 axis binding antagonist is administered after the additional therapeutic agent. In some instances, the PD-1 axis binding antagonist and/or any additional therapeutic agent(s) may be administered on the same day. In some instances, the PD-1 axis binding antagonist may be administered prior to an additional therapeutic agent that is administered on the same day. For example, the PD-1 axis binding antagonist may be administered prior to chemotherapy on the same day. In another example, the PD-1 axis binding antagonist may be administered prior to both chemotherapy and another drug (e.g., bevacizumab) on the same day. In other instances, the PD-1 axis binding antagonist may be administered after an additional therapeutic agent that is administered on the same day. In yet other instances, the PD-1 axis binding antagonist is administered at the same time as the additional therapeutic agent. In some instances, the PD-1 axis binding antagonist is in a separate composition as the additional therapeutic agent. In some instances, the PD-1 axis binding antagonist is in the same composition as the additional therapeutic agent. In some instances, the PD-1 axis binding antagonist is administered through a separate intravenous line from any other therapeutic agent administered to the patient on the same day.

The PD-1 axis binding antagonist and any additional therapeutic agent(s) may be administered by the same route of administration or by different routes of administration. In some instances, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some instances, the additional therapeutic agent is administered intravenously, intramuscularly, subcutaneously, topically, orally, transdermally, intraperitoneally, intraorbitally, by implantation, by inhalation, intrathecally, intraventricularly, or intranasally.

In a preferred embodiment, the PD-1 axis binding antagonist is administered intravenously. In one example, atezolizumab may be administered intravenously over 60 minutes; if the first infusion is tolerated, all subsequent infusions may be delivered over 30 minutes. In some examples, the PD-1 axis binding antagonist is not administered as an intravenous push or bolus. In some examples, the taxane (e.g., nab-paclitaxel or paclitaxel), the platinum-based chemotherapeutic agent (e.g., carboplatin), he anti-angiogenic agent (e.g., an anti-VEGF antibody such as bevacizumab), and/or the NK cell-directed therapy (e.g., an NK cell engager) is administered intravenously.

In some examples, the NSCLC is metastatic non-squamous NSCLC, and the treatment regimen comprises atezolizumab, nab-paclitaxel, and carboplatin. In some instances, atezolizumab is administered as an intravenous (IV) infusion at a dose of 1200 mg on Day 1 of each 21-day cycle; nab-paclitaxel is administered as an IV infusion at a dose of 100 mg/m² on Days 1, 8, and 15 of each 21-day cycle; and carboplatin is administered at an area under the concentration curve (AUC) of 6 mg/mL/min on Day 1 of each 21-day cycle.

In some examples, the NSCLC is metastatic non-squamous NSCLC, and the treatment regimen comprises atezolizumab, paclitaxel, and carboplatin. In some instances, atezolizumab is administered as an IV infusion at a dose of 1200 mg on Day 1 of each 21-day cycle; paclitaxel is administered as an IV infusion at a dose of 200 mg/m² on Day 1 each 21-day cycle; and carboplatin is administered at an AUC of 6 mg/mL/min on Day 1 of each 21-day cycle.

In some examples, the NSCLC is metastatic non-squamous NSCLC, and the treatment regimen comprises atezolizumab, bevacizumab, paclitaxel, and carboplatin. In some instances, atezolizumab is administered as an IV infusion at a dose of 1200 mg on Day 1 of each 21-day cycle; bevacizumab is administered as an IV infusion at a dose of 15 mg/kg on Day 1 of each 21-day cycle; paclitaxel is administered as an IV infusion at a dose of 200 mg/m² on Day 1 each 21-day cycle; and carboplatin is administered at an AUC of 6 mg/mL/min on Day 1 of each 21-day cycle.

In some examples, the NSCLC is metastatic squamous NSCLC, and the treatment regimen comprises atezolizumab, nab-paclitaxel, and carboplatin. In some instances, atezolizumab is administered as an IV infusion at a dose of 1200 mg on Day 1 of each 21-day cycle; nab-paclitaxel is administered as an IV infusion at a dose of 100 mg/m² on Days 1, 8, and 15 of each 21-day cycle; and carboplatin is administered at an area under the concentration curve (AUC) of 6 mg/mL/min on Day 1 of each 21-day cycle.

In some examples, the NSCLC is metastatic squamous NSCLC, and the treatment regimen comprises atezolizumab, paclitaxel, and carboplatin. In some instances, atezolizumab is administered as an IV infusion at a dose of 1200 mg on Day 1 of each 21-day cycle; paclitaxel is administered as an IV infusion at a dose of 175 mg/m² or 200 mg/m² on Days 1, 8, and 15 of each 21-day cycle; and carboplatin is administered at an AUC of 6 mg/mL/min on Day 1 of each 21-day cycle.

In some examples, the renal cancer is metastatic RCC, and the treatment regimen comprises atezolizumab and bevacizumab. In some instances, atezolizumab is administered as an IV infusion at a dose of 1200 mg on Days 1 and 22 of each 42-day cycle; and bevacizumab is administered as an IV infusion at a dose of 15 mg/mk on Days 1 and 22 of each 42-day cycle.

Also provided herein are methods for treating cancer (e.g., NSCLC) in a patient comprising administering to the patient a treatment regimen comprising an effective amount of a PD-1 axis binding antagonist (e.g., atezolizumab) and/or a taxane (e.g., nab-paclitaxel or paclitaxel), a platinum-based chemotherapeutic agent (e.g., carboplatin), an anti-angiogenic agent (e.g., an anti-VEGF antibody such as bevacizumab), and/or an NK cell-directed therapy (e.g., an NK cell engager) in combination with another anti-cancer agent or cancer therapy. For example, a PD-1 axis binding antagonist may be administered in combination with an additional chemotherapy or chemotherapeutic agent (see definition above); a targeted therapy or targeted therapeutic agent; an immunotherapy or immunotherapeutic agent, for example, a monoclonal antibody; one or more cytotoxic agents (see definition above); or combinations thereof. For example, the PD-1 axis binding antagonist may be administered in combination with bevacizumab, paclitaxel, paclitaxel protein-bound (e.g., nab-paclitaxel), carboplatin, cisplatin, pemetrexed, gemcitabine, etoposide, cobimetinib, vemurafenib, or a combination thereof. The PD-1 axis binding antagonist may be an anti-PD-L1 antibody (e.g., atezolizumab) or an anti-PD-1 antibody.

For example, when administering with chemotherapy with or without bevacizumab, atezolizumab may be administered at a dose of 1200 mg every 3 weeks prior to chemotherapy and bevacizumab. In another example, following completion of 4-6 cycles of chemotherapy, and if bevacizumab is discontinued, atezolizumab may be administered at a dose of 840 mg every 2 weeks, 1200 mg every 3 weeks, or 1680 mg every four weeks. In another example, atezolizumab may be administered at a dose of 840 mg, followed by 100 mg/m² of paclitaxel protein-bound (e.g., nab-paclitaxel); for each 28 day cycle, atezolizumab is administered on days 1 and 15, and paclitaxel protein-bound is administered on days 1, 8, and 15. In another example, when administering with carboplatin and etoposide, atezolizumab can be administered at a dose of 1200 mg every 3 weeks prior to chemotherapy. In yet another example, following completion of 4 cycles of carboplatin and etoposide, atezolizumab may be administered at a dose of 840 mg every 2 weeks, 1200 mg every 3 weeks, or 1680 mg every 4 weeks. In another example, following completion of a 28 day cycle of cobimenitib and vemurafenib, atezolizumab may be administered at a dose of 840 mg every 2 weeks with cobimetinib at a dose of 60 mg orally once daily (21 days on, 7 days off) and vemurafenib at a dose of 720 mg orally twice daily.

In some instances, the treatment may further comprise an additional therapy. Any suitable additional therapy known in the art or described herein may be used. The additional therapy may be radiation therapy, surgery, gene therapy, DNA therapy, viral therapy, RNA therapy, immunotherapy, bone marrow transplantation, nanotherapy, monoclonal antibody therapy, gamma irradiation, or a combination of the foregoing.

In some instances, the additional therapy is the administration of side-effect limiting agents (e.g., agents intended to lessen the occurrence and/or severity of side effects of treatment, such as anti-nausea agents, a corticosteroid (e.g., prednisone or an equivalent, e.g., at a dose of 1-2 mg/kg/day), hormone replacement medicine(s), and the like).

### III. Assessment of PD-L1 Expression

The expression of PD-L1 may be assessed in a patient treated according to any of the methods and compositions for use described herein. The methods and compositions for use may include determining the expression level of PD-L1 in a biological sample (e.g., a tumor sample) obtained from the patient. In other examples, the expression level of PD-L1 in a biological sample (e.g., a tumor sample) obtained from the patient has been determined prior to initiation of treatment or after initiation of treatment.

PD-L1 expression may be determined using any suitable approach. For example, PD-L1 expression may be determined as described in U.S. Patent Application Publication Nos. US 2018/0030138 and US 2018/0037655, which are incorporated by reference herein in their entirety. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the patient, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1 L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263.

In some examples, a tumor sample obtained from the patient has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the patient has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some examples, tumor samples may be scored for PD-L1 positivity in tumor-infiltrating immune cells and/or in tumor cells according to the criteria for diagnostic assessment shown in Table 1 and/or Table 2, respectively.

TABLE 1
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any | |
| intensity in tumor-infiltrating immune cells | |
| covering <1% of tumor area occupied by tumor cells, | |
| associated intratumoral stroma, and contiguous | |
| peri-tumoral desmoplastic stroma | |
| Presence of discernible PD-L1 staining of any | IC1 |
| intensity in tumor-infiltrating immune cells | |
| covering ≥1% to <5% of tumor area occupied by tumor cells, | |
| associated intratumoral stroma, and contiguous | |
| peri-tumoral desmoplastic stroma | |
| Presence of discernible PD-L1 staining of any | IC2 |
| intensity in tumor-infiltrating immune cells | |
| covering ≥5% to <10% of tumor area occupied by tumor | |
| cells, associated intratumoral stroma, and | |
| contiguous peri-tumoral desmoplastic stroma | |
| Presence of discernible PD-L1 staining of any | IC3 |
| intensity in tumor-infiltrating immune cells | |
| covering ≥10% of tumor area occupied by tumor cells, | |
| associated intratumoral stroma, and contiguous | |
| peri-tumoral desmoplastic stroma | |

TABLE 2
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any | |
| intensity in <1% of tumor cells | |
| Presence of discernible PD-L1 staining of any | TC1 |
| intensity in ≥1% to <5% of tumor cells | |
| Presence of discernible PD-L1 staining of any | TC2 |
| intensity in ≥5% to <50% of tumor cells | |
| Presence of discernible PD-L1 staining of any | TC3 |
| intensity in ≥50% of tumor cells | |

### IV. PD-1 Axis Binding Antagonists

PD-1 axis binding antagonists may include PD-L1 binding antagonists, PD-1 binding antagonists, and PD-L2 binding antagonists. Any suitable PD-1 axis binding antagonist may be used.

A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)2 fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody comprises:
(a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and
(b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In one embodiment, the anti-PD-L1 antibody comprises:
(a) a heavy chain variable region VH comprising the amino acid sequence:

(SEQ ID NO: 9)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,

and
(b) the light chain variable region (VL) comprising the amino acid sequence:

(SEQ ID NO: 10)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 9; (b) a VL comprising an amino acid sequence comprising having at least 95% sequence identity (e.g., at least 95%, 96%, 97%, 98%, or 99% sequence identity) to, or the sequence of SEQ ID NO: 10; or (c) a VH as in (a) and a VL as in (b).

In one embodiment, the anti-PD-L1 antibody comprises atezolizumab, which comprises:
(a) the heavy chain amino acid sequence:

(SEQ ID NO: 1)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG,

and
(b) the light chain amino acid sequence:

(SEQ ID NO: 2)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US 20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

B. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DCIg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances the anti-PD-1 antibody is a monoclonal antibody. In some instances, the anti-PD-1 antibody is an antibody fragment selected from the group consisting of Fab, Fab′, Fab′-SH, Fv, scFv, and (Fab′)₂ fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US 20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

C. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

### V. NK Cell-Directed Therapies

Provided herein are methods for treating cancer (e.g., NSCLC) in a patient comprising administering to the patient a treatment regimen comprising an NK cell-directed therapy agent. Also provided are related compositions (e.g., pharmaceutical compositions) for use, kits, and articles of manufacture. Any of the methods, compositions for use, kits, or articles of manufacture described herein may include or involve any of the agents described below.

Any suitable NK cell-directed therapy agent may be used. In some examples, any NK cell-directed therapy described in Hodgins et al. J. Clin. Invest. 129(9):3499-3510, 2019, may be used. In some instances, the NK cell-directed therapy agent comprises allogeneic NK cells, autologous NK cells, off-the-shelf NK cells, chimeric antigen receptor (CAR)-NK cells, cytokine therapy, an NK cell engager (e.g., a bispecific killer cell engager (BiKE), a tri-specific killer cell engager (TriKE), or a tetra-specific killer cell engager (TetraKE)), a NK cell checkpoint receptor antagonist, an NK cell checkpoint receptor antagonist, or an oncolytic virus. In some instances, the NK cell-directed therapy agent comprises adoptive cell transfer (e.g., with allogeneic NK cells, autologous NK cells, off-the-shelf NK cells, or chimeric antigen receptor (CAR)-NK cells).

In some examples, the NK cell-directed therapy agent comprises allogeneic NK cells, autologous NK cells, off-the-shelf NK cells, or a combination thereof. In some instances, the NK cell-directed therapy agent comprises allogeneic NK cells. In other instances, the NK cell-directed therapy agent comprises autologous NK cells. In yet other instances, the NK cell-directed therapy agent comprises off-the-shelf NK cells.

Exemplary NK cells that may be used include, without limitation, FT500 (a universal, off-the-shelf NK cell cancer immunotherapy derived from a clonal master iPSC line; see, e.g., Cichocki et al. Sci. Trans. Med. 12(568):eaaz5618, 2020), FT516 (a universal, off-the-shelf NK cell cancer immunotherapy derived from a clonal master iPSC line engineered to express a high-affinity 158V, non-cleavable CD16 (hnCD16) Fc receptor, which has been modified to prevent its down-regulation and to enhance its binding to tumor-targeting antibodies; see, e.g., Zhu et al. Blood 135(6):399-410, 2020), FT536 (a universal, off-the-shelf NK cell cancer immunotherapy derived from a clonal master engineered iPSC line that includes four functional modifications: a CAR that targets the α3 domain of MICA and MICB; a high-affinity 158V, non-cleavable CD16 (hnCD16) Fc receptor that augments ADCC; an IL-15 receptor fusion (IL-15RF) that promotes enhanced NK cell activity; and the elimination of CD38 expression which enhances NK cell metabolic fitness, persistence and anti-tumor functionality; see, e.g., de Andrade et al. Cancer Immunol. Res. 8:769-80, 2020), FT596 (a universal, off-the-shelf NK cell cancer immunotherapy derived from a clonal master iPSC line engineered with three anti-tumor functional modalities: a CAR that targets B-cell antigen CD19; a high-affinity 158V, non-cleavable CD16 (hnCD16) Fc receptor, which has been modified to prevent its down-regulation and to enhance its binding to tumor-targeting antibodies; and an IL-15 receptor fusion (IL-15RF) that promotes enhanced NK cell activity; see, e.g., Liu et al. New Engl. J. Med. 382:545-53, 2020), FT538 (a universal, off-the-shelf NK cell cancer immunotherapy derived from a clonal master iPSC line that incorporates three functional modifications: a high-affinity 158V, non-cleavable CD16 (hnCD16) Fc receptor, which has been modified to augment ADCC; an IL-15 receptor fusion (IL-15RF) that promotes enhanced NK cell activity; and the elimination of CD38 expression to mitigate the potential for NK cell fratricide), FT573 (a universal, off-the-shelf NK cell cancer immunotherapy derived from a clonal master engineered iPSC line that incorporates four functional modifications: a CAR that targets B7H3; a high-affinity 158V, non-cleavable CD16 (hnCD16) Fc receptor that augments ADCC; an IL-15 receptor fusion (IL-15RF) that promotes enhanced NK cell activity; and the elimination of CD38 expression which enhances NK cell metabolic fitness, persistence and anti-tumor functionality), and FT576 (a universal, off-the-shelf NK cell cancer immunotherapy derived from a clonal master iPSC line that incorporates four functional modifications: a CAR that targets BCMA; a high-affinity 158V, non-cleavable CD16 (hnCD16) Fc receptor, which has been modified to augment ADCC; an IL-15 receptor fusion (IL-15RF) that promotes enhanced NK cell activity; and the elimination of CD38 expression to mitigate the potential for NK cell fratricide), from Fate Therapeutics.

In some examples, the NK cells (e.g., allogeneic NK cells, the autologous NK cells, or the off-the-shelf NK cells) are engineered to express KIR2DL3 or KIR3DL1. For example, in some instances, the allogeneic NK cells, the autologous NK cells, or the off-the-shelf NK cells are engineered to express KIR2DL3. For example, in other instances, the allogeneic NK cells, the autologous NK cells, or the off-the-shelf NK cells are engineered to express KIR3DL1. The NK cells (e.g., allogeneic NK cells, the autologous NK cells, or the off-the-shelf NK cells) may be engineered to express KIR2DL3 or KIR3DL1 using any suitable approach, including gene editing or transduction (e.g., lentiviral transduction).

In some examples, the allogeneic NK cells are derived from a cell line, e.g., NK92 or KyHG1. In other examples, the allogeneic NK cells may be derived from cord blood or iPSCs.

In some examples, the NK cell-directed therapy agent is a natural killer cell transduced with a chimeric antigen receptor (CAR-NK; also referred to as NAR-T). In some aspects, the chimeric antigen receptor (CAR) comprises an antigen-binding domain (e.g., an antibody or a fragment thereof; a T-cell receptor (TCR) or a fragment thereof) binding to a tumor antigen (e.g., a tumor antigen of Table 3), a transmembrane domain, and one or more intracellular signaling domains, e.g., a primary signaling domain (e.g., CD3ζ and/or a costimulatory signaling domain (e.g., CD28, 4-1BB) (WO2017-114497; Hartmann et al., EMBO Molecular Medicine, 9(9), 2017). The intracellular signaling domain may act to activate cytotoxicity.

In some examples, the CAR is introduced into a population of NK cells. The population of NK cells may be prepared for CAR, e.g., by use of a flow-through module, as described in WO 2017/117112. The NK cells may be autologous, e.g., deriving from the patient, or allogenic, e.g., derived from a donor. In some aspects, CAR-NK cells are introduced to a patient intravenously or intratumorally.

TABLE 3
Exemplary Tumor Antigens

| Tumor antigen | Accession number |
|---|---|
| BMPR1B (bone morphogenetic protein receptor-type IB) | Genbank accession no. NM_001203 |
| E16 (LAT1, SLC7A5) | Genbank accession no. NM_003486 |
| STEAP1 (six transmembrane epithelial antigen of | Genbank accession no. NM_012449 |
| prostate) | |
| 0772P (CA125, MUC16) | Genbank accession no. AF361486 |
| MPF (MPF, MSLN, SMR, megakaryocyte potentiating | Genbank accession no. NM_005823 |
| factor, mesothelin) | |
| Napi2b (Napi3b, NAPI-3B, NPTIIb, SLC34A2, solute | Genbank accession no. NM_006424 |
| carrier family 34 (sodium phosphate), member 2, type II | |
| sodium-dependent phosphate transporter 3b | |
| Sema 5b (F1110372, KIAA1445, Mm.42015, SEMA5B, | Genbank accession no. AB040878 |
| SEMAG, Semaphorin 5b H log, sema domain, seven | |
| thrombospondin repeats (type 1 and type 1-like), | |
| transmembrane domain (TM) and short cytoplasmic | |
| domain, (semaphorin) 5B | |
| PSCA hlg (2700050C12Rik, C530008O16Rik, RIKEN | Genbank accession no. AY358628 |
| cDNA 2700050C12, RIKEN cDNA 2700050C12 gene | |
| ETBR (Endothelin type B receptor) | Genbank accession no. AY275463 |
| MSG783 (RNF124, hypothetical protein FLJ20315) | Genbank accession no. NM_017763 |
| STEAP2 (HGNC_8639, IPCA-1, PCANAP1, STAMP1, | Genbank accession no. AF455138 |
| STEAP2, STMP, prostate cancer associated gene 1, | |
| prostate cancer associated protein 1, six transmembrane | |
| epithelial antigen of prostate 2, six transmembrane | |
| prostate protein) | |
| TrpM4 (BR22450, FLJ20041, TRPM4, TRPM4B, | Genbank accession no. NM_017636 |
| transient receptor potential cation channel, subfamily M, | |
| member 4) | |
| CRIPTO (CR, CR1, CRGF, CRIPTO, TDGF1, | Genbank accession no. NP_003203 or |
| teratocarcinoma-derived growth factor) | NM_003212 |
| CD21 (CR2 (Complement receptor 2) or C3DR | Genbank accession no. M26004 |
| (C3d/Epstein Barr virus receptor) or Hs.73792) | |
| CD79b (CD79B, CD79β, IGb (immunoglobulin- | Genbank accession no. NM_000626 or |
| associated beta), B29 | 11038674 |
| FcRH2 (IFGP4, IRTA4, SPAP1A (SH2 domain | Genbank accession no. NM_030764, |
| containing phosphatase anchor protein 1a), SPAP1B, | AY358130 |
| SPAP1C) | |
| HER2 (ErbB2) | Genbank accession no. M11730 |
| NCA (CEACAM6) | Genbank accession no. M18728 |
| MDP (DPEP1) | Genbank accession no. BC017023 |
| IL20Rα (IL20Rα, ZCYTOR7) | Genbank accession no. AF184971 |
| Brevican (BCAN, BEHAB) | Genbank accession no. AF229053 |
| EphB2R (DRT, ERK, HekS, EPHT3, TyroS) | Genbank accession no. NM_004442 |
| ASLG659 (B7h) | Genbank accession no. AX092328 |
| PSCA (Prostate stem cell antigen precursor) | Genbank accession no. AJ297436 |
| GEDA | Genbank accession No. AY260763 |
| BAFF-R (B cell-activating factor receptor, BLyS receptor | Genbank accession No. AF116456 |
| 3, BR3) | |
| CD22 (B-cell receptor CD22-B isoform, BL-CAM, Lyb-8, | Genbank accession No. AK026467 |
| Lyb8, SIGLEC-2, FLJ22814) | |
| CD79a (CD79A, CD79α, immunoglobulin-associated | Genbank accession No. NP_001774.10 |
| alpha) | |
| CXCRS (Burkitt's lymphoma receptor) | Genbank accession No. NP_001707.1 |
| HLA-DOB (Beta subunit of MHC class II molecule (Ia | Genbank accession No. NP_002111.1 |
| antigen)) | |
| P2X5 (Purinergic receptor P2X ligand-gated ion channel | Genbank accession No. NP_002552.2 |
| 5) | |
| CD72 (B-cell differentiation antigen CD72, Lyb-2) | Genbank accession No. NP_001773.1 |
| LY64 (Lymphocyte antigen 64 (RP105), type I | Genbank accession No. NP_005573.1 |
| membrane protein of the leucine rich repeat (LRR) | |
| family) | |
| FcRH1 (Fc receptor-like protein 1) | Genbank accession No. NP_443170.1 |
| IRTA2 (Immunoglobulin superfamily receptor | Genbank accession No. Human: AF343662, |
| translocation associated 2) | AF343663, AF343664, AF343665, |
| | AF369794, AF397453, AK090423, |
| | AK090475, AL834187, AY358085; |
| | Mouse: AK089756, AY158090, AY506558; |
| | NP_112571.1. |
| TENB2 (TMEFF2, tomoregulin, TPEF, HPP1, TR, | Genbank accession No. AF179274; |
| putative transmembrane proteoglycan) | AY358907, CAF85723, CQ782436 |
| PMEL17 (silver homolog; SILV; D12S53E; PMEL17; SI; | McGlinchey, R. P. et al (2009) Proc. Natl. |
| SIL) | Acad. Sci. U.S.A. 106 (33), 13731-13736; |
| | Kummer, M. P. et al (2009) J. Biol. Chem. |
| | 284 (4), 2296-2306; |
| TMEFF1 (transmembrane protein with EGF-like and two | Harms, P. W. (2003) Genes Dev. 17 (21), |
| follistatin-like domains 1; Tomoregulin-1) | 2624-2629; Gery, S. et al (2003) Oncogene |
| | 22 (18): 2723-2727; |
| GDNF-Ra1 (GDNF family receptor alpha 1; GFRA1; | Kim, M. H. et al (2009) Mol. Cell. Biol. 29 (8), |
| GDNFR; GDNFRA; RETL1; TRNR1; RET1L; GDNFR- | 2264-2277; Treanor, J. J. et al (1996) Nature |
| alpha1; GFR-ALPHA-1) | 382 (6586): 80-83; |
| Ly6E (lymphocyte antigen 6 complex, locus E; Ly67, | de Nooij-van Dalen, A. G. et al (2003) Int. J. |
| RIG-E, SCA-2, TSA-1) | Cancer 103 (6), 768-774; Zammit, D. J. et al |
| | (2002) Mol. Cell. Biol. 22 (3): 946-952; WO |
| | 2013/17705 |
| TMEM46 (shisa homolog 2 (Xenopus laevis); SHISA2) | Furushima, K. et al (2007) Dev. Biol. 306 (2), |
| | 480-492; Clark, H. F. et al (2003) Genome |
| | Res. 13 (10): 2265-2270; |
| Ly6G6D (lymphocyte antigen 6 complex, locus G6D; | Mallya, M. et al (2002) Genomics 80 (1): 113- |
| Ly6-D, MEGT1) | 123; Ribas, G. et al (1999) J. Immunol. 163 |
| | (1): 278-287; |
| LGR5 (leucine-rich repeat-containing G protein-coupled | Salanti, G. et al (2009) Am. J. Epidemiol. 170 |
| receptor 5; GPR49, GPR67) | (5): 537-545; Yamamoto, Y. et al (2003) |
| | Hepatology 37 (3): 528-533; |
| RET (ret proto-oncogene; MEN2A; HSCR1; MEN2B; | Tsukamoto, H. et al (2009) Cancer Sci. 100 |
| MTC1; PTC; CDHF12; Hs.168114; RET51; RET-ELE1) | (10): 1895-1901; Narita, N. et al (2009) |
| | Oncogene 28 (34): 3058-3068; |
| LY6K (lymphocyte antigen 6 complex, locus K; LY6K; | Ishikawa, N. et al (2007) Cancer Res. 67 |
| HSJ001348; FLJ35226 | (24): 11601-11611; de Nooij-van Dalen, A. G. |
| | et al (2003) Int. J. Cancer 103 (6): 768-774; |
| GPR19 (G protein-coupled receptor 19; Mm.4787) | Montpetit, A. and Sinnett, D. (1999) Hum. |
| | Genet. 105 (1-2): 162-164; O'Dowd, B. F. et al |
| | (1996) FEBS Lett. 394 (3): 325-329; |
| GPR54 (KISS1 receptor; KISS1R; GPR54; HOT7T175; | Navenot, J. M. et al (2009) Mol. Pharmacol. |
| AXOR12) | 75 (6): 1300-1306; Hata, K. et al (2009) |
| | Anticancer Res. 29 (2): 617-623; |
| ASPHD1 (aspartate beta-hydroxylase domain containing | Gerhard, D. S. et al (2004) Genome Res. 14 |
| 1; LOC253982) | (10B): 2121-2127; |
| Tyrosinase (TYR; OCAIA; OCA1A; tyrosinase; SHEP3) | Bishop, D. T. et al (2009) Nat. Genet. 41 |
| | (8): 920-925; Nan, H. et al (2009) Int. J. |
| | Cancer 125 (4): 909-917; |
| TMEM118 (ring finger protein, transmembrane 2; | Clark, H. F. et al (2003) Genome Res. 13 |
| RNFT2; FLJ14627) | (10): 2265-2270; Scherer, S. E. et al (2006) |
| | Nature 440 (7082): 346-351 |
| GPR172A (G protein-coupled receptor 172A; GPCR41; | Ericsson, T. A. et al (2003) Proc. Natl. Acad. |
| FLJ11856; D15Ertd747e); NP_078807.1; NM_024531.3 | Sci. U.S.A. 100 (11): 6759-6764; Takeda, S. |
| | et al (2002) FEBS Lett. 520 (1-3): 97-101. |
| CD33 | Sabbath et al., (1985) J. Clin. Invest. 75: 756- |
| | 56; Andrews et al., (1986) Blood 68: 1030-5 |
| CLL-1 (CLEC12A, MICL, and DCAL2) | Drickamer K (1999) Curr. Opin. Struct. Biol. 9 |
| | (5): 585-90; van Rhenen A, et al., (2007) |
| | Blood 110 (7): 2659-66; Chen C H, et al. |
| | (2006) Blood 107 (4): 1459-67; Marshall A S, |
| | et al. (2006) Eur. J. Immunol. 36 (8): 2159-69; |
| | Bakker A B, et al (2005) Cancer Res. 64 |
| | (22): 8443-50; Marshall A S, et al (2004) J. |
| | Biol. Chem. 279 (15): 14792-802). |

In some examples, the NK cell-directed therapy agent is an NK cell engager (e.g., a bispecific killer cell engager (BiKE), a tri-specific killer cell engager (TriKE), or a tetra-specific killer cell engager (TetraKE)). In some instances, the NK cell engager binds to one or more targets (e.g., proteins, e.g., receptors) on the surface of an NK cell (e.g., CD16, NKG2D, a SLAM family protein, NKp30, NKp44, or NKp46) and one or more targets (e.g., proteins, e.g., receptors) on the surface of the tumor cell (e.g., a tumor antigen, including CD30, CD33, EGFR, BCMA, or any tumor antigen described in Table 3). Exemplary NK cell engagers are described, e.g., in WO 2019/198051; Reusch et al., mAbs, 6(3):727-738; 2014; U.S. Pat. No. 7,129,330B1; U.S. Pat. No. 9,035,026B2; Wo0111059A1; Treder et al., Journal of Clinical Oncology, 34(15 suppl), 2016; and Ellwanger et al., J Immunother Cancer, 3(Suppl 2): 219, 2015. In some embodiments, the NK cell engager is a nanoparticle-based NK cell engager, e.g., a nanoparticle-based trispecific NK cell engager (nano-TriNKE) (see, e.g., Au et al. Science Advances 6(27):eaba8564, 2020. Exemplary NK cell engagers include, e.g., IPH6101 (Innate Pharma/Sanofi).

An NK cell engager may be multispecific, e.g., bispecific, trispecific, or tetraspecific.

An NK cell engager may be multivalent for a particular target, e.g., bivalent, trivalent, tetravalent, pentavalent, or hexavalent.

In some examples, the NK cell engager is a bispecific NK cell engager comprising a first targeting domain binding to an epitope on a NK cell and a second targeting domain binding to a different target, e.g., a tumor antigen. In some aspects, the bispecific NK cell engager comprises a first targeting domain binding CD16a, a protein expressed on the surface of NK cells, and a second targeting domain binding the tumor marker CD30. In some aspects, the bispecific NK cell engager comprises a first targeting domain binding CD16a and a second targeting domain binding epidermal growth factor receptor (EGFR) or EGFRvIII. In some aspects, the bispecific NK cell engager comprises a first targeting domain binding NKp46 and a second targeting domain binding a tumor antigen, e.g., a tumor antigen listed in Table 3.

In some instances, any NK cell engager described in WO 2019/198051, which is incorporated herein by reference in its entirety, may be used.

Any suitable NK cell checkpoint receptor antagonist may be used. Exemplary, non-limiting examples of NK cell checkpoint receptor antagonists include, e.g., a KIR antagonist (e.g., an anti-KIR antibody, such as lirumab (IPH2102), which targets KIR2DL1-3 and KIR2DS1-2), a CD94/NKG2A antagonist (e.g., an anti-CD94 antibody or protein expression blocker (PEBL) or an anti-NKG2A antibody (e.g., monalizumab (IPH2201) or PEBL), a CTLA-4 antagonist (e.g., an anti-CTLA-4 antibody), a PD-1 axis binding antagonist, a LAG3 antagonist (e.g., an anti-LAG3 antibody), or a TIM-3 antagonist (e.g., an anti-TIM-3 antibody).

Any suitable cytokine therapy may be used. For example, the cytokine therapy may include type 1 interferon, a TLR agonist, or a cGAS/STING agonist, IL-2, IL-12, IL-18, IL-15, combinations thereof, or variants thereof (e.g., engineered IL-2 cytokine “super-2” or engineered IL-15 cytokine ALT-803).

Any suitable oncolytic virus can be used, e.g., any oncolytic virus described in Hodgins et al. supra.

### VII. Pharmaceutical Compositions and Formulations

Also provided herein are pharmaceutical compositions and formulations comprising a PD-1 axis binding antagonist (e.g., atezolizumab) and, optionally, a pharmaceutically acceptable carrier. The disclosure also provides pharmaceutical compositions and formulations comprising a taxane (e.g., nab-paclitaxel or paclitaxel), a platinum-based chemotherapeutic agent (e.g., carboplatin), an anti-angiogenic agent (e.g., bevacizumab), and/or a NK cell-directed therapy agent (e.g., an NK cell engager), and optionally, a pharmaceutically acceptable carrier.

Pharmaceutical compositions and formulations as described herein can be prepared by mixing the active ingredients (e.g., a PD-1 axis binding antagonist) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions.

An exemplary atezolizumab formulation comprises glacial acetic acid, L-histidine, polysorbate 20, and sucrose, with a pH of 5.8. For example, atezolizumab may be provided in a 20 mL vial containing 1200 mg of atezolizumab that is formulated in glacial acetic acid (16.5 mg), L-histidine (62 mg), polysorbate 20 (8 mg), and sucrose (821.6 mg), with a pH of 5.8. In another example, atezolizumab may be provided in a 14 mL vial containing 840 mg of atezolizumab that is formulated in glacial acetic acid (11.5 mg), L-histidine (43.4 mg), polysorbate 20 (5.6 mg), and sucrose (575.1 mg) with a pH of 5.8.

### VIII. Articles of Manufacture or Kits

In another aspect, provided herein is an article of manufacture or a kit comprising a PD-1 axis binding antagonist (e.g., atezolizumab) and/or a taxane (e.g., nab-paclitaxel or paclitaxel), a platinum-based chemotherapeutic agent (e.g., carboplatin), an anti-angiogenic agent (e.g., bevacizumab), and/or a NK cell-directed therapy agent (e.g., an NK cell engager). In some instances, the article of manufacture or kit further comprises package insert comprising instructions for using the PD-1 axis binding antagonist to treat or delay progression of cancer (e.g., NSCLC) in a patient. In some instances, the article of manufacture or kit further comprises package insert comprising instructions for using the PD-1 axis binding antagonist in combination with a taxane (e.g., nab-paclitaxel or paclitaxel), a platinum-based chemotherapeutic agent (e.g., carboplatin), an anti-angiogenic agent (e.g., bevacizumab), and/or a NK cell-directed therapy agent (e.g., an NK cell engager) to treat or delay progression of cancer in a patient. Any of the PD-1 axis binding antagonists and/or taxanes, platinum-based chemotherapeutic agents, anti-angiogenic agents, and/or NK cell-directed therapy agents described herein may be included in the article of manufacture or kits.

In another aspect, provided herein is an article of manufacture or a kit comprising a NK cell-directed therapy agent (e.g., an NK cell engager). In some instances, the article of manufacture or kit further comprises package insert comprising instructions for using the NK cell-directed therapy agent to treat or delay progression of cancer (e.g., NSCLC) in a patient.

In some instances, the PD-1 axis binding antagonist and the additional therapeutic agents (e.g., a taxane (e.g., nab-paclitaxel or paclitaxel), a platinum-based chemotherapeutic agent (e.g., carboplatin), an anti-angiogenic agent (e.g., bevacizumab), and/or a NK cell-directed therapy agent (e.g., an NK cell engager)) are in the same container or separate containers. Suitable containers include, for example, bottles, vials, bags, and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agents include, for example, bottles, vials, bags and syringes.

Any of the articles of manufacture or kits may include instructions to administer a PD-1 axis binding antagonist and/or a taxane (e.g., nab-paclitaxel or paclitaxel), a platinum-based chemotherapeutic agent (e.g., carboplatin), an anti-angiogenic agent (e.g., bevacizumab), and/or a NK cell-directed therapy agent (e.g., an NK cell engager) to a patient in accordance with any of the methods described herein, e.g., any of the methods set forth in Section II above.

In another example, provided herein is an article of manufacture comprising a PD-1 axis binding antagonist and instructions to administer the PD-1 axis binding antagonist for treatment of cancer (e.g., NSCLC) in a patient in need thereof whose genome has been determined to comprise at least one copy of HLA-C1. In some instances, the patient's genome further comprises at least one copy of KIR2DL3.

In another example, provided herein is an article of manufacture comprising a PD-1 axis binding antagonist and instructions to administer the PD-1 axis binding antagonist for treatment of cancer (e.g., NSCLC) in a patient in need thereof whose genome has been determined to comprise at least one copy of HLA-C1 and at least one copy of KIR2DL3.

In another example, provided herein is an article of manufacture comprising a PD-1 axis binding antagonist and instructions to administer the PD-1 axis binding antagonist for treatment of cancer (e.g., NSCLC) in a patient in need thereof whose genome has been determined to comprise at least one copy of HLA-Bw4. In some instances, the patient's genome further comprises at least one copy of KIR3DL1.

In another example, provided herein is an article of manufacture comprising a PD-1 axis binding antagonist and instructions to administer the PD-1 axis binding antagonist for treatment of treating cancer (e.g., NSCLC) in a patient in need thereof whose genome has been determined to comprise at least one copy of HLA-Bw4 and at least one copy of KIR3DL1.

In another example, provided herein is an article of manufacture comprising a PD-1 axis binding antagonist and instructions to administer the PD-1 axis binding antagonist for treatment of cancer (e.g., NSCLC) in a patient in need thereof who has been determined to have an increased level of NK cell infiltration in a tumor sample obtained from the patient relative to a reference level of NK cell infiltration.

In another example, provided herein is an article of manufacture comprising an NK cell-directed therapy agent and instructions to administer the NK cell-directed therapy agent for treatment of cancer (e.g., NSCLC) in a patient in need thereof whose genome has been determined to lack KIR2DL3 or KIR3DL1.

### EXAMPLES

### Example 1. Immunogenetics Variation Involved in NK Cell Education and NK Cell Infiltration is Associated with Outcome in Non-Small Cell Lung Cancer Patients Treated with Immune Checkpoint Blockade

a. Introduction

Natural Killer (NK) cells are important contributors to antitumor immune responses. Along with NK cell abundance in the tumor, diverse tumor immune evasion strategies targeting NK cells, and differential distribution of NK cell subsets across different tissue types, the immunogenetic composition of patients' genomes is considered to be an important determinant of NK cell effectiveness.

In particular, genetic variation in human leukocyte antigen (HLA) and killer-cell immunoglobulin-like receptor (KIR) genes impacts NK cell education and function. NK cell education is a dynamic process to achieve functional maturation and self-tolerance, and better NK cell education results in stronger response to “missing self” phenotypes. Allele-specific interactions of inhibitory KIR and HLA proteins contribute to NK cell education (Pende et al., Front. Immunol., 10: Article 1179, 2019). KIR3DL1+ NK cells from Bw4/Bw4 donors have been shown to display increased responsiveness (IFNy production) to MHC-deficient tumors (Kim et al., PNAS, 105(8): 3053-3058, 2008). KIR2DL3 and KIR3DL1 predominantly occur on KIR A haplotypes, which in general are associated with improved response to pathogens (Jamil and Khakoo, J Biomed Biotechnol, 2011: Article ID 298348, 2011).

b. Methods

HLA allelic variation and KIR gene presence were inferred using germline whole-genome sequencing data from 1,395 patients across three atezolizumab (anti-PD-L1) clinical trials (IMpower130, IMpower131, IMpower150) in non-small cell lung cancer (NSCLC). IMpower130 (NCT02367781) investigated the safety and efficacy of a treatment regimen comprising atezolizumab, nab-paclitaxel, and carboplatin in metastatic non-squamous NSCLC as compared to a control treatment without atezolizumab. IMpower131 (NCT02367794) investigated the safety and efficacy of a treatment regimen comprising atezolizumab, paclitaxel, and carboplatin or atezolizumab, nab-paclitaxel, and carboplatin in metastatic squamous NSCLC as compared to a control treatment without atezolizumab. IMpower150 (NCT02367794) investigated the safety and efficacy of a treatment regimen comprising atezolizumab, paclitaxel, and carboplatin or atezolizumab, bevacizumab, paclitaxel, and carboplatin in metastatic non-squamous NSCLC as compared to a control treatment without atezolizumab. Patient numbers for the atezolizumab (atezo) and control arms included in the germline genetic analysis are shown in Table 4.

TABLE 4
Patient populations for germline genetics analyses

| Study | Atezo arms | Control arms |
|---|---|---|
| IMpower150 | 433 | 182 |
| IMpower130 | 202 | 102 |
| IMpower131 | 320 | 156 |

HLA alleles were computationally inferred from germline whole-genome sequencing data (30× coverage) using the software HLA-HD (Kawaguchi et al., Hum Mutat., 38(7):788-797, 2017). HLA alleles are described, e.g., at The IPD and IMGT/HLA database (Robinson et al., Nucleic Acids Research 43: D423-431, 2015).

KIR gene presence was computationally inferred from germline whole-genome sequencing data (30× coverage) using the software KPI (Roe et al. Front. Immunol., 11: 583013, 2020). KIR genes are variable in terms of copy number, and individuals can carry them on 0,1, or both of their chromosomes. The software method used identified presence or absence of a certain KIR gene (0 vs. 1/2) in an individual.

Association analyses were first performed on the study level. Cox proportional-hazard models were used to investigate associations of genotypes or a gene score (median cut for high/low definition) with overall or progression-free survival.

The metagen function in the meta package for R was used for meta-analyses (e.g., fixed effect and random effects meta-analysis based on estimates and their standard errors). The inverse variance method was used for pooling.

As detailed in sections c-f below, the results indicated a significant role of both NK cell genotypes and degree of NK cell infiltration in patient responses to anti-PD-L1 cancer immunotherapy.

c. HLA-KIR Interactions are Associated with Outcome of Atezolizumab Treatment in NSCLC

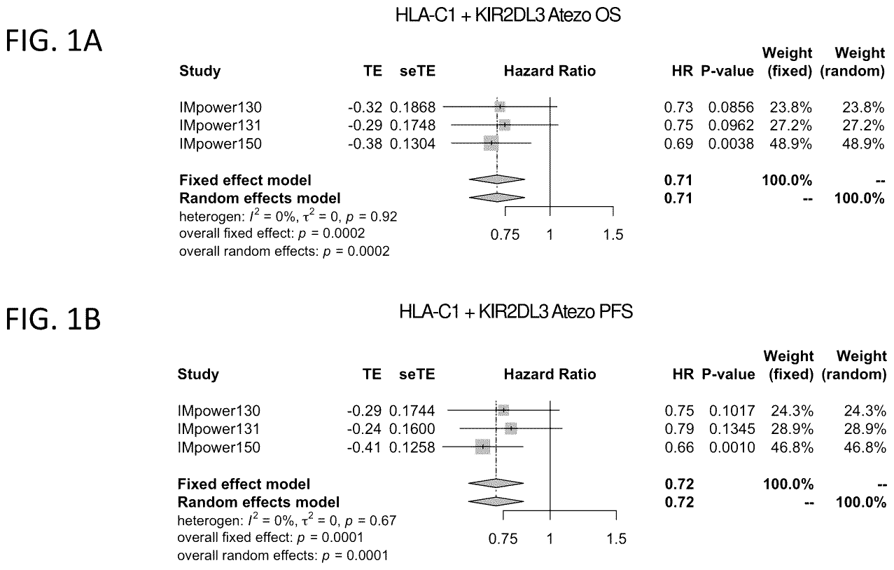

Patients treated with atezolizumab who carried at least one copy of both K/R2DL3 and its ligand HLA-C1 had longer overall survival (OS) and progression-free survival (PFS) compared to patients without this NK-cell educating interaction (N=955, HR=0.71, p=0.0002) (FIGS. 1A and 1B). No associations were observed in the control arms of the trials.

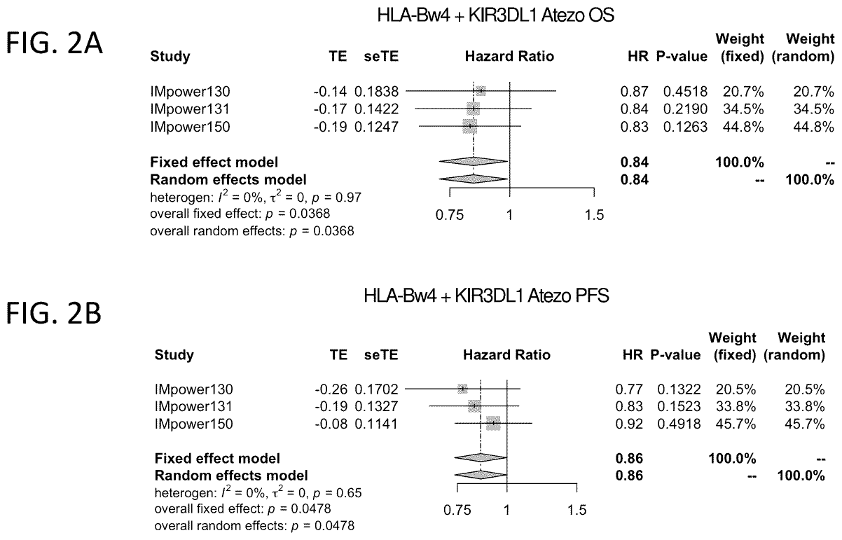

Similarly, patients treated with atezolizumab who carried at least one copy of both K/R3DL1 and its ligand HLA-Bw4 had longer OS and PFS compared to patients without this interaction (HR=0.84, p=0.04) (FIGS. 2A and 2B). No significant associations could be found in the chemotherapy control arms of the trials (N=440).

d. HLA-C1 Carrier Status is Associated with Outcome of Atezolizumab Treatment

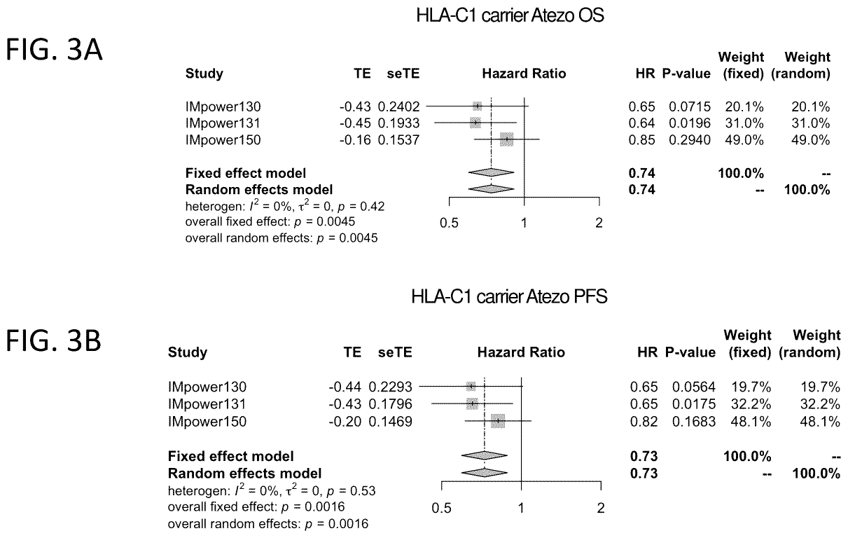

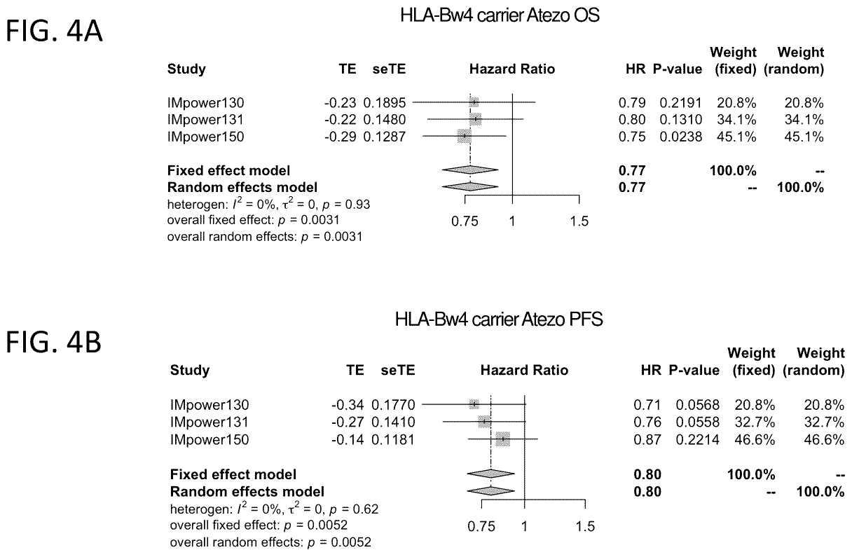

HLA ligand groups defined according to KIR interaction (HLA-C1 carrier status and HLA-Bw4 carrier status) were similarly found to be associated with outcome (PFS and OS) in patients treated with atezolizumab, without consideration of the patient's KIR genotype (FIGS. 3A, 3B, 4A, and 4B).

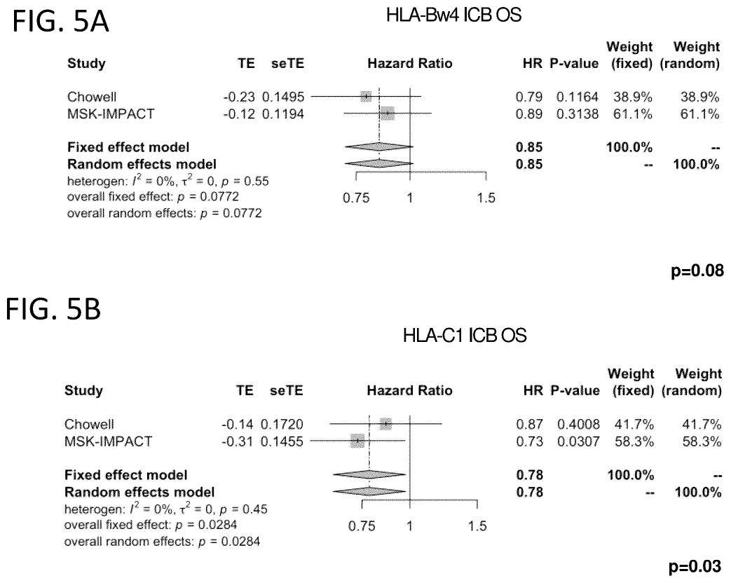

In addition, HLA-C1 carrier status was found to be beneficial for outcome of treatment with immune checkpoint blockade in an analysis of a recently published dataset of melanoma and NSCLC patients (N=1,55, HR=0.74, p=0.01, data from Chowell et al., Science, 359(6375): 582-587, 2018) (FIGS. 5A and 5B).

e. NK Cell Infiltration is Associated with Outcome of Atezolizumab Treatment

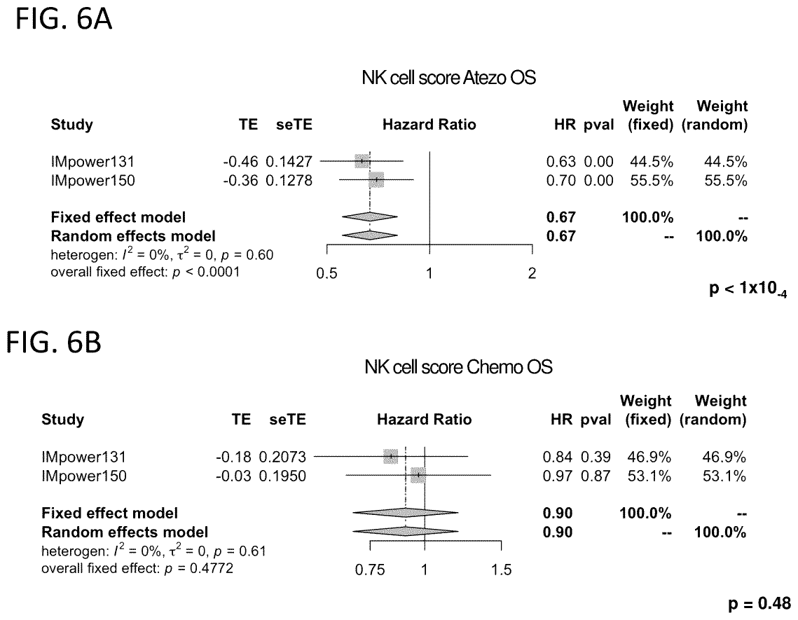

High (above-median) NK cell infiltration was found to be associated with longer OS in patients treated with atezolizumab (N=619, HR=0.75, p=0.01) (FIG. 6A), using a gene signature derived from RNA-sequencing data (NK cell score; Cursons et al., Cancer Immunology Research, 7(7): 1162-1174, 2019). The gene signature comprised 20 genes: CD160, CD244, CTSW, FASLG, GZMA, GZMB, GZMH, IL18RAP, IL2RB, KIR2DL4, KLRB1, KLRC3, KLRD1, KLRF1, KLRK1, NCR1, NKG7, PRF1, XCL1, and XCL2. The gene signature was measured as described in Cursons et al., Cancer Immunology Research, 7(7): 1162-1174, 2019. Association analyses were performed as described in Example 1 b, above. Again, no significant association was found in the control arms (N=288) (FIG. 6B).

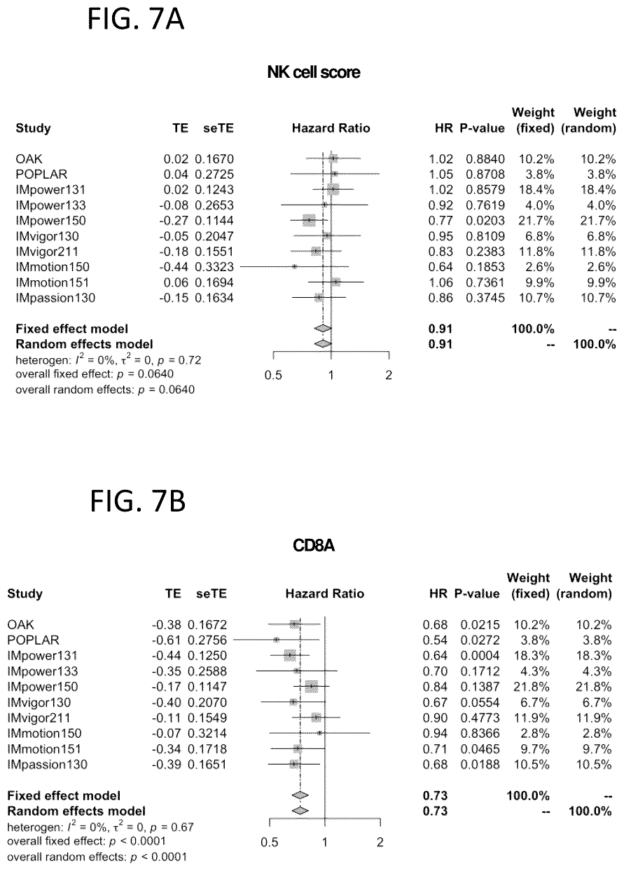

Of note, T cell and NK cell infiltration are correlated (FIGS. 7A and 7B).

Patient numbers for the atezolizumab and control arms included in the NK score RNAseq analyses are shown in Table 5.

TABLE 5
Patient populations for RNAseq analyses

| Study | Atezo arms | Control arms |
|---|---|---|
| IMpower150 | 360 | 151 |
| IMpower131 | 259 | 137 |

### Example 2. Evaluation of Immunogenetic Variation Involved in NK Cell Education and NK Cell Infiltration with Outcome in Renal Cancer Patients Treated with Immune Checkpoint Blockade

a. Methods

HLA allelic variation and KIR gene presence were inferred using germline whole-genome sequencing data from the atezolizumab (anti-PD-L1) clinical trial IMmotion151 as described in Example 1. IMmotion151 (NCT02420821) investigated the safety and efficacy of a treatment regimen comprising atezolizumab and bevacizumab in in participants with inoperable, locally advanced, or metastatic renal cell carcinoma (RCC) as compared to a control treatment comprising sunitinib.

For the NK cell signature analysis, data from IMmotion150 were also included. IMmotion150 (NCT01984242) investigated the safety and efficacy of a treatment regimen comprising atezolizumab and bevacizumab in in participants with inoperable, locally advanced, or metastatic renal cell carcinoma (RCC) as compared to a control treatment comprising sunitinib.

b. Associations Between HLA-KIR Interactions and Outcome of Atezolizumab Treatment in RCC

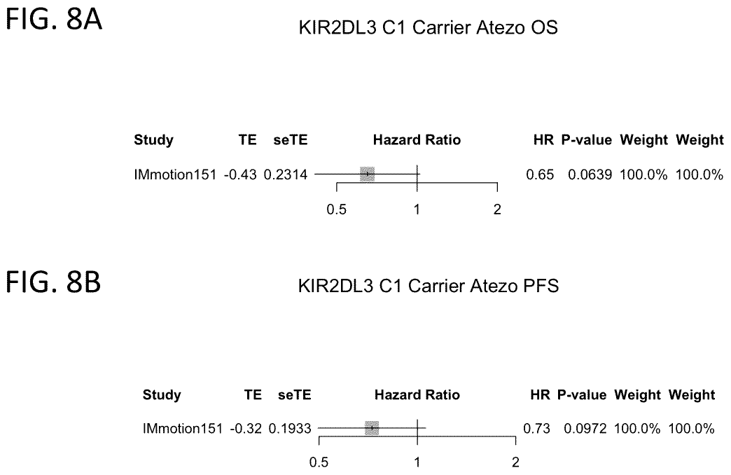

OS and PFS hazard ratios for patients treated with atezolizumab who carried at least one copy of both K/R2DL3 and its ligand HLA-C1 compared to patients without this NK-cell educating interaction are shown in FIGS. 8A and 8B.

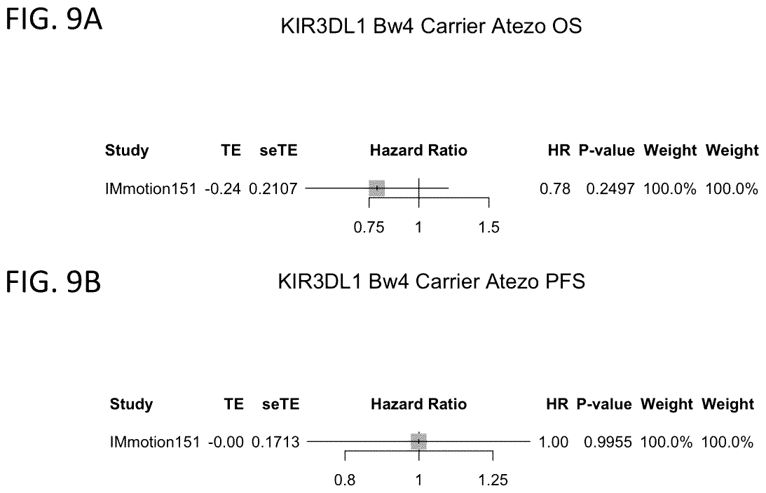

OS and PFS hazard ratios for patients treated with atezolizumab who carried at least one copy of both K/R3DL1 and its ligand HLA-Bw4 compared to patients without this NK-cell educating interaction are shown in FIGS. 9A and 9B.

c. Associations Between HLA-C1 or HLA-Bw4 Carrier Status and Outcome of Atezolizumab Treatment in RCC

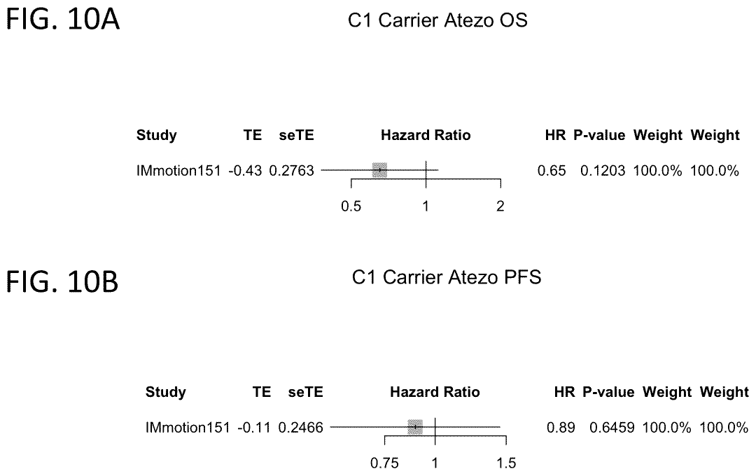

OS and PFS hazard ratios for patients treated with atezolizumab who carried at least one copy of HLA-C1 are shown in FIGS. 10A and 10B.

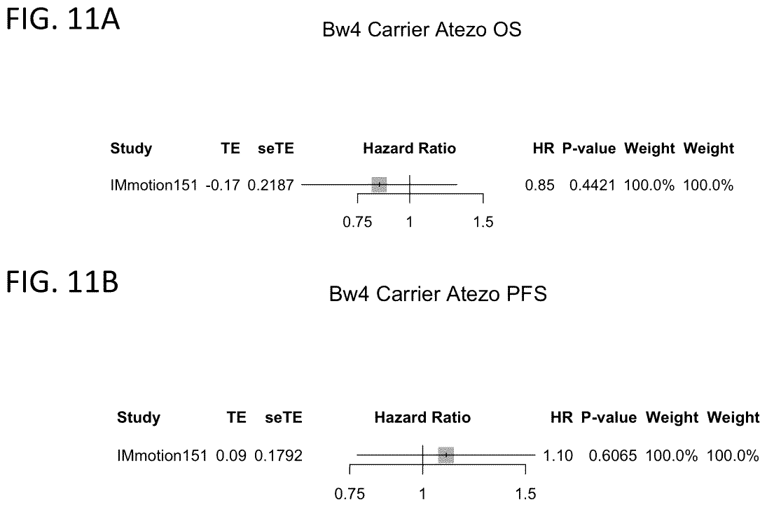

OS and PFS hazard ratios for patients treated with atezolizumab who carried at least one copy of HLA-Bw4 are shown in FIGS. 11A and 11B.

d. Association Between NK Cell Infiltration and Outcome of Atezolizumab Treatment in RCC

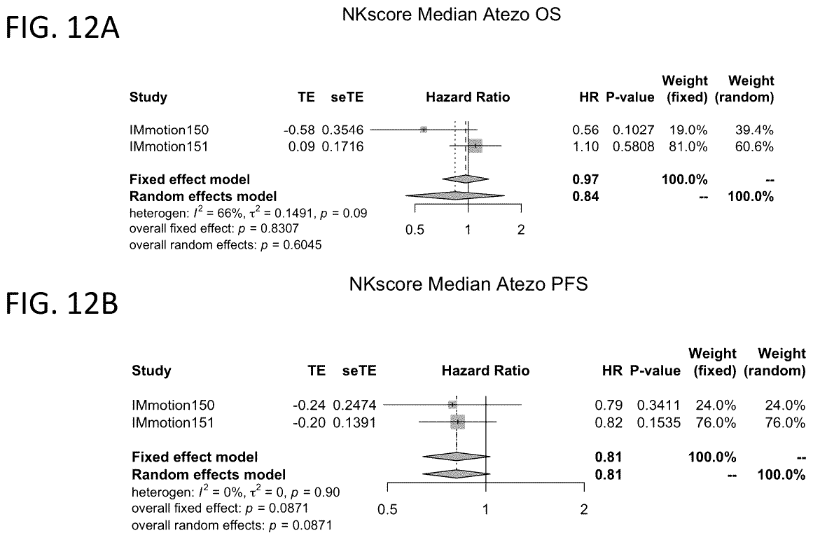

OS and PFS hazard ratios for patients treated with atezolizumab who had a high (above-median) NK cell infiltration score, determined as described in Example 1, are shown in FIGS. 12A and 12B.

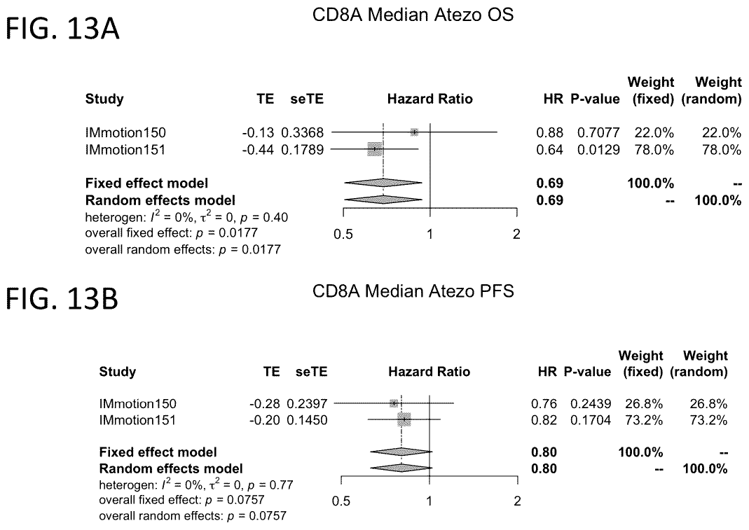

OS and PFS hazard ratios for patients treated with atezolizumab who had a high (above-median) CD8A expression level are shown in FIGS. 13A and 13B.

e. Conclusions

A trend for improved response of RCC patients treated with atezolizumab in the IMmotion151 clinical trial with respect to the HLA-KIR genotypes and degree of NK cell infiltration as described in Example 1 for NSCLC was observed. The effect estimates for RCC were in a similar range as for the NSCLC trials described in Example 1.

### Example 3. Immunogenetic Variation Involved in NK Cell Education and NK Cell Infiltration is Associated with Outcome in Non-Small Cell Lung Cancer Patients Treated with Immune Checkpoint Blockade

Immune-mediated adverse events (imAE) commonly occur in patients treated with immune checkpoint inhibitors (ICI), and pneumonitis is known to occur in 3-5% of patients treated with anti-PD-1/PD-L1 antibodies (Wang et al., Thorac Cancer, 11: 191-197, 2020). Most cases are grade 1 or 2 events and can be treated with immunosuppression, but high-grade events occur in a minority of patients and can be fatal (Naidoo et al., J Clin Oncol, 35: 709-717, 2016). Out of 1761 atezolizumab (anti-PD-L1) treated patients across nine Genentech (GNE) clinical trials with available whole-genome sequencing data, 72 (4.1%) developed pneumonitis (Table 6). The trials included were IMmotion151 (WO29637), IMpassion130 (WO29522), IMpower110 (G029431), IMpower130 (G029537), IMpower131 (G029437), IMpower132 (G029438), IMpower133 (G030081), IMpower150 (G029436), and IMvigor211 (G029294); these studies comprised patients having renal cell carcinoma (RCC), triple-negative breast cancer (TNBC), non-squamous or squamous non-small cell lung cancer (NSCLC), small cell lung cancer (SCLC), and urothelial bladder cancer.

TABLE 6
Investigated cohorts

| | ICI-associated | | Treatment | | | |
|---|---|---|---|---|---|---|
| | pneumonitis | | anti- | anti- | anti- | anti-PD-1 + |
| Cohort | yes | no | PD-L1 | PD-1 | CTLA-4 | anti-CTLA-4 |
| GNE | 72 | 1689 | 1761 | 0 | 0 | 0 |
| PICI | 19 | 19 | 14 | 22 | 0 | 2 |
| PMC | 15 | 149 | 0 | 47 | 14 | 103 |
| Total | 106 | 1857 | 1775 | 69 | 14 | 105 |

GNE, Genentech; PICI, Parker Institute for Cancer Immunotherapy; PMC, Peter MacCallum Cancer Centre.

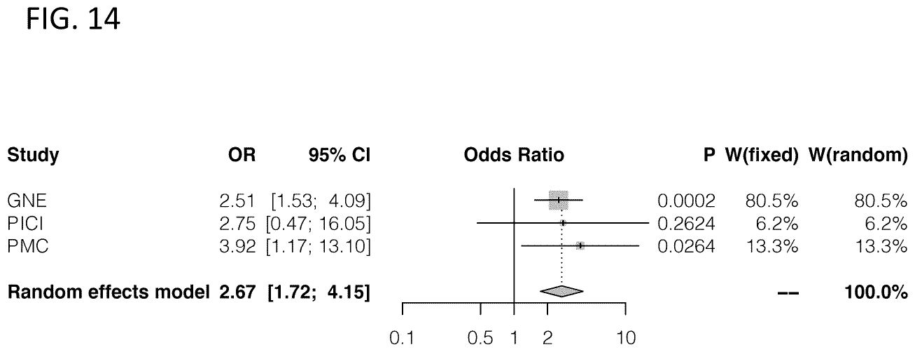

HLA genotypes were inferred using HLA-HD (Kawaguchi et al., Hum Mutat, 38: 788-797, 2017), and an association study was performed including 87 alleles with a carrier frequency of >2%. Two HLA class II alleles that are part of a common haplotype showed significant associations with pneumonitis risk after multiple testing adjustment (HLA-DRB1*15:01, HLA-DQA1*01:02), with HLA-DRB1*15:01 showing the strongest association (p=0.0002, odds ratio (OR)=2.51). No associations were identified in the control arms (N=1192). In order to confirm these results, and to test whether the association is generalizable to different classes of ICI, two additional cohorts were genotyped using an Illumina genome-wide SNP array (GSA v3), followed by HLA imputation using HIBAG (Zheng et al., Pharmacogenomics: 14: 192-200, 2014): the cohorts were (1) 20 ICI-treated cancer patients with pneumonitis and 20 matched controls without pneumonitis from a pilot study on the AEROSMITH trial from the Parker Institute for Cancer Immunotherapy (PICI) and (2) 15 ICI-treated melanoma patients with pneumonitis and 149 without pneumonitis from Peter MacCallum Cancer Centre (PMC) (Table 6). In the PICI pilot cohort, HLA-DRB1*15:01 did not reach statistical significance in spite of a comparable odds ratio (OR) (p=0.26, OR=2.75), but the allele was significantly associated with pneumonitis risk in the PMC cohort (p=0.03, OR=3.92). A meta-analysis across the three cohorts yielded a highly significant p-value of 1.2×10⁻⁵ (OR=2.67, FIG. 14), suggesting that the association is generalizable across ICI. Importantly, the same class II haplotype was previously shown to be associated with diverse lung inflammatory phenotypes, including fibrotic phenotypes (Tian et al., Nat Commun, 8: 599, 2017; Fingerlin et al., BMC Genet., 17: 74, 2016; Voorter et al., Hum Immunol, 66: 826-835, 2005; Furukawa et al., PLoS One, 7: e33133, 2012).

In summary, these findings establish HLA class II allelic variation as a potential risk factor in ICI-associated pneumonitis.

### Example 4. HLA Class II Loss of Heterozygosity (LOH) is Associated with Poor Outcome from Atezolizumab Treatment

The analysis provided in this Example showed that HLA class II loss, but not HLA class I loss, is associated with poor outcome to atezolizumab treatment.

Absence or decrease of HLA expression may be due to genetic or epigenetic modification, or indirect regulation, and may be a result of the tumor evolutionary trajectory to evade anti-tumor immune responses. Antigen-specific signal provided by the binding of a TCR to antigenic peptide complexed with MHC is also referred to as “signal 1.” Most therapeutic approaches in cancer immunology rely on functional antigen presentation, such that a loss or downregulation of HLA expression can be a potent immune evasion strategy for a tumor. In principle, HLA loss or downregulation should be counteracted by NK cells, which may be affected by differential distribution of NK cell subsets across different tissue types, NK cell abundance in the tumor, tumor immune evasion strategies modulating NK cell effectiveness, and the immunogenomic composition of a patient's tumor.

HLA class I and class II LOH was inferred computationally from tumor whole exome sequencing (WES) data from clinical trials in which patients were administered atezolizumab. The trials included were IMpower131 (G029437), IMpower133 (G030081), IMpower150 (G029436), POPLAR (NCT01903993), and IMmotion150 (NCT01984242); these studies comprised patients having NSCLC, SCLC, or mRCC.

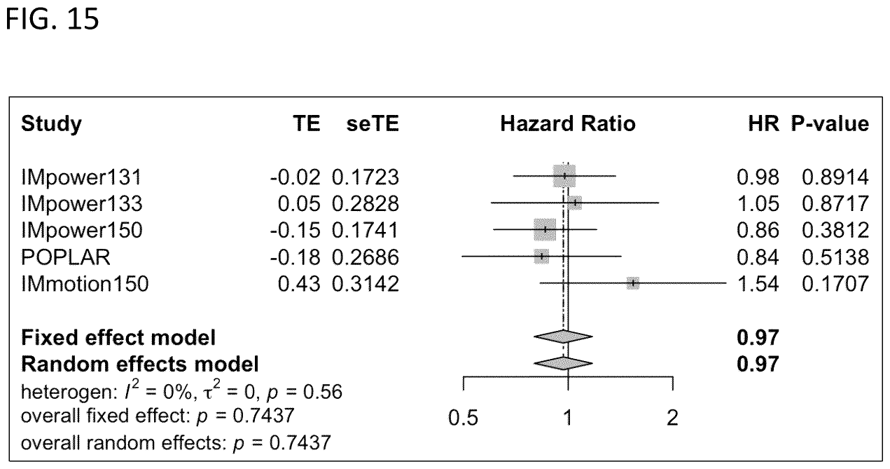

HLA class I LOH was not associated with outcome. Atezolizumab-treated patients with LOH did not show worse overall survival (OS) compared to patients without LOH (FIG. 15). Similar results were obtained for patients with loss of a complete class I haplotype. A summary of the indications, clinical trials, number of patients, and percent of patients with LOH is shown in Table 7. No significant associations were observed in control arms of trials.

TABLE 7
Percent of LOH in clinical trials

| Indication | Trial Name | N | % LOH (any), All arms |
|---|---|---|---|
| NSCLC | IMpower131 | 344 | 24% |
| SCLC | IMpower133 | 190 | 26% |
| NSCLC | IMpower150 | 544 | 16% |
| NSCLC | POPLAR | 148 | 41% |
| mRCC | IMmotion150 | 205 | 62% |

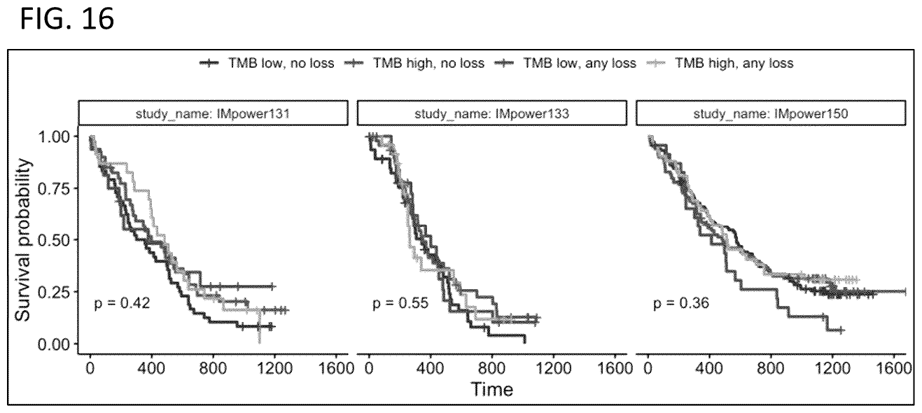

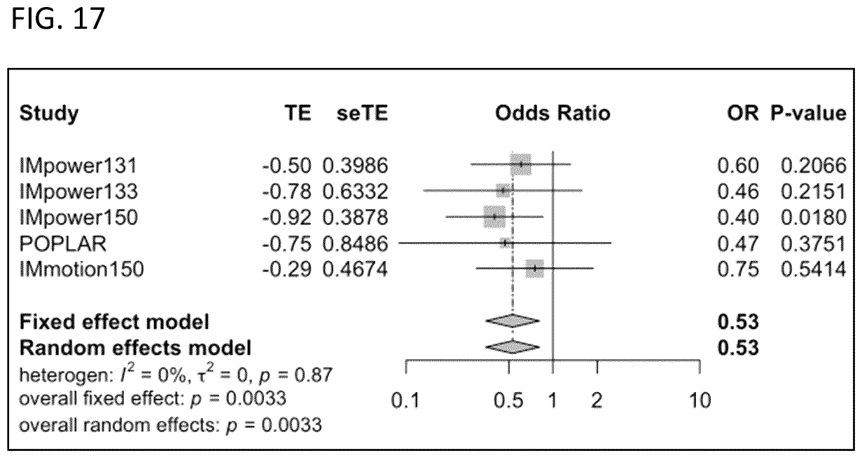

Additionally, tumor mutational burden (TMB) did not modify the impact of class I LOH on outcome (FIG. 16). No differential impact of LOH in the low versus high TMB setting was observed in lung cancer. LOH was not more frequent at intermediate TMB. However, class I LOH was associated with lower CD8A expression (FIG. 17). Without wishing to be bound by theory, these data could indicate that these observations reflect a selective process resulting in decreased CD8+ T cell infiltration.

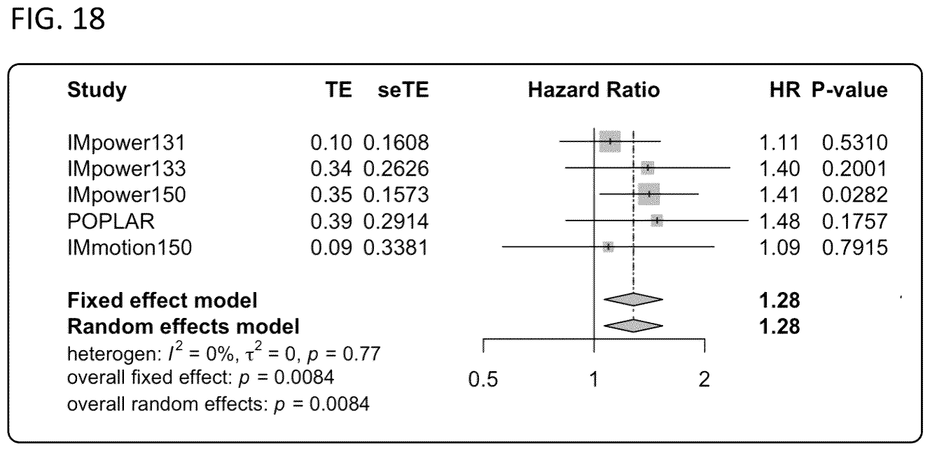

Unexpectedly, HLA class II loss was associated with outcome. In particular, LOH calls for HLA class II genes showed association with shorter OS in a meta analysis (FIG. 18). Without wishing to be bound by theory, this may be consistent with findings suggesting that optimal anti-tumor responses require tumor cells expressing both HLA class I and class II neoantigens.

### OTHER EMBODIMENTS

Although the foregoing invention has been described in some detail by way of illustration and example for purposes of clarity of understanding, the descriptions and examples should not be construed as limiting the scope of the invention.

What is claimed is:

1. A method of treating non-small cell lung cancer (NSCLC) in a patient in need thereof whose genome has been determined to comprise at least one copy of HLA-C1, the method comprising administering to the patient an effective amount of a treatment regimen comprising a PD-1 axis binding antagonist.

2. The method of claim 1, wherein the patient's genome further comprises at least one copy of KIR2DL3.

3. A method of treating NSCLC in a patient in need thereof whose genome has been determined to comprise at least one copy of HLA-C1 and at least one copy of KIR2DL3, the method comprising administering to the patient an effective amount of a treatment regimen comprising a PD-1 axis binding antagonist.

4. A method of treating NSCLC in a patient in need thereof whose genome has been determined to comprise at least one copy of HLA-Bw4, the method comprising administering to the patient an effective amount of a treatment regimen comprising a PD-1 axis binding antagonist.

5. The method of claim 4, wherein the patient's genome further comprises at least one copy of KIR3DL1.

6. A method of treating NSCLC in a patient in need thereof whose genome has been determined to comprise at least one copy of HLA-Bw4 and at least one copy of KIR3DL1, the method comprising administering to the patient an effective amount of a treatment regimen comprising a PD-1 axis binding antagonist.

7. A method of treating NSCLC in a patient in need thereof, the method comprising:
(a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist; and
(b) administering an effective amount of a treatment regimen comprising a PD-1 axis binding antagonist to the patient based on the presence of at least one copy of HLA-C1 in the patient's genome.

8. The method of claim 7, wherein step (a) further comprises determining whether the patient's genome comprises at least one copy of KIR2DL3.

9. A method of treating NSCLC in a patient in need thereof, the method comprising:
(a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist; and
(b) administering an effective amount of a treatment regimen comprising a PD-1 axis binding antagonist to the patient based on the presence of at least one copy of HLA-C1 and at least one copy of KIR2DL3 in the patient's genome.

10. A method of treating NSCLC in a patient in need thereof, the method comprising:
(a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist; and
(b) administering an effective amount of a treatment regimen comprising a PD-1 axis binding antagonist to the patient based on the presence of at least one copy of HLA-Bw4 in the patient's genome.

11. The method of claim 10, wherein step (a) further comprises determining whether the patient's genome comprises at least one copy of KIR3DL1.

12. A method of treating NSCLC in a patient in need thereof, the method comprising:
(a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist; and
(b) administering an effective amount of a treatment regimen comprising a PD-1 axis binding antagonist to the patient based on the presence of at least one copy of HLA-Bw4 and at least one copy of KIR3DL1 in the patient's genome.

13. A method of identifying a patient having NSCLC who may benefit from a treatment regimen comprising a PD-1 axis binding antagonist, the method comprising determining whether the patient's genome comprises at least one copy of HLA-C1, wherein the presence of at least one copy of HLA-C1 in the patient's genome identifies the patient as one who may benefit from treatment with a treatment regimen comprising a PD-1 axis binding antagonist.

14. A method of identifying a patient having NSCLC who may benefit from a treatment regimen comprising a PD-1 axis binding antagonist, the method comprising:
(a) performing germline whole genome sequencing (WGS) or whole exome sequencing (WES) by fragmenting a DNA sample obtained from the patient to produce fragmented DNA, adding adapters to the fragmented DNA to produce one or more libraries, and sequencing the one or more libraries; and
(b) identifying the patient as one who may benefit from a treatment regimen comprising a PD-1 axis binding antagonist by determining whether the patient's genome comprises at least one copy of HLA-C1, wherein the presence of at least one copy of HLA-C1 in the patient's genome identifies the patient as one who may benefit from treatment with a treatment regimen comprising a PD-1 axis binding antagonist.

15. The method of claim 13 or 14, further comprising determining whether the patient's genome comprises at least one copy of KIR2DL3.

16. A method of identifying a patient having NSCLC who may benefit from a treatment regimen comprising a PD-1 axis binding antagonist, the method comprising determining whether the patient's genome comprises at least one copy of HLA-C1 and at least one copy of KIR2DL3, wherein the presence of at least one copy of HLA-C1 and at least one copy of KIR2DL3 in the patient's genome identifies the patient as one who may benefit from treatment with a treatment regimen comprising a PD-1 axis binding antagonist.

17. A method of identifying a patient having NSCLC who may benefit from a treatment regimen comprising a PD-1 axis binding antagonist, the method comprising:
(a) performing germline WGS or WES by fragmenting a DNA sample obtained from the patient to produce fragmented DNA, adding adapters to the fragmented DNA to produce one or more libraries, and sequencing the one or more libraries; and
(b) identifying the patient as one who may benefit from a treatment regimen comprising a PD-1 axis binding antagonist by determining whether the patient's genome comprises at least one copy of HLA-C1 and at least one copy of KIR2DL3, wherein the presence of at least one copy of HLA-C1 and at least one copy of KIR2DL3 in the patient's genome identifies the patient as one who may benefit from treatment with a treatment regimen comprising a PD-1 axis binding antagonist.

18. A method of identifying a patient having NSCLC who may benefit from a treatment regimen comprising a PD-1 axis binding antagonist, the method comprising determining whether the patient's genome comprises at least one copy of HLA-Bw4, wherein the presence of at least one copy of HLA-Bw4 in the patient's genome identifies the patient as one who may benefit from treatment with a treatment regimen comprising a PD-1 axis binding antagonist.

19. A method of identifying a patient having NSCLC who may benefit from a treatment regimen comprising a PD-1 axis binding antagonist, the method comprising:
(a) performing germline WGS or WES by fragmenting a DNA sample obtained from the patient to produce fragmented DNA, adding adapters to the fragmented DNA to produce one or more libraries, and sequencing the one or more libraries; and
(b) identifying the patient as one who may benefit from a treatment regimen comprising a PD-1 axis binding antagonist by determining whether the patient's genome comprises at least one copy of HLA-Bw4, wherein the presence of at least one copy of HLA-Bw4 in the patient's genome identifies the patient as one who may benefit from treatment with a treatment regimen comprising a PD-1 axis binding antagonist.

20. The method of claim 18 or 19, further comprising determining whether the patient's genome comprises at least one copy of KIR3DL1.

21. A method of identifying a patient having NSCLC who may benefit from a treatment regimen comprising a PD-1 axis binding antagonist, the method comprising determining whether the patient's genome comprises at least one copy of HLA-Bw4 and at least one copy of KIR3DL1, wherein the presence of at least one copy of HLA-Bw4 and at least one copy of KIR3DL1 in the patient's genome identifies the patient as one who may benefit from treatment with a treatment regimen comprising a PD-1 axis binding antagonist.

22. A method of identifying a patient having NSCLC who may benefit from a treatment regimen comprising a PD-1 axis binding antagonist, the method comprising:
(a) performing germline WGS or WES by fragmenting a DNA sample obtained from the patient to produce fragmented DNA, adding adapters to the fragmented DNA to produce one or more libraries, and sequencing the one or more libraries; and
(b) identifying the patient as one who may benefit from a treatment regimen comprising a PD-1 axis binding antagonist by determining whether the patient's genome comprises at least one copy of HLA-Bw4 and at least one copy of KIR3DL1, wherein the presence of at least one copy of HLA-Bw4 and at least one copy of KIR3DL1 in the patient's genome identifies the patient as one who may benefit from treatment with a treatment regimen comprising a PD-1 axis binding antagonist.

23. A method for selecting a therapy for a patient having NSCLC, the method comprising:
(a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist; and
(b) selecting a treatment regimen comprising a PD-1 axis binding antagonist based on the presence of at least one copy of HLA-C1 in the patient's genome.

24. The method of claim 23, further comprising determining whether the patient's genome comprises at least one copy of KIR2DL3.

25. A method for selecting a therapy for a patient having NSCLC, the method comprising:
(a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist; and
(b) selecting a treatment regimen comprising a PD-1 axis binding antagonist based on the presence of at least one copy of HLA-C1 and at least one copy of KIR2DL3 in the patient's genome.

26. A method for selecting a therapy for a patient having NSCLC, the method comprising:
(a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist; and
(b) selecting a treatment regimen comprising a PD-1 axis binding antagonist based on the presence of at least one copy of HLA-Bw4 in the patient's genome.

27. The method of claim 26, further comprising determining whether the patient's genome comprises at least one copy of KIR3DL1.

28. A method for selecting a therapy for a patient having NSCLC, the method comprising:
(a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist; and
(b) selecting a treatment regimen comprising a PD-1 axis binding antagonist based on the presence of at least one copy of HLA-Bw4 and at least one copy of KIR3DL1 in the patient's genome.

29. The method of any one of claims 13-28, further comprising administering an effective amount of a treatment regimen comprising a PD-1 axis binding antagonist to the patient.

30. The method of any one of claims 1-29, wherein the presence of HLA-C1, HLA-Bw4, KIR2DL3, and/or KIR3DL1 in the patient's genome is determined using next-generation sequencing, Sanger sequencing, a polymerase chain reaction (PCR)-based assay, or a single nucleotide polymorphism (SNP) array.

31. The method of claim 30, wherein the next-generation sequencing comprises germline whole-genome sequencing or germline whole-exome sequencing.

32. The method of claim 30, wherein the PCR-based assay comprises quantitative PCR (qPCR), typing using sequence-specific primers (SSP), or typing using sequence specific oligonucleotide probes (SSO).

33. A method of treating NSCLC in a patient in need thereof who has been determined to have an increased level of natural killer (NK) cell infiltration in a tumor sample obtained from the patient relative to a reference level of NK cell infiltration, the method comprising administering to the patient an effective amount of a treatment regimen comprising a PD-1 axis binding antagonist.

34. A method of treating NSCLC in a patient in need thereof, the method comprising:
(a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist; and
(b) administering an effective amount of a treatment regimen comprising a PD-1 axis binding antagonist to the patient based on the increased level of NK cell infiltration in the tumor sample obtained from the patient relative to the reference level of NK cell infiltration.

35. A method of identifying a patient having NSCLC who may benefit from a treatment regimen comprising a PD-1 axis binding antagonist, the method comprising determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist.

36. A method of identifying a patient having NSCLC who may benefit from a treatment regimen comprising a PD-1 axis binding antagonist, the method comprising:
(a) contacting a tumor sample obtained from the patient with one or more antibodies or nucleotide probes that bind to one or more NK cell markers to determine the level of NK cell infiltration in the tumor sample; and
(b) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist.

37. A method for selecting a therapy for a patient having NSCLC, the method comprising:
(a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist; and
(b) selecting a treatment regimen comprising a PD-1 axis binding antagonist based on the increased level of NK cell infiltration in the tumor sample obtained from the patient relative to the reference level of NK cell infiltration.

38. The method of any one of claims 35-37, further comprising administering an effective amount of a treatment regimen comprising a PD-1 axis binding antagonist to the patient.

39. The method of any one of claims 33-38, wherein the level of NK cell infiltration is determined by determining an expression level of an NK cell gene signature or by counting a number of NK cells in the tumor sample.

40. The method of claim 39, wherein the NK cell gene signature comprises one or more of the following genes: CD160, CD244, CTSW, FASLG, GZMA, GZMB, GZMH, IL18RAP, IL2RB, KIR2DL4, KLRB1, KLRC3, KLRD1, KRLF1, KLRK1, NCR1, NKG7, PRF1, XCL1, and XCL2.

41. The method of claim 40, wherein the NK cell gene signature comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of the genes.

42. The method of any one of claims 33-41, wherein the reference level of NK cell infiltration is a median level.

43. The method of claim 42, wherein the median level is a median level in a population of NSCLC patients.

44. The method of any one of claims 7-32 and 34-43, wherein the benefit is in terms of improved overall survival (OS) or improved progression-free survival (PFS).

45. The method of claim 44, wherein the benefit is in terms of improved OS.

46. The method of claim 44, wherein the benefit is in terms of improved PFS.

47. The method of any one of claims 44-46, wherein improvement is relative to treatment with a treatment regimen that does not comprise the PD-1 axis binding antagonist.

48. The method of any one of claims 1-47, wherein the NSCLC is non-squamous NSCLC or squamous NSCLC.

49. The method of claim 48, wherein the NSCLC is non-squamous NSCLC.

50. The method of claim 49, wherein the non-squamous NSCLC is metastatic non-squamous NSCLC.

51. The method of claim 48, wherein the NSCLC is squamous NSCLC.

52. The method of claim 51, wherein the squamous NSCLC is metastatic squamous NSCLC.

53. The method of any one of claims 1-52, wherein the patient is chemotherapy-naïve.

54. The method of any one of claims 1-53, wherein the treatment regimen is a first-line treatment regimen.

55. The method of any one of claims 1-54, wherein the PD-1 axis binding antagonist is selected from a PD-L1 binding antagonist, a PD-1 binding antagonist, and a PD-L2 binding antagonist.

56. The method of claim 55, wherein the PD-1 axis binding antagonist is a PD-L1 binding antagonist.

57. The method of claim 56, wherein the PD-L1 binding antagonist is an anti-PD-L1 antibody.

58. The method of claim 57, wherein the anti-PD-L1 antibody comprises (a) a hypervariable region (HVR)-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

59. The method of claim 57 or 58, wherein the anti-PD-L1 antibody comprises
(a) a VH comprising the amino acid sequence:
(SEQ ID NO: 9)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,
and
(b) the VL comprising the amino acid sequence:
(SEQ ID NO: 10)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

60. The method of claim 57, wherein the anti-PD-L1 antibody is atezolizumab, durvalumab, avelumab, or MDX-1105.

61. The method of any one of claims 57-60, wherein the anti-PD-L1 antibody is atezolizumab.

62. The method of any one of claims 57-61, wherein the anti-PD-L1 antibody is administered intravenously or subcutaneously.

63. The method of claim 61, wherein the atezolizumab is administered intravenously every two weeks at a dose of 840 mg.

64. The method of claim 61, wherein the atezolizumab is administered intravenously every three weeks at a dose of 1200 mg.

65. The method of claim 61, wherein the atezolizumab is administered intravenously every four weeks at a dose of 1680 mg.

66. The method of claim 55, wherein the PD-1 axis binding antagonist is a PD-1 binding antagonist.

67. The method of claim 66, wherein the PD-1 binding antagonist is an anti-PD-1 antibody.

68. The method of claim 67, wherein the anti-PD-1 antibody is nivolumab, pembrolizumab, MEDI-0680, spartalizumab, cemiplimab, camrelizumab, sintilimab, tislelizumab, toripalimab, or dostarlimab.

69. The method of any one of claims 1-68, wherein the treatment regimen further comprises a taxane.

70. The method of claim 69, wherein the taxane is nab-paclitaxel or paclitaxel.

71. The method of claim 70, wherein the taxane is nab-paclitaxel.

72. The method of claim 70, wherein the taxane is paclitaxel.

73. The method of any one of claims 1-72, wherein the treatment regimen further comprises a platinum-based chemotherapeutic agent.

74. The method of claim 73, wherein the platinum-based chemotherapeutic agent is carboplatin.

75. The method of any one of claims 1-74, wherein the treatment regimen further comprises an anti-angiogenic agent.

76. The method of claim 75, wherein the anti-angiogenic agent is an anti-VEGF antibody.

77. The method of claim 76, wherein the anti-VEGF antibody is bevacizumab.

78. The method of any one of claims 1-50 and 53-77, wherein the NSCLC is metastatic non-squamous NSCLC, and the treatment regimen comprises atezolizumab, nab-paclitaxel, and carboplatin.

79. The method of claim 78, wherein atezolizumab is administered as an intravenous (IV) infusion at a dose of 1200 mg on Day 1 of each 21-day cycle; nab-paclitaxel is administered as an IV infusion at a dose of 100 mg/m² on Days 1, 8, and 15 of each 21-day cycle; and carboplatin is administered at an area under the concentration curve (AUC) of 6 mg/mL/min on Day 1 of each 21-day cycle.

80. The method of any one of claims 1-50 and 53-77, wherein the NSCLC is metastatic non-squamous NSCLC, and the treatment regimen comprises atezolizumab, paclitaxel, and carboplatin.

81. The method of claim 80, wherein atezolizumab is administered as an IV infusion at a dose of 1200 mg on Day 1 of each 21-day cycle; paclitaxel is administered as an IV infusion at a dose of 200 mg/m² on Day 1 each 21-day cycle; and carboplatin is administered at an AUC of 6 mg/mL/min on Day 1 of each 21-day cycle.

82. The method of any one of claims 1-50 and 53-77, wherein the NSCLC is metastatic non-squamous NSCLC, and the treatment regimen comprises atezolizumab, bevacizumab, paclitaxel, and carboplatin.

83. The method of claim 82, wherein atezolizumab is administered as an IV infusion at a dose of 1200 mg on Day 1 of each 21-day cycle; bevacizumab is administered as an IV infusion at a dose of 15 mg/kg on Day 1 of each 21-day cycle; paclitaxel is administered as an IV infusion at a dose of 200 mg/m² on Day 1 each 21-day cycle; and carboplatin is administered at an AUC of 6 mg/mL/min on Day 1 of each 21-day cycle.

84. The method of any one of claims 1-48 and 51-77, wherein the NSCLC is metastatic squamous NSCLC, and the treatment regimen comprises atezolizumab, nab-paclitaxel, and carboplatin.

85. The method of claim 84, wherein atezolizumab is administered as an IV infusion at a dose of 1200 mg on Day 1 of each 21-day cycle; nab-paclitaxel is administered as an IV infusion at a dose of 100 mg/m² on Days 1, 8, and 15 of each 21-day cycle; and carboplatin is administered at an area under the concentration curve (AUC) of 6 mg/mL/min on Day 1 of each 21-day cycle.

86. The method of any one of claims 1-48 and 51-77, wherein the NSCLC is metastatic squamous NSCLC, and the treatment regimen comprises atezolizumab, paclitaxel, and carboplatin.

87. The method of claim 86, wherein atezolizumab is administered as an IV infusion at a dose of 1200 mg on Day 1 of each 21-day cycle; paclitaxel is administered as an IV infusion at a dose of 175 mg/m² or 200 mg/m² on Days 1, 8, and 15 of each 21-day cycle; and carboplatin is administered at an AUC of 6 mg/mL/min on Day 1 of each 21-day cycle.

88. The method of any one of claims 1-87, further comprising administering an additional therapeutic agent to the patient.

89. The method of claim 88, wherein the additional therapeutic agent is selected from the group consisting of an immunotherapy agent, a cytotoxic agent, a growth inhibitory agent, a radiation therapy agent, an anti-angiogenic agent, and combinations thereof.

90. A PD-1 axis binding antagonist for use in treating NSCLC in a patient in need thereof whose genome has been determined to comprise at least one copy of HLA-C1.

91. The PD-1 axis binding antagonist for use of claim 90, wherein the patient's genome further comprises at least one copy of KIR2DL3.

92. A PD-1 axis binding antagonist for use in treating NSCLC in a patient in need thereof whose genome has been determined to comprise at least one copy of HLA-C1 and at least one copy of KIR2DL3.

93. A PD-1 axis binding antagonist for use in treating NSCLC in a patient in need thereof whose genome has been determined to comprise at least one copy of HLA-Bw4.

94. The PD-1 axis binding antagonist for use of claim 93, wherein the patient's genome further comprises at least one copy of KIR3DL1.

95. A PD-1 axis binding antagonist for use in treating NSCLC in a patient in need thereof whose genome has been determined to comprise at least one copy of HLA-Bw4 and at least one copy of KIR3DL1.

96. A PD-1 axis binding antagonist for use in a method of treating NSCLC in a patient in need thereof, the method comprising:
(a) determining whether the patient's genome comprises at least one copy of HLA-C1, wherein the presence of at least one copy of HLA-C1 in the patient's genome indicates that the patient is likely to benefit from a treatment regimen comprising a PD-1 axis binding antagonist; and
(b) administering an effective amount of a treatment regimen comprising a PD-1 axis binding antagonist to the patient based on the presence of at least one copy of HLA-C1 in the patient's genome.

97. The PD-1 axis binding antagonist for use of claim 96, wherein step (a) further comprises determining whether the patient's genome comprises at least one copy of KIR2DL3.

98. A PD-1 axis binding antagonist for use in treating NSCLC in a patient in need thereof, the method comprising:
(a) determining whether the patient's genome comprises at least one copy of HLA-C1 and at least one copy of KIR2DL3, wherein the presence of at least one copy of HLA-C1 and at least one copy of KIR2DL3 in the patient's genome indicates that the patient is likely to benefit from a treatment regimen comprising a PD-1 axis binding antagonist; and
(b) administering an effective amount of a treatment regimen comprising a PD-1 axis binding antagonist to the patient based on the presence of at least one copy of HLA-C1 and at least one copy of KIR2DL3 in the patient's genome.

99. A PD-1 axis binding antagonist for use in treating NSCLC in a patient in need thereof, the method comprising:
(a) determining whether the patient's genome comprises at least one copy of HLA-Bw4, wherein the presence of at least one copy of HLA-Bw4 in the patient's genome indicates that the patient is likely to benefit from a treatment regimen comprising a PD-1 axis binding antagonist; and
(b) administering an effective amount of a treatment regimen comprising a PD-1 axis binding antagonist to the patient based on the presence of at least one copy of HLA-Bw4 in the patient's genome.

100. The PD-1 axis binding antagonist for use of claim 99, wherein step (a) further comprises determining whether the patient's genome comprises at least one copy of KIR3DL1.

101. A PD-1 axis binding antagonist for use in treating NSCLC in a patient in need thereof, the method comprising:
(a) determining whether the patient's genome comprises at least one copy of HLA-Bw4 and at least one copy of KIR3DL1, wherein the presence of at least one copy of HLA-Bw4 and at least one copy of KIR3DL1 in the patient's genome indicates that the patient is likely to benefit from a treatment regimen comprising a PD-1 axis binding antagonist; and
(b) administering an effective amount of a treatment regimen comprising a PD-1 axis binding antagonist to the patient based on the presence of at least one copy of HLA-Bw4 and at least one copy of KIR3DL1 in the patient's genome.

102. A PD-1 axis binding antagonist for use in treating NSCLC in a patient in need thereof who has been determined to have an increased level of NK cell infiltration in a tumor sample obtained from the patient relative to a reference level of NK cell infiltration.

103. A PD-1 axis binding antagonist for use in a method of treating NSCLC in a patient in need thereof, the method comprising:
(a) determining whether a tumor sample obtained from the patient has an increased level of NK cell infiltration relative to a reference level of NK cell infiltration, wherein an increased level of NK cell infiltration in the tumor sample obtained from the patient relative to the reference level of NK cell infiltration indicates that the patient is likely to benefit from a treatment regimen comprising a PD-1 axis binding antagonist; and
(b) administering an effective amount of a treatment regimen comprising a PD-1 axis binding antagonist to the patient based on the increased level of NK cell infiltration in the tumor sample obtained from the patient relative to the reference level of NK cell infiltration.

104. A NK cell-directed therapy agent for use in treating NSCLC in a patient in need thereof whose genome has been determined to lack KIR2DL3 or KIR3DL1.

105. A NK cell-directed therapy agent for use in a method of treating NSCLC in a patient in need thereof, the method comprising:
(a) determining whether the patient's genome lacks KIR2DL3 or KIR3DL1, wherein the absence of KIR2DL3 or KIR3DL1 in the patient's genome indicates that the patient is likely to benefit from a treatment regimen comprising an NK cell-directed therapy agent; and
(b) administering an effective amount of a treatment regimen comprising an NK cell-directed therapy agent to the patient based on the absence of KIR2DL3 or KIR3DL1 in the patient's genome.

106. An article of manufacture comprising a PD-1 axis binding antagonist and instructions to administer the PD-1 axis binding antagonist for treatment of NSCLC in a patient in need thereof whose genome has been determined to comprise at least one copy of HLA-C1.

107. The article of manufacture of claim 106, wherein the patient's genome further comprises at least one copy of KIR2DL3.

108. An article of manufacture comprising a PD-1 axis binding antagonist and instructions to administer the PD-1 axis binding antagonist for treatment of NSCLC in a patient in need thereof whose genome has been determined to comprise at least one copy of HLA-C1 and at least one copy of KIR2DL3.

109. An article of manufacture comprising a PD-1 axis binding antagonist and instructions to administer the PD-1 axis binding antagonist for treatment of NSCLC in a patient in need thereof whose genome has been determined to comprise at least one copy of HLA-Bw4.

110. The article of manufacture of claim 109, wherein the patient's genome further comprises at least one copy of KIR3DL1.

111. An article of manufacture comprising a PD-1 axis binding antagonist and instructions to administer the PD-1 axis binding antagonist for treatment of treating NSCLC in a patient in need thereof whose genome has been determined to comprise at least one copy of HLA-Bw4 and at least one copy of KIR3DL1.

112. An article of manufacture comprising a PD-1 axis binding antagonist and instructions to administer the PD-1 axis binding antagonist for treatment of NSCLC in a patient in need thereof who has been determined to have an increased level of natural killer (NK) cell infiltration in a tumor sample obtained from the patient relative to a reference level of NK cell infiltration.

113. An article of manufacture comprising an NK cell-directed therapy agent and instructions to administer the NK cell-directed therapy agent for treatment of NSCLC in a patient in need thereof whose genome has been determined to lack KIR2DL3 or KIR3DL1.


SEQUENCE LISTING (10 sequences)

SEQ ID NO: 1 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
GFTFSDSWIH

SEQ ID NO: 4 - protein, 18 aa, synthetic construct
AWISPYGGSTYYADSVKG

SEQ ID NO: 5 - protein, 9 aa, synthetic construct
RHWPGGFDY

SEQ ID NO: 6 - protein, 11 aa, synthetic construct
RASQDVSTAVA

SEQ ID NO: 7 - protein, 7 aa, synthetic construct
SASFLYS

SEQ ID NO: 8 - protein, 9 aa, synthetic construct
QQYLYHPAT

SEQ ID NO: 9 - protein, 118 aa, synthetic construct [sequence omitted]

SEQ ID NO: 10 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR